(19) United States
(12) Patent Application Publication
(10) Pub. No.: US 2026/0345354 A1
(43) Pub. Date: Nov. 5, 2026

(54) BI-DIRECTIONAL INTERFACE WITH ELECTRODES IN DIFFERENT TISSUES FOR COMPOSITE REGENERATIVE PERIPHERAL NERVE INTERFACE (C-RPNI)

(71) Applicant: The Regents of The University of Michigan, Ann Arbor, MI (US)

(72) Inventors: Paul S. CEDERNA, Milan, MI (US); Stephen KEMP, Ann Arbor, MI (US); Theodore A. KUNG, Ann Arbor, MI (US); Alex VASKOV, Ann Arbor, MI (US); Widya ADIDHARMA, Ann Arbor, MI (US); Carrie KUBIAK, Palo Alto, CA (US); Shelby SVIENTEK, Ann Arbor, MI (US); Cynthia Anne CHESTEK, Ann Arbor, MI (US)

(73) Assignee: The Regents of The University of Michigan, Ann Arbor, MI (US)

(21) Appl. No.: 19/485,024

(22) PCT Filed: May 17, 2024
(86) PCT No.: PCT/US2024/029881

§ 371 Date: Nov. 17, 2025

Related U.S. Application Data
(60) Provisional application No. 63/467,499, filed on May 18, 2023.

(51) Int. Cl. A61F 2/72; A61F 2/08; A61F 2/10; A61F 2/50; A61F 2/70; A61N 1/05; A61N 1/36

(52) U.S. Cl. CPC A61F 2/72; A61F 2/105; A61N 1/0551; A61N 1/36135; A61F 2002/0894; A61F 2002/5061; A61F 2002/704; A61F 2250/0002; A61N 1/36103

(57) ABSTRACT

Systems and methods include a composite tissue graft having a muscle graft portion and a dermal graft portion surgically attached to surround a nerve of a subject. A sensing electrode is attached to the muscle graft portion and a stimulating electrode is attached to the dermal graft portion. An implant device includes sensing processing circuitry is configured to generate processed motor signal data corresponding to electrical signals received from the at least one sensing electrode, and to transmit the processed motor signal data to a prosthetic controller that controls a prosthetic device based on the processed motor signal data. The stimulating processing circuitry is configured to receive sensory feedback data from the prosthetic controller, generate processed sensory signal data based on the sensory feedback data, and transmit electrical signals to the stimulating electrode corresponding to the processed sensory signal data.

### CROSS-REFERENCE TO RELATED APPLICATIONS

This application claims the benefit of U.S. Provisional Application No. 63/467,499, filed on May 18, 2023. The entire disclosure of the above application is incorporated herein by reference.

### GOVERNMENT LICENSE RIGHTS

This invention was made with government support under W81XWH-21-1-0429 awarded by the U.S. Army Medical Research and Development Command, and NS104584 awarded by the National Institutes of Health. The government has certain rights in the invention.

### FIELD

The present disclosure relates to regenerative peripheral nerve interface (RPNI) devices.

### BACKGROUND

This section provides background information related to the present disclosure which is not necessarily prior art.

There is a need to receive and record signals from nerves (for example, human nerves) for subsequent processing and use in, for example, controlling prosthetic limbs. A free tissue graft can be attached to a portion of a nerve, such as a nerve fascicle, and electrical signals from the nerve can be amplified by the free tissue graft. Systems and methods are needed, however, to effectively and efficiently process the amplified signals from the nerve and to effectively and efficiently control a prosthetic based on the processed signals. In addition, systems and methods are needed to effectively and efficiently transmit sensory feedback signals from a prosthetic to the nerve through the free tissue graft.

### SUMMARY

This section provides a general summary of the disclosure and is not a comprehensive disclosure of its full scope or all of its features.

A system includes an implant device having sensing processing circuitry configured to receive electrical signals from a composite tissue graft surgically attached to surround a nerve of a subject and having stimulating processing circuitry configured to transmit electrical signals to the composite tissue graft, the composite tissue graft including a muscle graft portion and a dermal graft portion, the composite tissue graft being surgically attached to the subject such that the composite tissue graft is entirely surrounded by and in direct contact with non-grafted tissue of the subject, the muscle graft potion and the dermal graft portion each being an autograft of tissue harvested from the subject, devascularized, and deinnervated prior to being surgically attached to the subject, the nerve having reinnervated the muscle graft portion and the dermal graft portion of the composite tissue graft subsequent to the composite tissue graft being surgically attached to the nerve. At least one sensing electrode is attached to, and in electrical communication with, the muscle graft portion of the composite tissue graft. At least one stimulating electrode is attached to, and in electrical communication with, the dermal graft portion of the composite tissue graft. The sensing processing circuitry is configured to receive electrical signals from the at least one sensing electrode, process the received electrical signals from the at least one sensing electrode, generate processed motor signal data corresponding to electrical signals received from the at least one sensing electrode, and to transmit the processed motor signal data to a prosthetic controller, the prosthetic controller being configured to control a prosthetic device based on the processed motor signal data transmitted from the sensing processing circuitry of the implant device. The stimulating processing circuitry is configured to receive sensory feedback data from the prosthetic controller, generate processed sensory signal data based on the sensory feedback data, and transmit electrical signals to the at least one stimulating electrode corresponding to the processed sensory signal data.

A method includes receiving, with sensing processing circuitry of an implant device, electrical signals from a composite tissue graft surgically attached to surround a nerve of a subject. The method also includes transmitting, with stimulating processing circuitry of the implant device, electrical signals to the composite tissue graft, the composite tissue graft including a muscle graft portion and a dermal graft portion, the composite tissue graft being surgically attached to the subject such that the composite tissue graft is entirely surrounded by and in direct contact with non-grafted tissue of the subject, the muscle graft potion and the dermal graft portion each being an autograft of tissue harvested from the subject, devascularized, and deinnervated prior to being surgically attached to the subject, the nerve having reinnervated the muscle graft portion and the dermal graft portion of the composite tissue graft subsequent to the composite tissue graft being surgically attached to the nerve. At least one sensing electrode is attached to, and in electrical communication with, the muscle graft portion of the composite tissue graft. At least one stimulating electrode is attached to, and in electrical communication with, the dermal graft portion of the composite tissue graft.

The sensing processing circuitry is configured to receive electrical signals from the at least one sensing electrode, process the received electrical signals from the at least one sensing electrode, generate processed motor signal data corresponding to electrical signals received from the at least one sensing electrode, and to transmit the processed motor signal data to a prosthetic controller, the prosthetic controller being configured to control a prosthetic device based on the processed motor signal data transmitted from the sensing processing circuitry of the implant device. The stimulating processing circuitry is configured to receive sensory feedback data from the prosthetic controller, generate processed sensory signal data based on the sensory feedback data, and transmit electrical signals to the at least one stimulating electrode corresponding to the processed sensory signal data.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 illustrates a regenerative peripheral nerve interface in accordance with certain aspects of the present disclosure.

FIG. 2 illustrates three free muscle grafts after being harvested from a subject and a photograph showing a surgical procedure for surgically attaching the free muscle grafts to nerve fascicles of a subject in accordance with certain aspects of the present disclosure.

FIG. 3 illustrates another regenerative peripheral nerve interface and a prosthetic device in accordance with certain aspects of the present disclosure.

FIG. 4 illustrates another regenerative peripheral nerve interface in accordance with certain aspects of the present disclosure.

FIG. 5 is a block diagram of an implant device in accordance with certain aspects of the present disclosure.

FIG. 6 is a photograph of a hardware device chip in accordance with certain aspects of the present disclosure.

FIG. 7 is a block diagram of processing circuitry with memory and communication circuitry in accordance with certain aspects of the present disclosure.

FIG. 8 is a flowchart depicting an example control algorithm for recording amplified nerve signal data in accordance with certain aspects of the present disclosure.

FIG. 9 is a flowchart depicting an example control algorithm for control of a prosthetic limb in accordance with certain aspects of the present disclosure.

FIG. 10 is a flowchart depicting an example control algorithm for decoding signal for control of a prosthetic limb in accordance with certain aspects of the present disclosure.

FIG. 11 is a graph showing nerve signal data corresponding to onset of a flexion movement.

FIG. 12 is a flowchart depicting an example control algorithm for monitoring nerves for pathological pain signals in accordance with certain aspects of the present disclosure.

FIG. 13 is a flowchart depicting an example control algorithm for monitoring pathological bladder contraction signals in accordance with certain aspects of the present disclosure.

FIG. 14 a flowchart depicting an example control algorithm for stimulating nerves based on sensed pressure signal from a prosthetic device in accordance with certain aspects of the present disclosure.

FIG. 15 is a graph showing nerve signal data corresponding to onset of a finger flexion movement.

FIG. 16 is a group of graphs showing nerve signal data from implanted regenerative peripheral nerve interfaces.

FIG. 17 is a group of graphs showing nerve signal data from implanted regenerative peripheral nerve interfaces and from a control muscle.

FIG. 18 is a graph showing nerve signal data from an implanted regenerative peripheral nerve interface.

FIG. 19 is a graph of predicted and actual finger flexion percentages over time.

FIG. 20 illustrates a system for controlling a prosthetic based on amplified nerve signals in accordance with the present disclosure.

FIG. 21 further illustrates the system for controlling a prosthetic based on amplified nerve signals in accordance with the present disclosure.

FIG. 22 illustrates electrical leads of the system for controlling a prosthetic based on amplified nerve signals in accordance with the present disclosure.

FIG. 23 further illustrates the electrical leads of the system for controlling a prosthetic based on amplified nerve signals in accordance with the present disclosure.

FIG. 24A illustrates an implant device including a header to receive eight-contact connectors in accordance with the present disclosure.

FIG. 24B illustrates another embodiment of the electrical leads of the system for controlling a prosthetic based on amplified nerve signals in accordance with the present disclosure.

FIG. 25 illustrates protection circuitry for the implant device in accordance with the present disclosure.

FIG. 26 illustrates a functional block diagram for processing amplified nerve signals in accordance with the present disclosure.

FIG. 27 illustrates a prosthetic controller and prosthetic hand in accordance with the present disclosure.

FIG. 28 illustrates an external programmer charger and inductive charging pad in accordance with the present disclosure.

FIG. 29 illustrates an external programmer device in communication with an implant device and a prosthetic controller in accordance with the present disclosure.

FIG. 30 illustrates another embodiment of a system for controlling a prosthetic based on amplified nerve signals in accordance with the present disclosure.

FIG. 31 illustrates an algorithm for generating prosthetic movement commands and performing stimulation in alternating time periods.

FIG. 32 illustrates another algorithm for generating prosthetic movement commands and performing stimulation in alternating time periods while generating estimates for prosthetic movement commands during the time periods when stimulation is being performed.

FIG. 33 illustrates an algorithm for generating prosthetic movement commands and performing stimulation while performing artifact estimation and subtraction to remove artifacts from sensed signals caused by stimulation.

FIG. 34 illustrates another regenerative peripheral nerve interface in accordance with certain aspects of the present disclosure.

FIG. 35 illustrates histological imaging showing different motor units forming neuromuscular junctions within a single RPNI in accordance with the present disclosure.

FIG. 36 illustrates different regions of RPNIs on a median nerve in response to different individual finger and thumb movements when viewed under ultrasound in accordance with the present disclosure.

FIG. 37 illustrates different regions of RPNIs on an ulnar nerve in response to different individual finger and thumb movements when viewed under ultrasound in accordance with the present disclosure.

FIG. 38A illustrates graphs showing signals for small finger flexion and finger adduction in accordance with the present disclosure.

FIG. 38B illustrates a block diagram for signal processing of RPNI signals to generate movement commands in accordance with the present disclosure.

FIG. 39 illustrates a block diagram showing processing circuitry for signal processing of RPNI signals to generate movement commands in accordance with the present disclosure.

FIG. 40 illustrates multiple sensing electrodes with an RPNI in accordance with the present disclosure.

FIG. 41 illustrates processing circuitry receiving input from multiple input channels of RPNIs in accordance with the present disclosure.

FIG. 42 illustrates multiple networked modules of an implant device receiving input from multiple input channels of RPNIs in accordance with the present disclosure.

FIGS. 43A and 43B are photographs of RPNIs with multiple sensing electrodes in accordance with the present disclosure.

FIG. 44 illustrates a graph showing sensed signals from multiple sensing electrodes of an RPNI in accordance with the present disclosure.

FIG. 45 illustrates a photograph of an RPNI having multiple sensing electrodes in accordance with the present disclosure.

FIG. 46 illustrates a graph showing sensed signals from multiple sensing electrodes of an RPNI in accordance with the present disclosure.

FIG. 47 illustrates a composite regenerative peripheral nerve interface (C-RPNI) in accordance with certain aspects of the present disclosure.

FIG. 48 illustrates a block diagram of an implant device with processing circuitry and multiple C-RPNIs in accordance with the present disclosure.

FIG. 49 illustrates a graph of signals collected by alternating stimulation and recording on the same nerve in accordance with the present disclosure.

FIG. 50 illustrates a graph showing stable sensory detection thresholds for a patient's RPNIs over time in accordance with the present disclosure.

FIG. 51 illustrates hand maps depicting somatotopic accuracy of referred sensation in accordance with the present disclosure.

FIG. 52 illustrates another composite regenerative peripheral nerve interface (C-RPNI) in accordance with certain aspects of the present disclosure.

FIG. 53 illustrates a block diagram of processing circuitry and multiple C-RPNIs in accordance with the present disclosure.

FIG. 54 illustrates another block diagram of processing circuitry and multiple C-RPNIs in accordance with the present disclosure.

FIG. 55 illustrates a block diagram of a high channel count sensing and stimulation implant in accordance with the present disclosure.

FIG. 56 illustrates multiple networked modules of an implant device and multiple C-RPNIs in accordance with the present disclosure.

FIG. 57 illustrates multiple networked modules of an implant device and multiple C-RPNIs in accordance with the present disclosure.

FIG. 58 is a photograph of a photograph of a C-RPNI construct.

FIG. 59 is a photograph of an electrodiagnostic setup for generating and recording by stimulating a dermal graft and recording afferent activity with a nerve cuff in accordance with the present disclosure.

FIG. 60 illustrates a graph with data collected by alternating stimulation and recording on the same nerve in accordance with the present disclosure.

FIG. 61 illustrates a composite cuff regenerative peripheral nerve interface (CC-RPNI) in accordance with certain aspects of the present disclosure.

FIG. 62 is a photograph of a CC-RPNI construct in accordance with the present disclosure.

FIG. 63 is a photograph of a setup for generating and recording signals with a CC-RPNI in accordance with the present disclosure.

FIG. 64 is a graph that illustrates afferent and efferent signaling with a CC-RPNI construct in accordance with the present disclosure.

FIG. 65 illustrates a setup of electrophysiology experiments on a C-RPNI construct in accordance with the present disclosure.

FIG. 66 illustrates graphs showing afferent nerve action potential and electrical stimulus in accordance with the present disclosure.

FIG. 67 illustrates a construct having a single tissue that circumferentially surrounds a nerve physically separated from a different tissue attached to the end of the nerve in accordance with the present disclosure.

FIG. 68 is a photograph of a construct having a single tissue that circumferentially surrounds a nerve physically separated from a different tissue attached to the end of the nerve in accordance with the present disclosure.

FIG. 69 illustrates graphs showing afferent nerve action of a construct having a single tissue that circumferentially surrounds a nerve physically separated from a different tissue attached to the end of the nerve in accordance with the present disclosure.

FIG. 70 illustrates a graph showing afferent and efferent signaling of a construct having a single tissue that circumferentially surrounds a nerve physically separated from a different tissue attached to the end of the nerve in accordance with the present disclosure.

Corresponding reference numerals indicate corresponding parts throughout the several views of the drawings.

It should be noted that the figures set forth herein are intended to exemplify the general characteristics of methods, devices, and materials, among those of the present disclosure, for the purpose of the description of certain embodiments.

These figures may not precisely reflect the characteristics of any given embodiment, and are not necessarily intended to fully define or limit specific embodiments within the scope of this disclosure.

### DETAILED DESCRIPTION

Example embodiments will now be described more fully with reference to the accompanying drawings. A non-limiting discussion of terms and phrases intended to aid understanding of the present disclosure is provided at the end of this Detailed Description.

In various aspects, the present disclosure provides methods for amplifying and receiving signals from a portion of a nerve, such as individual nerve fascicles, at levels greater than that produced by any conventional methods or techniques. Specifically, as described in further detail below, the present disclosure provides methods for amplifying and receiving signals from a portion of a nerve, like individual nerve fascicles, at greater than or equal to about 150 μV pp and, in some instances, to greater than or equal to about 250 or 500 μV pp and up to, for example, about 1,000 μV pp or more. As mentioned above, signals detected by previous neural interface systems typically were less than 100 μV pp when recording from within the nerve and less than 10 μV pp when recording from a cuff around the nerve. In certain aspects, the present disclosure provides implantable nerve interface devices, also referred to interchangeably as regenerative peripheral nerve interface (RPNI) devices, that facilitate amplification of signals from individual nerve fascicles to greater than or equal to about 150 μV pp and, in some instances, to greater than or equal to about 250 or 500 μV pp and up to, for example, about 1,000 μV pp or more.

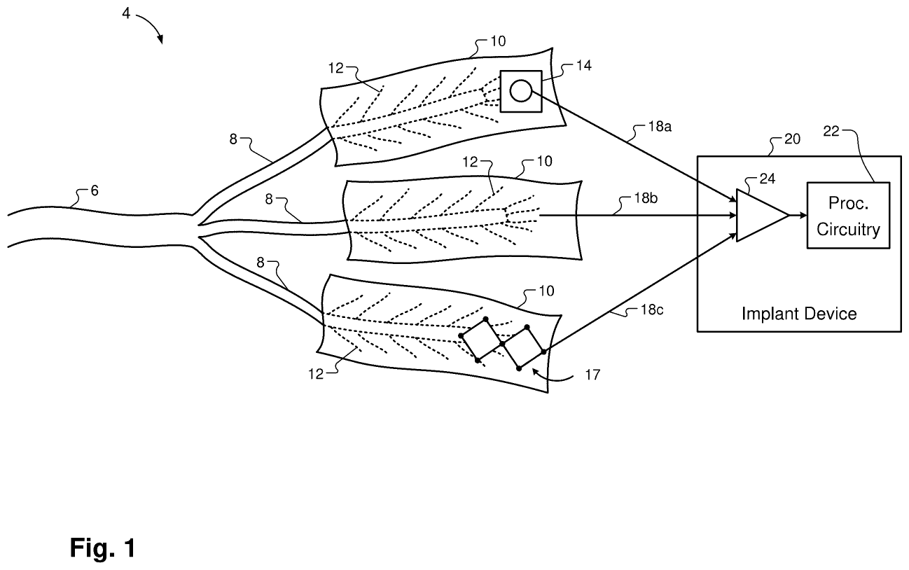

With reference to FIG. 1, a neural interface system 4 in a subject or patient is shown. The subject may be an animal with a complex nerve system, such as a mammal, like a human, primate, or companion animal. A portion of a nerve 6, such as a nerve end, of the subject may be damaged or severed, for example, a fully or partially lesioned nerve end caused by injury, disease, or surgery. In certain aspects, the method may include surgically dividing, sectioning, cutting, and/or transecting a portion of a nerve 6 into one or more individual branches or fascicles 8. It should be noted that in certain variations, the method may include isolating a portion of a nerve 6 of interest to create the one or more individual branches or fascicles 8. The one or more individual branches or fascicles 8 are each placed within a free tissue graft 10. In certain aspects, the free tissue graft 10 may be an autograft of muscular tissue or dermis tissue previously harvested from a subject. In certain preferred aspects, the free tissue graft 10 is muscle tissue. The free tissue graft 10 is harvested or resected such that it has a standard, predetermined volume or size depending on the size of the branch or fascicle 8. When harvesting the free tissue graft 10, the tissue graft is devascularized and the native blood vessels no longer function. The predetermined volume of the free tissue graft 10 may be selected to be small enough that it is suitably revascularized by collateral blood flow so that the free tissue graft 10 thrives, while providing a sufficiently sized area or volume for the branches or fascicles 8 of the nerve 6 end to grow, as will be described in greater detail below.

Over a period of, for example, several months, the nerve fascicles 8 can reinnervate the free tissue graft 10 and sprout nerve fibers 12 in search of new neural targets. Once the free tissue graft 10 has been reinnervated, the action potentials from neurons traveling down the nerve then generate muscle level signal amplitudes instead of nerve level amplitudes. In this way, the free tissue grafts 10 (e.g., free muscle grafts) act as an amplifier for the signals generated by the branches or fascicles 8 of nerve 6 end, with the signal from a single nerve fascicle 8 having a voltage amplitude of greater than or equal to about 150 μV pp and, in some instances, greater than or equal to about 250 or 500 μV pp and up to, for example, about 1,000 μV pp or more.

While the neural interface system 4 can be used with any lesioned, sectioned, or damaged portion of a nerve (e.g., nerve ending) within a subject, it is particularly suitable for use with peripheral nerves. The neural interface system 4 may thus be used for peripheral nerves suffering damage or injury, such as those involved with amputations. However, the methods described herein may also be used with a variety of different nerves. Thus, in certain aspects, while the methods of the present disclosure are particularly useful with peripheral nerves, the discussion of peripheral nerves and peripheral nerve interface devices is merely exemplary and non-limiting.

As shown in FIG. 1, the free tissue graft 10 can be configured with an electrical conductor, such as an electrode 14, in electrical communication with the free tissue graft 10. The electrode 14, in turn, is in electrical communication with a wire 18a that is in electrical communication with an implant device 20 including processing circuitry 22 and an amplifier 24, as described in further detail below. In such case, the signal from the nerve fascicle 8 is received by the electrode 14 and communicated over the wire 18a to the processing circuitry 22 of the implant device 20 through, for example, the amplifier 24. Alternatively, the electrode 14 can be omitted and the electrical conductor in electrical communication with the free tissue graft 10 can be a wire 18b placed directly in or on the free tissue graft 10. In such case, the signal from the nerve fascicle 8 is received by the wire 18b itself through direct or indirect electrical communication with the free tissue graft 10 and communicated over the wire 18b to the processing circuitry 22 of the implant device 20 through, for example, the amplifier 24. As a further alternative, the electrical conductor in electrical communication with the free tissue graft 10 can be a wire lattice 17 having a multiplicity of electrode sites and a multiplicity of conducting channels that is placed in or on the free tissue graft 10. The wire lattice 17, in turn, is in electrical communication with a plurality of wires 18c that are in electrical communication with the implant device 20. In such case, the signals from the nerve fascicle 8 are received by the wire lattice 17 and communicated over the wires 18c to the processing circuitry 22 of the implant device 20 through, for example, the amplifier 24. The amplifier 24 is a high impedance amplifier. Further, although a single amplifier 24 is shown in FIG. 1, additional amplifiers, including additional / separate amplifier circuits for one or more individual nerve fascicles 8 or groups of fascicles 8 may also be included in the implant device 20. Additionally, multiple implant devices 20 or implant devices with additional processing circuitry 22 may also be used. As referred to herein, the neural interface system 4 may be implantable nerve interface devices, or RPNI devices, which generally include the free tissue graft 10, the associated wires 18a, 18b, 18c, the electrode 14 or wire lattice 17 with multiple electrodes, if any, and the implant device 20 with the processing circuitry 22.

The implant device 20, for example, may be a medical device implantable within a subject, similar to an automatic cardiac defibrillator, but with processing capabilities to receive, process, record, and/or communicate nerve signals received from the free tissue grafts 10, as described in the present disclosure. Because the signals from the individual nerve fascicles 8 are amplified by the free tissue grafts 10 (e.g., free muscle grafts) to levels greater than or equal to, for example, about 150 μV pp or higher, the electronics contained within the implant device 20 are smaller, cheaper, require less processing power, and/or consume less battery power than the electronics that would be needed to sufficiently and meaningfully receive, record, and process nerve signals detected by previous systems, which, as discussed above, are typically less than 100 μV pp when received from within the nerve and less than 10 μV pp when received from a cuff around the nerve. Further, because the signals from the individual nerve fascicles 8 are amplified by the free tissue grafts 10 to levels greater than or equal to, for example, about 150 μV pp or higher, the signals are less susceptible to noise and interference, have higher signal-to-noise ratios, and more precisely represent and correspond to the actual nerve signals produced by the individual nerve fascicles 8. For example, the signals may have a signal-to-noise ratio of 4 or higher. Notably, electrical signals at such levels may be produced by an implantable neural interface system 4 that in certain aspects, consists essentially of a free tissue graft 10 and one or more electrical conductors (e.g., wires 18a, 18b, 18c, electrode 14, and/or wire lattice 17), along with the one or more portions of the nerve 6 that are regenerated and reinnervated in the free tissue graft.

In certain aspects, a method of amplifying a nerve signal in a subject includes disposing a portion (e.g., nerve fascicles 8) of a nerve 6 within a free tissue graft 10 and securing the portion (nerve fascicles 8) of the nerve 6 therein. For example, the free tissue grafts 10 can be attached to the nerve fascicles 8 via sutures, glue, tension, or other suitable attachment methods or mechanisms. Then, at least electrical conductor (e.g., electrode 14, wires 18a, 18b, 18c, and/or wire lattice 17) may be introduced into the free tissue graft 10. It should be noted that the at least one electrical conductor may be introduced into the free tissue graft prior to securing the portion or branch of the nerve to the free tissue graft. The at least one electrical conductor provides electrical communication with the nerve 6. The electrical conductor may have a maximum thickness of less than or equal to about 5 mm. The one or more portions (nerve fascicles 8) of the nerve 6 thus regenerate within the free tissue graft reinnervating the tissue. Such reinnervation may include growing sprout nerve fibers 12. In this manner, the nerve 6 is thus capable of producing an amplified electrical signal of greater than or equal to about 150 microvolts without any external electrical input, as discussed previously above. Notably, the ability to amplify and generate electrical signals from the nerve reflects a voluntary, spontaneous electrical signal generation from the subject at high voltage levels that were previously not possible.

Such a voluntary, spontaneous electrical signal (e.g., generated naturally from motor nerves) can be distinguished from stimulated nerve signals generated by introducing an external electrical input to the nerve for activation (e.g., stimulation by combined compound action potential (CMAPs) resulting from external nerve activation).

In certain other aspects, the method may include cutting a portion of a nerve, such as cutting an ending of the nerve, in the subject to create the one or more branches or fascicles. In certain aspects, the cutting may include cutting the nerve ending into a plurality of portions, like branches/fascicles. Thus, the disposing of the nerve in the free tissue graft and introducing of the electrical conductor into the free tissue graft assembly may be repeated for each respective portion of the nerve. The method may further include harvesting the free tissue graft from a tissue in the subject before the disposing of the cut ending. In certain aspects, the tissue is muscle tissue. In alternative aspects, the tissue may be dermal tissue. As will be discussed in greater detail below, in certain aspects, a maximum dimension of the free tissue graft is less than or equal to about 10 cm. In other aspects, a maximum dimension of the free tissue graft is less than or equal to about 5 cm.

In certain aspects, a method according to certain aspects of the present disclosure may include further stimulating the one or more portions (e.g., branches/fascicles) of the nerve with a stimulus signal delivered through the one or more electrical conductors in electrical communication with the free tissue graft. This provides an ability to deliver sensory feedback via stimulation into the brain of a subject via the neural interface system 4.

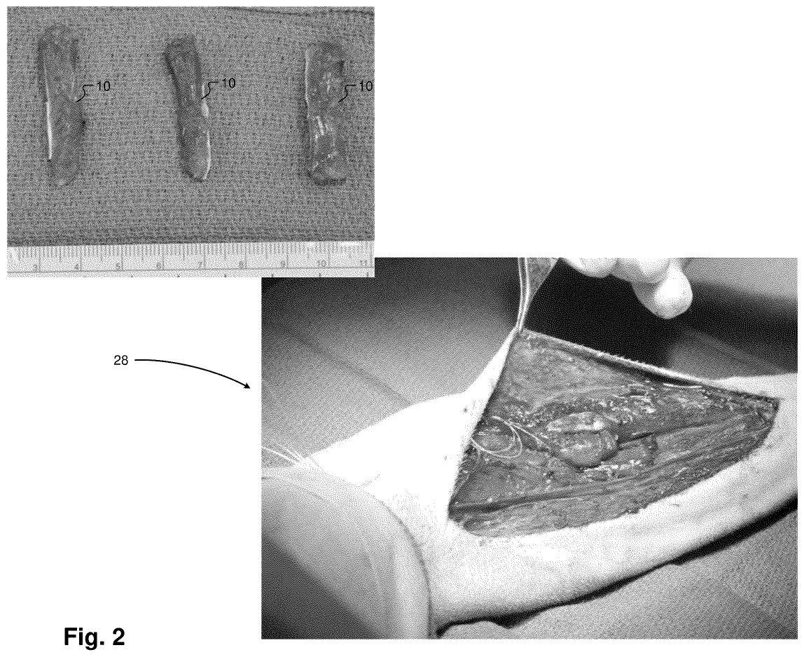

With reference to FIG. 2, three free tissue grafts 10 of free muscle tissue are shown after being harvested from a subject, but prior to being surgically attached to nerve fascicles of the subject. FIG. 2 further includes a photograph 28 showing a surgical procedure for surgically attaching free tissue grafts 10 to nerve fascicles of a subject. Further description for surgically attaching free tissue grafts 10, such as muscle grafts, also referred to as an autograft of a freely grafted piece of autologous muscle tissue from a subject, to a nerve fascicle is provided at commonly assigned U.S. Pub. 2013/0304174, published on Nov. 14, 2013. The entire disclosure of U.S. Pub. 2013/0304174 is incorporated herein by reference.

Because the free tissue grafts 10, e.g., muscle grafts, may be surgically harvested from non-essential donor muscle within the subject, the free tissue grafts 10 undergo a process of complete deinnervation subsequent to being harvested, whereby previously existing innervation within the free tissue grafts 10 terminates. As discussed above, this harvesting process also causes devascularization of the native cells of the free tissue grafts 10. Once the free tissue grafts 10 are surgically attached to nerve fascicles 8, the free tissue grafts 10 undergo a process of reinnervation, whereby the attached nerve fascicles 8 reinnervate the free tissue grafts 10 and sprout nerve fibers 12, which grow within the free tissue grafts 10 in search of new neural targets. Having previously undergone the process of deinnervation, the signals from the newly attached nerve fascicles 8 and newly sprouted nerve fibers 12 do not have to compete with residual nerve signals from the nerve fascicles and nerve fibers that previously innervated the free tissue grafts 10.

Further, instead of simply dying and being reabsorbed by the subject's body, once surgically reattached to the subject, the free tissue grafts 10 can acquire nutrients through a process of imbibition. As such, even without a native vascular blood supply, if the free tissue graft 10 is within an optimal volume/size range, the free tissue graft 10 can absorb nutrients and blood through the surrounding tissue and fluids to support the process of reinnervation. Eventually, a new blood supply network may be established as the free tissue graft 10 reintegrates with the subject's body. This process of deinnervation of the free tissue graft 10 followed by reinnervation of the free tissue graft 10 by the attached nerve fascicle through newly sprouted nerve fibers 12, coupled with the process of imbibition and revascularization, results in an area of muscle or other tissue from which a highly specific electrical signal from an individual nerve fascicle 8 that is greater than or equal to about 150 μV pp or higher, for example, can be received by, for example, the implant device 20.

As mentioned above, to facilitate the processes of reinnervation and imbibition, the free tissue grafts 10 are preferably within an optimal volume/size range. For example, the volume/size of the free tissue graft 10 may be selected to be small enough that it is quickly revascularized by collateral blood flow, while providing a sufficiently sized area or volume for the nerves to grow without forming disorganized neuromas. A greatest dimension of the free tissue graft 10 may be less than or equal to about 10 cm, in certain preferred aspects. For example, in certain variations, the free tissue graft 10 may have a maximum dimension in any direction of less than or equal to about 10 cm. For example, in certain variations, a length of the free tissue graft 10 may be less than or equal to about 10 cm or, more preferably, less than or equal to about 5 cm. Further, a width of the free tissue graft 10 may be less than or equal to about 10 cm or, more preferably, less than or equal to about 5 cm. The thickness of the free tissue graft 10 may optionally be less than or equal to about 2 to 3 cm. Further, optimal dimensions for the free tissue graft 10 may include a length of less than or equal to about 5 cm and a diameter of greater than or equal to about 2 to less than or equal to about 3 cm. For example, preferred optimal dimensions for the free tissue graft 10 may include a length of approximately 3.5 cm and a diameter of approximately 2 cm. It should be noted that the free tissue graft 10 may have a variety of distinct dimensions and/or geometries and those described herein are exemplary. Additionally, a discussion of the dimensions for freely grafted pieces of autologous muscle tissue from a subject is included at, for example, paragraphs [0082] to [0088] of U.S. Pub. No. 2013/0304174, published Nov. 14, 2013, which is incorporated herein by reference in its entirety.

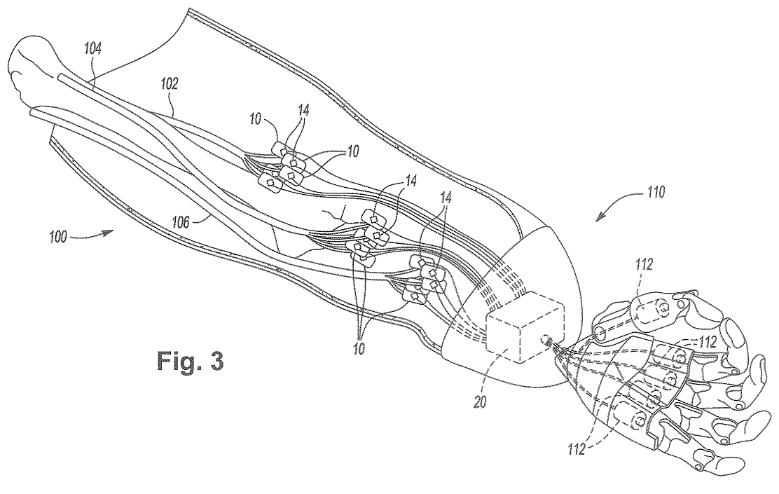

With reference to FIG. 3, an example embodiment 100 is shown with multiple free tissue grafts 10 (e.g., free muscle grafts) and electrodes 14 connected to an implant device 20. In addition to receiving signals from individual nerve fascicles through free tissue grafts 10 and electrodes 14, the implant device also controls a terminal device, in this case a prosthetic hand 110. Specifically, as shown in FIG. 3, each of the radial nerve 102, the median nerve 104, and the ulnar nerve 106 has been split into multiple individual nerve fascicles that have been attached to corresponding free tissue grafts 10 and are in electrical communication with the processing device 22 of the implant device 20 through electrical communication with electrodes 14.

As discussed in further detail below, the processing circuitry 22 of the implant device 20 monitors the signals from the various fascicles and controls, for example, flexion and extension of the prosthetic hand 110 based on analysis of the received signals. For example, as discussed in further detail below, training data can be obtained through a calibration process whereby the subject is asked to perform certain movements while nerve signals are monitored and recorded by the processing circuitry 22 of the implant device 20 and communicated to an external computing device, such as a desktop computer or laptop. The training dataset is then analyzed and used to estimate parameters used by the processing circuitry 22 to drive the prosthetic hand 110, which are then downloaded from the external computing device to the processing circuitry 22 of the implant device 20. For example, as shown in FIG. 3, the implant device 20 is in communication with actuators 112 that drive flexion and extension of individual fingers of the prosthetic hand 110, as described in further detail below.

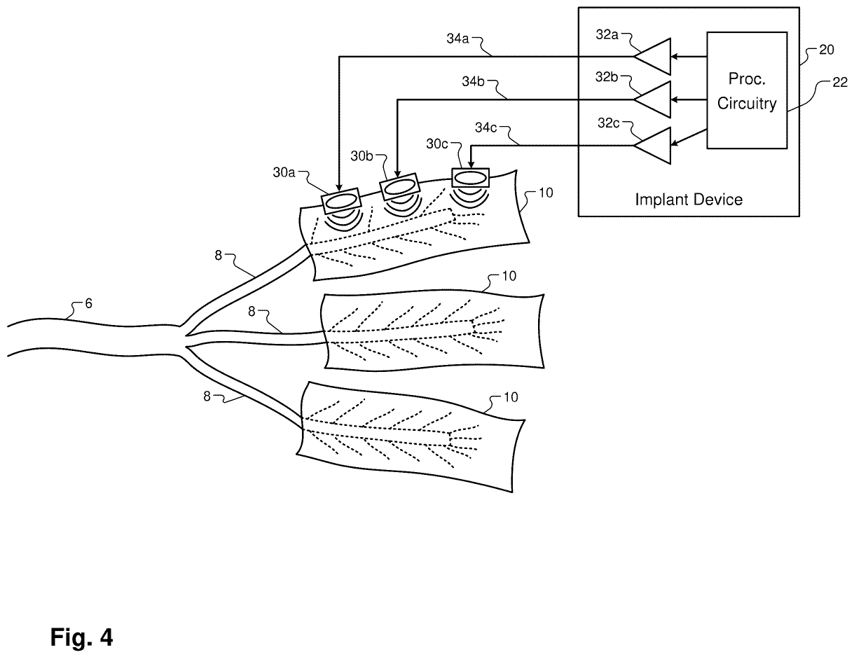

With reference to FIG. 4, in addition to sensing or reading signals generated by nerve fascicles 8 amplified through free tissue grafts 10, the RPNI devices of the present disclosure can also be used for stimulating individual nerve fascicles 8 or individual nerve fibers. For example, as shown in FIG. 4, free tissue graft 10 is configured with three electrodes 30a, 30b, 30c in communication with processing circuitry 22 of the implant device 20 through amplifiers 32, 32b, 32c and wires 34a, 34b, 34c. In this way, as discussed in further detail below, processing circuitry 22 can stimulate individual nerve fascicles 8 using, for example, a negative voltage stimulus signal or a positive voltage inhibitory stimulus signal. Generally, negative voltage signals cause nerves to fire while positive voltages inhibit nerves from firing.

Existing clinical applications, such as vagal nerve stimulation, typically use a cuff around an entire nerve. As such, the majority of the nerve is usually stimulated. Using an RPNI device as shown in FIG. 4, however, the processing circuitry 22 can address stimulation to a particular fascicle by directing signals to electrodes 30a, 30b, 30c through wires 34a, 34b, 34c. More specifically, using a free tissue graft 10 can allow for a single fascicle 8, which may be approximately 1 mm in diameter, to be expanded into a 1 cm by 3.5 cm construct for purposes of stimulation.

Stimulating the entire construct, i.e., the entire free tissue graft 10, can specifically address the single corresponding fascicle 8.

Further, through the use of multiple electrical contacts, such as multiple electrodes, current steering can be used to enable stimulation of an even more specific area, such as a subsection of a fascicle 8 or individual fibers. For example, when utilizing only a single negative contact for stimulation, the negative voltage may spread and dissipate. By using current steering, on the other hand, the negative voltage can be surrounded by positive voltage to focus the negative voltage on a single location. With continued reference to FIG. 4, a negative voltage may be applied with electrode 30b, while positive voltages may be applied with electrodes 30a and 30c in order to provide greater focus on the location for the application of the negative voltage from electrode 30b.

As discussed in further detail below, nerve stimulation can be used for sensory prostheses to stimulate nerves in response to pressure sensed by pressure sensors of a prosthetic limb, for example. Additionally, nerve stimulation can be used to inhibit pathological pain signals. Additionally, nerve stimulation can be used to inhibit pathological contractions of a bladder for example. Additionally, nerve stimulation can be used for sphincter control, erectile dysfunction, and/or to control nerves associated with visceral organs such as the liver, adrenals, stomach, pancreas, kidneys, and the like. For example, such nerve stimulation may be used on a renal artery to disrupt and treat aberrant nerve signals in the kidneys, which may otherwise cause hypertension.

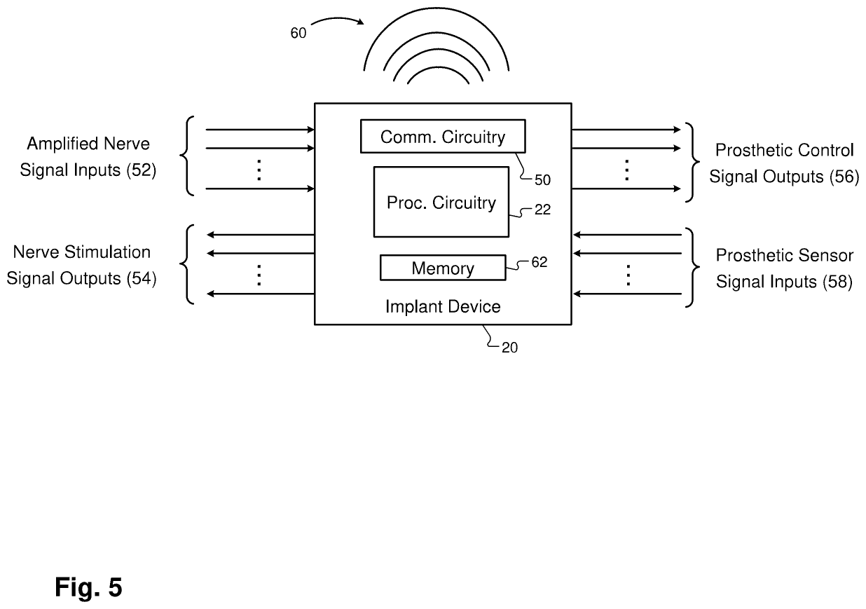

With reference to FIG. 5, further details are shown for the implant device 20. As discussed above, the implant device 20 can receive amplified nerve signal inputs 52 from the free tissue grafts 10 through wires 18a, 18b, 18c (shown in FIG. 1). As further discussed above, the implant device 20 can communicate nerve stimulation signal outputs 54 to stimulate nerves with electrodes 30a, 30b, 30c, through wires 34a, 34b, 34c (shown in FIG. 4). As further discussed above, the implant device 20 can communicate prosthetic control signal outputs 56 to control movements of a prosthetic device. For example, the implant device 20 can communicate prosthetic control signal outputs 56 to control flexion and extension of the prosthetic hand 110 (shown in FIG. 3) via a data bus, such as a controller area network (CAN) bus. For example, the implant device 20 can communicate the prosthetic control signal outputs 56 to control the individual actuators 112 (shown in FIG. 3) of the prosthetic hand.

Further, as further discussed above, implant device 20 can receive prosthetic sensor input signals 58 generated by one or more pressure sensors corresponding to pressure sensed by pressure sensors of a prosthetic limb, for example. As discussed above, implant device 20 can generate nerve stimulation signal outputs 54 based on the pressure sensor signal inputs received by the implant device from the pressure sensors of the prosthetic limb.

As shown in FIG. 5, the implant device includes the processing circuitry 22, as well as communication circuitry 50 and a memory 62, both in communication with the processing circuitry 22. The communication circuitry 50 enables the implant device and, specifically, the processing circuitry 22 of the implant device 20 to wirelessly communicate to computing devices that are external to the implant device 20, including, for example, computing devices that are external to the subject, such as a desktop or laptop computer. In this way, the processing circuitry 22 can communicate data, such as amplified nerve signal input data received via the amplified nerve signal inputs 52. Such communication can be used during a calibration process to receive training and calibration data from the implant device 20 at an external computing device for review and analysis and to communicate estimated operation parameters and configuration data used by the implant device 20 to drive a prosthetic limb, for example, or to generate nerve stimulation signal outputs. The communication circuitry 50 may include an antenna and either a receiver and transmitter or a transceiver for communicating via wireless radio frequency (RF) 60. For example, communication circuitry 50 may communicate via wireless protocols, such as the CEN ISO/IEEE 11073 communication protocol for communication between medical devices and external information system. Alternatively, the communication circuitry 50 may communicate via other wireless protocols, such as WiFi® or Bluetooth®.

The memory 62 can be used by the processing circuitry 22 to store amplified nerve signal input data received via the amplified nerve signal inputs 52 prior to, for example, communication to an external computing device via the communication circuitry 50. The memory 62 can also be used to store estimated operation parameters and configuration data received from an external computing device and used by the implant device during operation. The memory 62 can also be used by the processing circuitry 22 to store event or operation history data, or any other data associated with the various inputs and outputs received or generated by the processing circuitry 22.

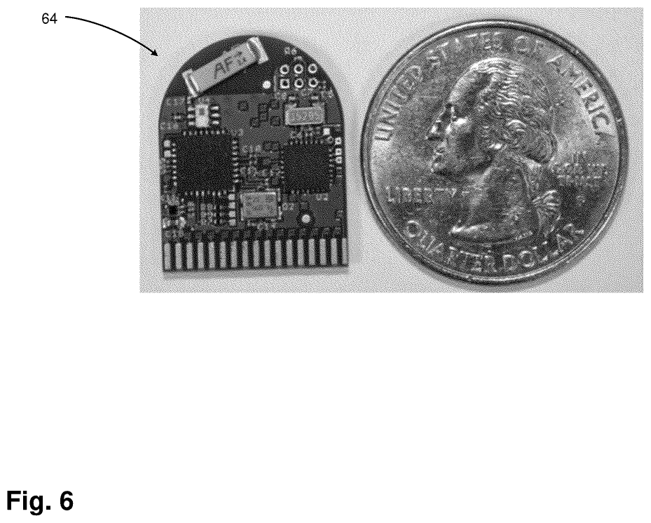

With reference to FIG. 6, a hardware device chip 64 is shown that can include, for example, or be used to implement the processing circuitry 22, communication circuitry 50, and memory 62 of the implant device (shown in FIG. 5). The hardware device chip 64 can also include or be used to implement the amplifier 24 (shown in FIG. 1) and the amplifiers 32a, 32b, 32c (shown in FIG. 4). The hardware device chip 64 can preferably include a high input impedance bioamplifier configured to process the high output impedance biological signals received from the free tissue grafts 10. Further, hardware device chip 64 can preferably include functionality for rejecting large common mode signals, such as recording differentially from paired electrodes, referencing received signals to a local reference near the RPNI device(s), or using a strong high pass filter in a first stage. The hardware device chip 64 can preferably include an amplifier for amplifying the signals received from nerve fascicles 8 by a factor of, for example, 1,000. Preferably, the hardware device chip 64 has very low noise, although this feature may be less important given the relatively high amplitude of the amplified nerve signal inputs received from the nerve fascicles 8. Additionally, the hardware device chip 64 preferably can include a band pass filter to filter received amplified nerve signal inputs between 10,000 to 2,000 Hz before further processing.

Additionally, the hardware device chip 64 preferably can take an absolute value in the analog domain of the received amplified nerve signal inputs.

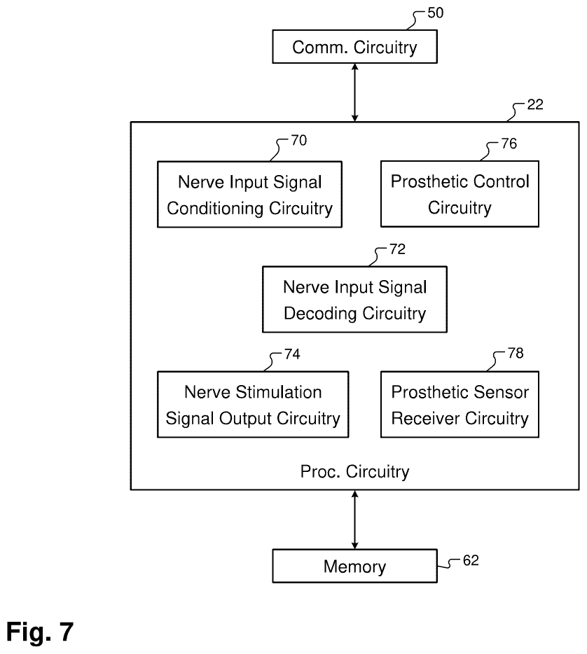

With reference to FIG. 7, further details are shown for the processing circuitry 22, which is shown in communication with the communication circuitry 50 and the memory 62 of the implant device 20. The processing circuitry 22 includes nerve input signal conditioning circuitry 70 for receiving and conditioning the amplified nerve signal inputs 52 received from nerve fascicles through free tissue grafts 10. The functionality and operation of the nerve input signal conditioning circuitry 70 are discussed in further detail below. The processing circuitry 22 also includes a nerve input signal decoding circuitry 72 for processing and decoding the signal data after conditioning by the nerve input signal conditioning circuitry 70. The functionality and operation of the nerve input signal decoding circuitry 72 are discussed in further detail below. The processing circuitry 22 also includes nerve stimulation signal output circuitry 74 for generating nerve stimulation signal outputs 54. The functionality and operation of the nerve stimulation signal output circuitry 74 are discussed in further detail below. The processing circuitry 22 also includes prosthetic control circuitry 76 for generating prosthetic control signal outputs 56 for controlling a prosthetic limb. The functionality and operation of the prosthetic control circuitry 76 are discussed in further detail below. The prosthetic control signal outputs 56 for controlling the prosthetic limb may be communicated to the prosthetic limb via a data bus, such as a CAN bus. The processing circuitry 22 also includes prosthetic sensor receiver circuitry 78 for receiving prosthetic sensor signal inputs 58 from pressure sensors of a prosthetic limb. The functionality and operation of the prosthetic sensor receiver circuitry 78 are discussed in further detail below.

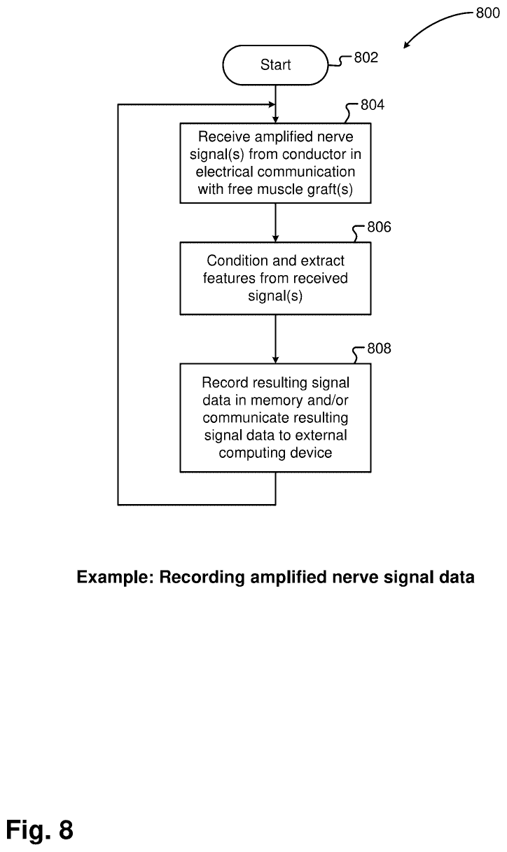

With reference to FIG. 8, a control algorithm 800 is shown for receiving and recording amplified nerve signal data from a free tissue graft 10. The control algorithm 800 may be performed by the processing circuitry 22 of the implant device 20. More specifically, the control algorithm 800 may be performed, at least in part, by the nerve input signal conditioning circuitry 70 (shown in FIG. 7) of the processing circuitry 22. The control algorithm 800 starts at 802.

At 804, the nerve input signal conditioning circuitry 70 of the processing circuitry 22 receives the amplified nerve signal(s) from the conductor in electrical communication with the free muscle graft(s). As described in detail above with reference to FIG. 1, the electrical conductor may be an electrode 14, a wire lattice 17, or a wire 18b in electrical communication with the free tissue graft 10. As further described above, the electrical signal can have a voltage amplitude of greater than or equal to about 150 μV pp and, in some instances, greater than or equal to about 250 or 500 μV pp and up to, for example, about 1,000 μV pp or more. Further, in embodiments where an amplifier 24 (shown in FIG. 1) is used, the signals may be amplified prior to being received by the processing circuitry 22. In such case, for example, the signals may be pre-conditioned by being amplified by a factor of 1,000 with amplifier 24 prior to being received by the nerve input signal conditioning circuitry 70 of the processing circuitry 22.

At 806, the nerve input signal conditioning circuitry 70 of the processing circuitry 22 conditions and extracts features from the received signal from the free tissue graft 10. For example, in embodiments that do not include an amplifier 24 (shown in FIG. 1), the nerve input signal conditioning circuitry 70 may amplify the received signals by, for example, a factor of 1,000. The nerve input signal conditioning circuitry 70 may then filter the received signal using a predetermined analog frequency range. For example, the predetermined analog frequency range may be between 10 and 1,000 Hz or between 10 and 2,000 Hz. After filtering, the nerve input signal conditioning circuitry 70 may digitize the filtered signal using a predetermined sampling rate. For example, the nerve input signal conditioning circuitry 70 may digitize the filtered signal using a sampling rage of 30,000 samples per second. The digitized signal may then be digitally filtered using a predetermined digital frequency range. For example, the predetermined digital frequency range may be 100 to 500 Hz. After digital filtering, the signal may then be down-sampled to 1,000 samples per second. Because of the relatively large voltage amplitude of the initially received signal, the conditioning and feature extraction performed at step 806 results in a robust and well-defined signal that is less susceptible to noise or interference, as compared with systems that do not utilize free tissue grafts 10 for amplification of signals from nerve fascicles.

At 808, the processing circuitry 22 records the resulting signal data in memory 62 and/or communicates the resulting signal data to an external computing device using the communication circuitry 50. For example, the resulting signal data can be stored in the memory 62 of the implant device 20 and then communicated via a batch communication process to an external computing device through the communication circuitry 50. Alternatively, the memory 62 can serve as a buffer that receives and stores the resulting signal data for further processing by the processing circuitry 22 or communication to an external computing device through the communication circuitry 50. Alternatively, the resulting signal data can be streamed to an external computing device in real-time through the communication circuitry 50.

After recording or communicating the resulting signal data at 808, the processing circuitry 22 loops back to 804 and continues to receive amplified nerve signal(s). Although the control algorithm 800 is shown as sequential steps for purposes of illustration, it is understood that the individual steps can occur continually in parallel by the processing circuitry 22 as amplified nerve signals are continually received in real-time.

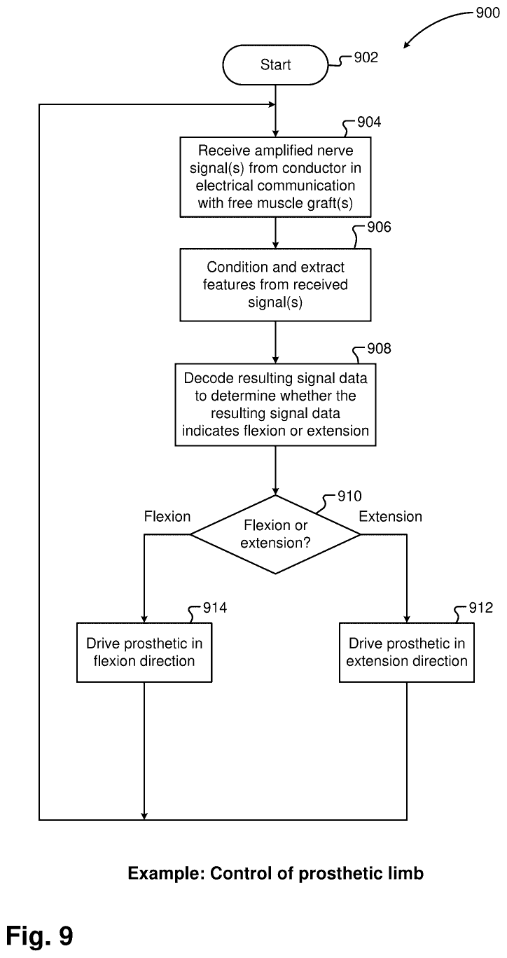

With reference to FIG. 9, a control algorithm 900 is shown for controlling a prosthetic limb, such as a prosthetic hand 110 (shown in FIG. 3) based on signals received from the from the free tissue grafts 10. The control algorithm 900 may be performed by the processing circuitry 22 of the implant device 20. More specifically, the control algorithm 900 may be performed, at least in part, by the nerve input signal conditioning circuitry 70, the nerve input signal decoding circuitry 72, and the prosthetic control circuitry 76 (shown in FIG. 7) of the processing circuitry 22. The control algorithm 900 starts at 902.

At 904, the nerve input signal conditioning circuitry 70 of the processing circuitry 22 receives the amplified nerve signal(s) from the conductor in electrical communication with the free muscle graft(s). The functionality of step 904 is described above with respect to step 804 of FIG. 8 and is not repeated here.

At 906, the nerve input signal conditioning circuitry 70 of the processing circuitry 22 conditions and extracts features from the received signal from the free tissue graft 10. The functionality of step 906 is described above with respect to step 806 of FIG. 8 and is not repeated here.

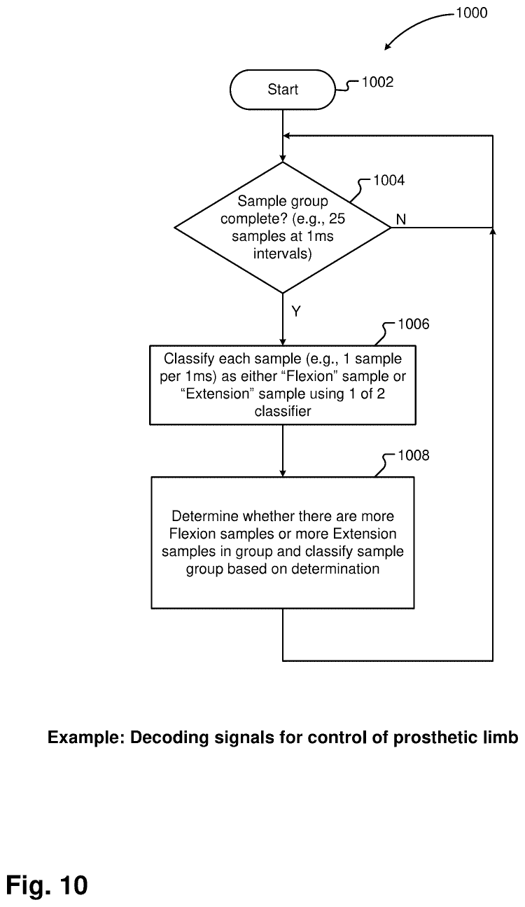

At 908, the nerve input signal decoding circuitry 72 decodes the resulting signal data to determine whether the resulting signal data corresponds to, for example, flexion or extension of the prosthetic limb. While the control algorithm 900 of FIG. 9 is described in terms of decoding the resulting signal data for flexion or extension actions, it is understood that other prosthetic limb control movements could likewise be decoded from the resulting signal data, as appropriate. The nerve input signal decoding circuitry 72 may use a one of two classifier, such as a Naïve Bayes classifier, or regression analysis, to determine whether the resulting signal data over a predetermined time period segment, such as 25 milliseconds (ms), indicates either flexion or extension. Further details for decoding the resulting signal data are described below with respect to the control algorithm 1000 for decoding signals for control of a prosthetic limb shown in FIG. 10.

At 910, the processing circuitry 22 determines whether the resulting signal data for the predetermined time period segment corresponds to either flexion or extension of the prosthetic limb. At 910, when the resulting signal data corresponds to extension, the processing circuitry proceeds to 912 and the prosthetic control circuitry 76 of the processing circuitry 22 drives the prosthetic limb in the extension direction. At 910, when the resulting signal data corresponds to extension, the processing circuitry proceeds to 914 and the prosthetic control circuitry 76 of the processing circuitry 22 drives the prosthetic limb in the flexion direction. For example, in the case of a prosthetic hand 110, as shown in FIG. 3, the processing circuitry 22 can drive actuators 112 of the prosthetic hand 110 in either a flexion or extension direction, as appropriate. After driving the prosthetic limb at steps 912 or 914, the processing circuitry 22 loops back to 904.

With reference to FIG. 10, a control algorithm 1000 is shown for decoding signals for control of a prosthetic limb. The control algorithm 1000 may be performed by the processing circuitry 22 of the implant device 20. More specifically, the control algorithm 1000 may be performed, at least in part, by the nerve input signal decoding circuitry 72. The functionality of control algorithm 1000 shown in FIG. 10 is encapsulated in step 908 of FIG. 9. The control algorithm 1000 starts at 1002.

At 1004, the nerve input signal decoding circuitry 72 determines whether a current sample group for a predetermined time period segment is complete. For example, the predetermined time period segment may be 25 ms and the sample interval may be a 1 ms interval. In such case, the nerve input signal decoding circuitry 72 may wait at step 1004 until a complete sample group of 25 samples at 1 ms intervals is complete. When it is not yet complete, the nerve input signal decoding circuitry 72 loops back to 1004. When it is complete, the nerve input signal decoding circuitry 72 proceeds to 1006.

At 1006, the nerve input signal decoding circuitry 72 classifies each sample in the sample group using a one-of-two classifier. For example, when the nerve input signal decoding circuitry 72 is decoding the resulting signal data for either flexion or extension, the nerve input signal decoding circuitry 72 may classify each sample within the sample group as either a flexion sample or an extension sample. For example, the nerve input signal decoding circuitry 72 may use a one-of-two Naïve Bayes classifier, or regression analysis, to classify each sample within the sample group as either a flexion sample or an extension sample.

The one-of-two Naïve Bayes classifier can use training data collected earlier from the subject during calibration procedures and routines. For example, the subject may be commanded to perform a flexion or an extension action and the resulting nerve signal data can be recorded by the processing circuitry 22 and communicated to an external computing device for analysis. Based on the collected training data, Gaussian distributions can be estimated or computed, based on the received nerve signal data, for each of the flexion and extension movements. For example, the Gaussian distributions for the flexion and extension movements will then have different means and variances. The parameters and data for the one-of-two Naïve Bayes classifier can be estimated based on the collected training data by an external computing device and then communicated to the processing circuitry 22 and stored in memory 62 for use by the nerve input signal decoding circuitry 72 in decoding nerve signal data.

During step 1006, the nerve input signal decoding circuitry 72 can compare each sample within the sample group to the previously determined Gaussian distributions having different means and variances for flexion and extension movements and calculate a probability that the particular sample was drawn from each of the two distributions. Each sample is then classified based on which of the two movements has a higher probability for the particular sample. For example, if a particular sample has a higher probability that it corresponds to a flexion movement, the sample is classified as a flexion sample. If the particular sample has a higher probability of corresponding to an extension movement, the sample is classified as an extension sample. Once all of the samples within the sample group have been classified, the nerve input signal decoding circuitry 72 proceeds to 1008.

At 1008, the nerve input signal decoding circuitry 72 determines whether there are more flexion samples or more extension samples in the particular sample group and classifies the entire sample group based on the determination. For example, when there are more flexion samples in the sample group, the sample group is classified as a flexion sample group and when there are more extension samples in the sample group, the sample group is classified as an extension sample group. In this way, the nerve input signal decoding circuitry 72 predicts whether a group of samples in the particular sample group is indicating a flexion movement or an extension movement, for example. It is understood that other movements may likewise be included in the classification and prediction process. After classifying the sample group, the nerve input signal decoding circuitry 72 loops back to 1004.

In this way, with reference to both FIGS. 9 and 10, the processing circuitry 22 can determine or predict, for example, whether the nerve signal data for a particular predetermined time period segment, such as 25 ms, corresponds to or is indicating a flexion movement or an extension movement. Further, the prosthetic control circuitry 76 can send control commands to the prosthetic limb every 25 ms based on the classification of the current or most recent sample group. In this way, the processing circuitry 22 can continually monitor and decode the nerve signal data and send corresponding commands to operate the prosthetic limb. While the above examples are described using a predetermined time period segment, it is understood that shorter or longer predetermined time period segments can be used.

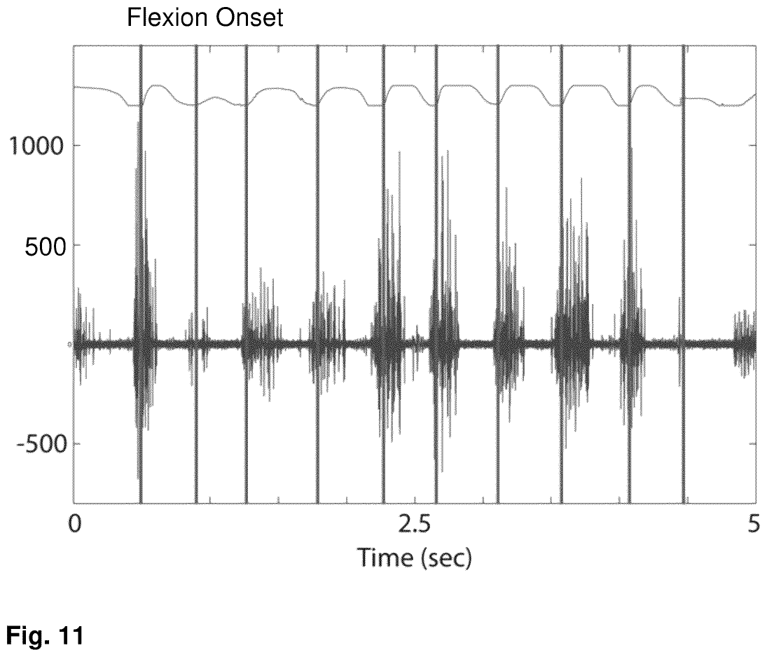

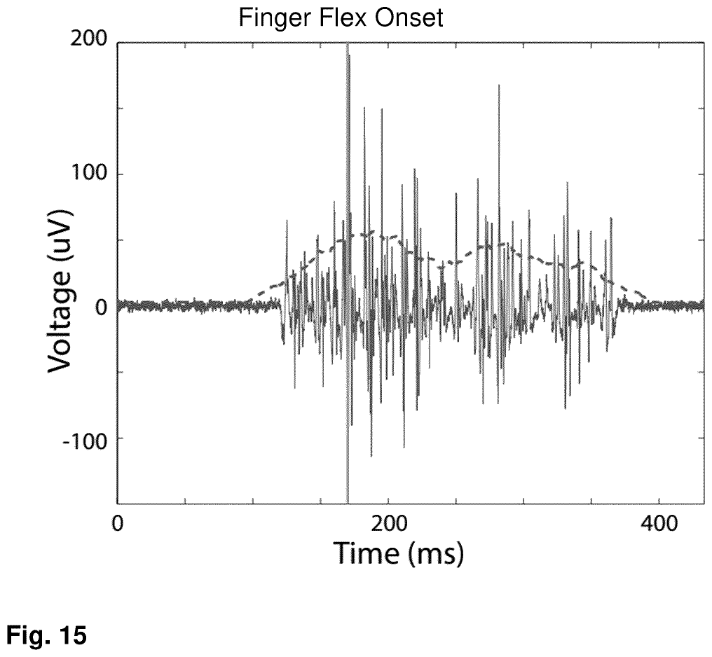

With reference to FIG. 11, an example graph is shown with nerve signal data over a 5 second time period corresponding to onset of a flexion movement. Similarly, with reference to FIG. 15, an example graph is shown with nerve signal data over a 400 millisecond time period corresponding to onset of a finger flexion movement.

As described above with respect to FIGS. 9 and 10, training data collected from the subject during a calibration routine can be used to generate a mapping, for example, of received nerve signals to corresponding prosthetic limb movements or actions. In an example of a transradial amputee, individual nerve fascicles may map well to individual hand muscles simulated with the prosthetic limb. In an example of a transhumeral amputee, the training data can be used to determine which nerve fascicles or sets of nerve signal inputs correlate best to which individual hand muscles simulated with the prosthetic limb.

Additionally, nerve signal data, such as average signal power, number of zero crossing events, or count of detected spikes can also be monitored, recorded, and analyzed for each signal from each nerve fascicles and used to calculate a desired velocity, for example, for all five fingers of a prosthetic hand to send in a single command to the prosthetic hand at each time step, e.g., each 25 ms. There is not a one-to-one correspondence between particular muscles and the velocity of individual fingers. For example, to flex only a pinkie finger, a subject may need to simultaneously extend an index finger. As such, finger velocities can be regressed against muscle activity across all of the nerve signal channels to determine a consistent overall map.

Various algorithms are available to estimate instantaneous velocities from a variety of signals, including, for example, linear filters, Kalman filters, and particle filters.

Additionally, individual discrete states, like grasping and pointing, can be predicted using linear discriminants, Naïve Bayes classifiers, or support vector machines. In each case, a training dataset is obtained through a calibration process by asking the subject to perform a variety of movements or actions and monitoring and recording the resulting nerve signal data using the implant device 20 and processing circuitry 22. The training dataset can then be used to estimate operational parameters used by the processing circuitry 22 and, for example, the prosthetic control circuitry 76, to control the prosthetic hand. The estimated operation parameters can then be downloaded to the processing circuitry 22 through the communication circuitry 50 and stored in memory 62 for use by the processing circuitry 22 to make real-time estimations of finger velocity to drive the prosthetic hand, for example.

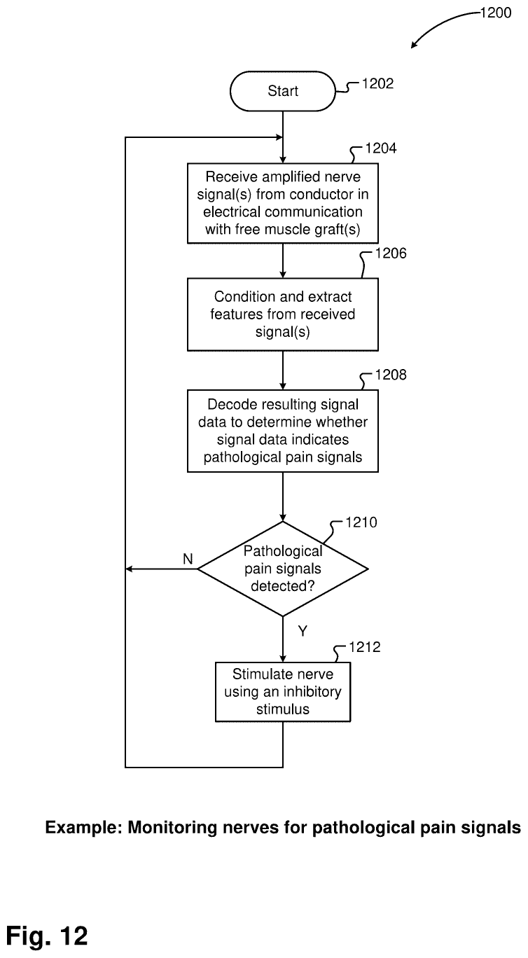

With reference to FIG. 12, a control algorithm 1200 is shown for monitoring nerves for pathological pain signals. The control algorithm 1200 may be performed by the processing circuitry 22 of the implant device. More specifically, the control algorithm 1200 may be performed, at least in part, by the nerve input signal conditioning circuitry 70, the nerve input signal decoding circuitry 72, and the nerve stimulation signal output circuitry 74. The control algorithm 1200 starts at 1202.

At 1204, the nerve input signal conditioning circuitry 70 of the processing circuitry 22 receives the amplified nerve signal(s) from the conductor in electrical communication with the free muscle graft(s). The functionality of step 1204 is described above with respect to step 804 of FIG. 8 and is not repeated here.

At 1206, the nerve input signal conditioning circuitry 70 of the processing circuitry 22 conditions and extracts features from the received signal from the free tissue graft 10. The functionality of step 1206 is described above with respect to step 806 of FIG. 8 and is not repeated here.

At 1208, the nerve input signal decoding circuitry 72 decodes the resulting signal data to determine whether the resulting signal data indicates, for example, pathological pain signals. The decoding performed at 1208 is similar to the decoding described above with respect to step 908 of FIG. 9 and steps 1004 to 1008 of FIG. 10, which describe decoding resulting signal data to determine whether the resulting signal data indicates flexion or extension actions. Like the decoding described above with respect to FIGS. 9 and 10, the decoding performed at 1208 can likewise use a one of two classifier, such as a Naïve Bayes classifier, based on a training data set collected during a calibration procedure with the subject, to determine whether the resulting signal data corresponds to a state where pathological pain signals are being generated or not. After decoding the resulting signal at 1208, the processing circuitry 22 proceeds to 1210.

At 1210, the processing circuitry 22 determines whether pathological pain signals have been detected based on the decoding of the resulting signal data.

When pathological pain signals are detected, the processing circuitry 22 proceeds to 1212 and stimulates the appropriate nerve fascicles with an inhibitory stimulus.

Specifically, the nerve stimulation signal output circuitry 74 of the processing circuitry 22 can stimulate the appropriate nerve fascicle with a positive voltage to inhibit neural activity and inhibit or reduce the pathological pain signal activity in the nerve fascicle. In this way, pain signals within the subject can be mitigated without permanently losing sensation in the particular nerves or nerve fascicles at issue. At 1212, after stimulating the nerve using an inhibitor stimulus, or at 1210 after determining that pathological pain signals have not been detected, the processing circuitry 22 loops back to 1204.

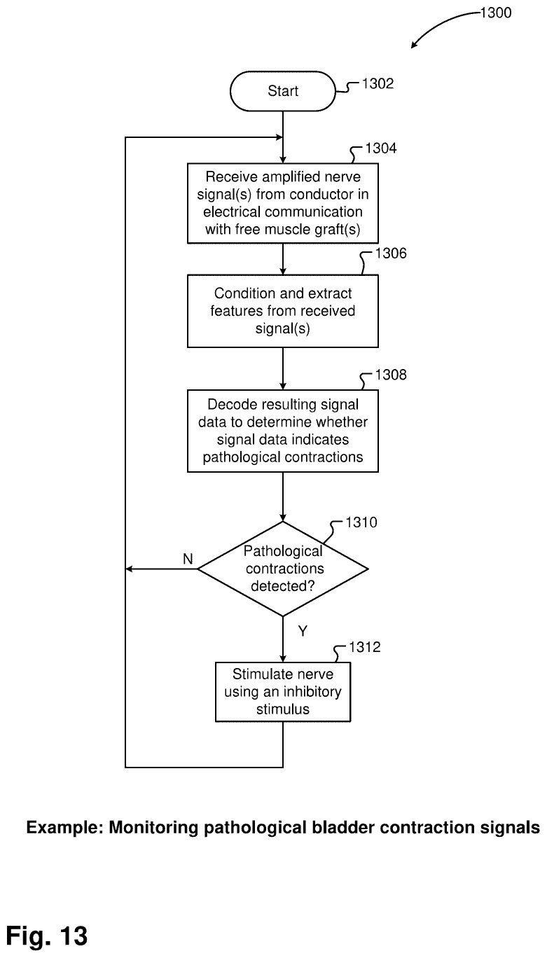

With reference to FIG. 13, a control algorithm 1300 is shown for monitoring pathological bladder contraction signals. The control algorithm 1300 may be performed by the processing circuitry 22 of the implant device. More specifically, the control algorithm 1300 may be performed, at least in part, by the nerve input signal conditioning circuitry 70, the nerve input signal decoding circuitry 72, and the nerve stimulation signal output circuitry 74. The control algorithm 1300 starts at 1302.

At 1304, the nerve input signal conditioning circuitry 70 of the processing circuitry 22 receives the amplified nerve signal(s) from the conductor in electrical communication with the free muscle graft(s). The functionality of step 1304 is described above with respect to step 804 of FIG. 8 and is not repeated here.

At 1306, the nerve input signal conditioning circuitry 70 of the processing circuitry 22 conditions and extracts features from the received signal from the free tissue graft 10. The functionality of step 1306 is described above with respect to step 806 of FIG. 8 and is not repeated here.

At 1308, the nerve input signal decoding circuitry 72 decodes the resulting signal data to determine whether the resulting signal data indicates, for example, pathological bladder contraction signals. The decoding performed at 1308 is similar to the decoding described above with respect to step 908 of FIG. 9 and steps 1004 to 1008 of FIG. 10, which describe decoding resulting signal data to determine whether the resulting signal data indicates flexion or extension actions. Like the decoding described above with respect to FIGS. 9 and 10, the decoding performed at 1308 can likewise use a one of two classifier, such as a Naïve Bayes classifier, based on a training data set collected during a calibration procedure with the subject, to determine whether the resulting signal data corresponds to a state where pathological bladder contraction signals are being generated or not. After decoding the resulting signal at 1308, the processing circuitry 22 proceeds to 1310.

At 1310, the processing circuitry 22 determines whether pathological bladder contraction signals have been detected based on the decoding of the resulting signal data. When pathological bladder contraction signals are detected, the processing circuitry 22 proceeds to 1312 and stimulates the appropriate nerve fascicles with an inhibitory stimulus. Specifically, the nerve stimulation signal output circuitry 74 of the processing circuitry 22 can stimulate the appropriate nerve fascicle with a positive voltage to inhibit neural activity and inhibit or reduce the pathological bladder contraction signal activity in the nerve fascicle. At 1312, after stimulating the nerve using an inhibitor stimulus, or at 1310 after determining that pathological pain signals have not been detected, the processing circuitry 22 loops back to 1304.

Although described in the context of monitoring and inhibiting pathological bladder contraction signals, the control algorithm 1300 described with respect to FIG. 13 can likewise be adapted for other applications. For example, the control algorithm 1300 could be appropriately adapted for sphincter control or erectile dysfunction. Likewise, the control algorithm 1300 could, for example, be adapted to control nerves associated with visceral organs such as the liver, adrenals, stomach, pancreas, and kidneys. In each case, training data is collected and analyzed from the subject to generate appropriate monitoring parameters, which are then downloaded to the implant device 20. The implant device 20 then monitors nerve signal activity to determine whether nerve stimulation is appropriate.

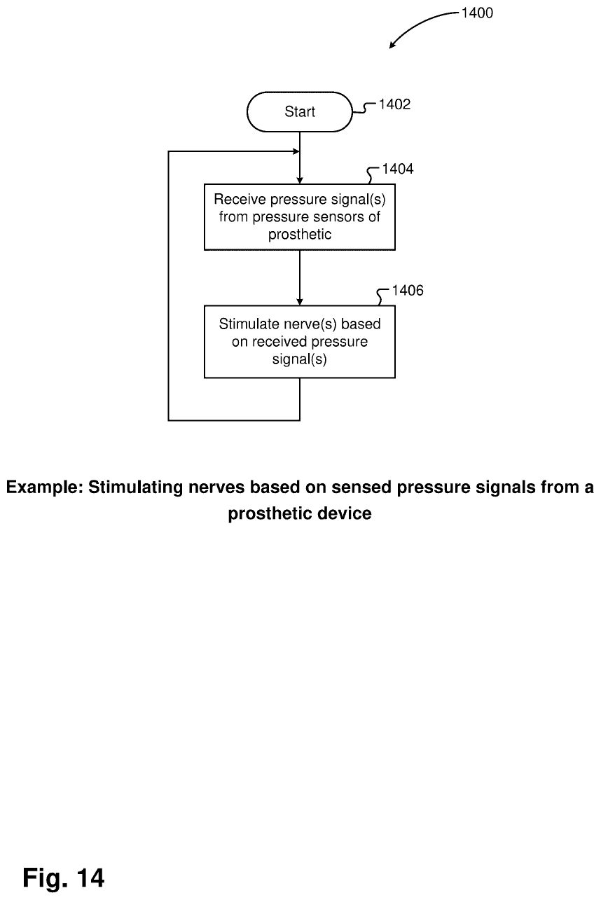

With reference to FIG. 14, a control algorithm 1400 is shown for stimulating nerves based on sensed pressure signals from a prosthetic device. For example, a prosthetic device, such as the prosthetic hand 110 shown in FIG. 3, may be equipped with pressure sensors located, for example, in the fingertips of the prosthetic. The pressure sensors may sense pressure and communicate pressure signals back to the processing circuitry 22 of the implant device 20. The control algorithm 1400 may be performed by the processing circuitry 22 of the implant device. More specifically, the control algorithm 1400 may be performed, at least in part, by the prosthetic sensor receiver circuitry 78 and the nerve stimulation signal output circuitry 74. The control algorithm 1400 starts at 1402.

At 1404, the prosthetic sensor receiver circuitry 78 receives the pressure signals from the pressure sensors of the prosthetic, corresponding to sensed pressure at the location of the pressure sensors. At 1406, the nerve stimulation signal output circuitry 74 stimulates individual nerve fascicles based on the received sensed pressure signals. A calibration procedure with the subject can be used to generate training data to determine which individual nerve fascicles should most appropriately be mapped to which pressure sensors. Further, the firing rate or level of the stimulation can correspond to the level of pressure sensed by the pressure sensors. In this way, the implant device can communicate tactile feedback signals from the prosthetic to the appropriate nerve fascicles.

The following specific examples are provided for illustrative purposes of how to make and use the compositions, devices, and methods of this technology and, unless explicitly stated otherwise, are not intended to be a representation that given embodiments of this technology have, or have not, been made or tested.

### EXAMPLES

### Example 1

### RPNI Studies in Non-Human Primates

In the following example, RPNIs were surgically implanted in the forearms of two nonhuman primates: Monkey R and Monkey L. Specifically, Monkey R had three RPNIs implanted and Monkey L had four RPNIs implanted. Muscle grafts were attached to small branches of the median and radial nerves, providing independent finger flexion/extension and thumb flexion signals. The surgery followed a standard operating procedure checklist and the animals were monitored in-cage daily for ten days post-op and then observed in a primate chair during daily experiments thereafter.

No major complications were noted and the animals regained normal use of the limb within one week after surgery. In a second surgery in both animals, muscle grafts were observed with obvious revascularization. In response to electrical stimulation, the RPNIs produced large amplitude compound muscle action potentials (CMAPs), indicating reinnervation of the muscle grafts by the implanted nerve fascicles.

In a third surgery in Monkey L, four bipolar “IM-MES” intramuscular electrodes manufactured by Ardiem Medical were implanted in the two mature RPNIs and in a healthy, intact muscle (the ECRB, a wrist extensor) for comparison. One electrode was placed in the muscle graft of a newly-created RPNI construct, which subsequently matured over three months to produce high amplitude signals. The presence of the electrode during the maturation phase did not negatively impact the regeneration, reinnervation, and maturation of the RPNI. The electrode leads were tunneled subcutaneously from the monkey's forearm to the back, where they exited percutaneously for connection to recording equipment. Daily recordings from these implanted electrodes were taken during task behavior. The percutaneous site with the exiting leads was lightly cleaned with a betadine solution weekly and no infection was noted. The site appeared clean, with minimal irritation, and did not cause any obvious discomfort for the animal.

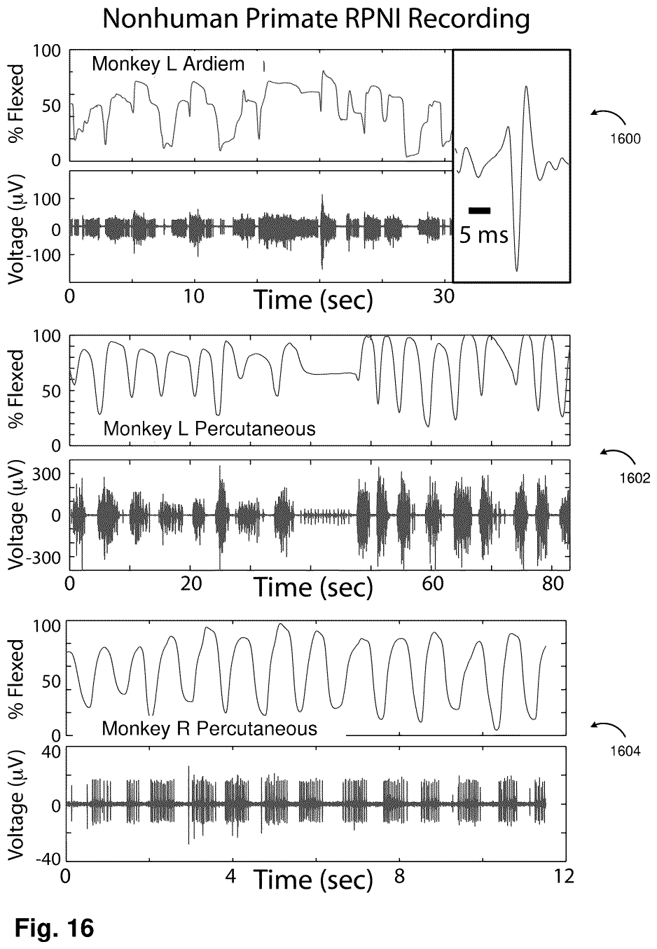

With reference to FIG. 16, graph 1600 shows voluntary RPNI signals, in μV, recorded by semi-chronic Ardiem IM-MES electrodes in Monkey L, along with the calculated flexed percentage corresponding to the RPNI signals. Graph 1602 shows voluntary RPNI signals, in μV, recorded by percutaneous fine-wire electrodes in Monkey L along with the calculated flexed percentage corresponding to the RPNI signals. Graph 1604 shows voluntary RPNI signals, in μV, recorded by percutaneous fine-wire electrodes in Monkey R along with the calculated flexed percentage corresponding to the RPNI signals.

With reference to graph 1600, signals recorded by the IM-MES electrodes vary between animals and RPNI grafts with amplitudes ranging from 50-500 μV pp. The graphs 1600, 1602, 1604 show representative signals, which look similar to sparse electromyographic (EMG) signals, usually displaying multiple apparent single motor units. The rightmost portion of graph 1600 shows a zoomed in portion of the voltage signal showing individual muscle twitches. All putative single units observed correspond reliably to flexion events, are about four ms in length, and have a variable firing frequency. The high signal to noise ratio (SNR) of the RPNI signal allowed an automated detection of voluntary RPNI activation with 95+% accuracy, using a linear discriminant classifier. The RPNI signals were used to control a prosthetic hand in real-time, while Monkey L was performing the behavioral task.

### Example 2

### RPNI Studies in Humans

In the following example, three RPNIs were surgically implanted in a human for the purpose of neuroma control. The patient had a distal transradial amputation just proximal to the wrist. Muscle grafts of approximately 1×3 cm were taken from surrounding tissue and sutured separately onto the distal ends of the median, ulnar, and radial nerves. At this level, the radial nerve (and thus the RPNI graft) contains only sensory fibers which originally innervated the dorsal skin of the hand. The median and ulnar nerves and RPNIs contain a mix of sensory fibers to the hand and motor fibers which originally innervated the intrinsic muscles of the hand.

Electromyographic (EMG) activity was recorded from the median and ulnar RPNIs using percutaneous fine-wire electrodes while the patient performed several hand movements. As expected, RPNIs produced EMG in response to movements which engaged muscles originally innervated by the amputated nerves.

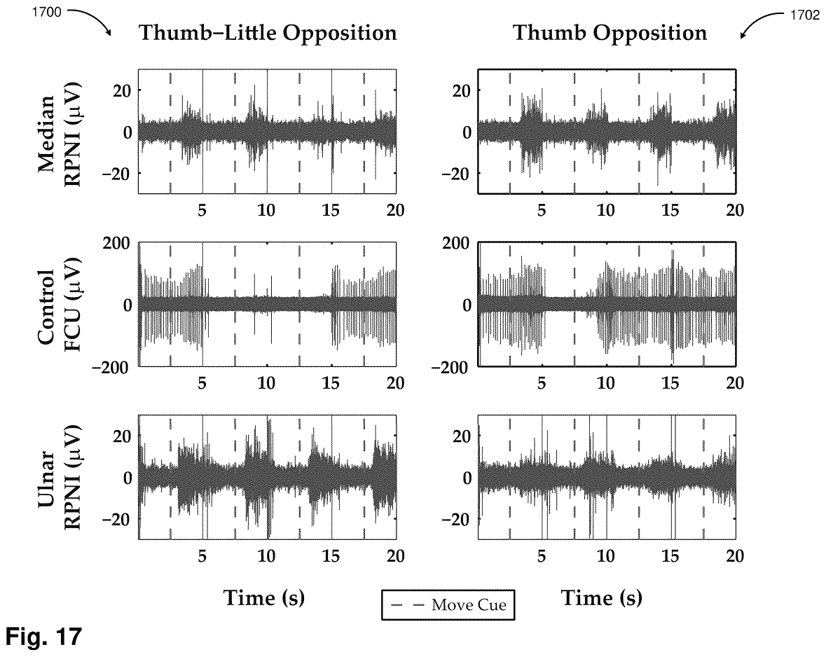

With reference to FIG. 17, graphs 1700 and 1702 show signals recorded from the median-nerve RPNI, the ulnar-nerve RPNI, and a healthy wrist muscle (the Flexor Carpi Ulnaris—FCU) during two different hand movements.

Specifically, graph 1700 shows signals recorded during a thumb-little opposition action, which corresponds to touching the tip of the thumb to the tip of the little finger. Graph 1702 shows signals recorded during a thumb opposition action, which corresponds to touching the tip of the thumb to the base of the little finger, without flexing the little finger. These graphs illustrate that physiologically-correct signals were obtained from the RPNIs. In other words, the nerves are activated during the correct movements.

As expected, the median RPNI signals (shown in the top row of graphs 1700 and 1702) display similar activity amplitudes during both thumb-little opposition and thumb-only opposition. This is because the median nerve originally innervated thumb muscles, but not little finger muscles. The ulnar RPNI signals (shown in the bottom row of graphs 1700 and 1702) are more activated during thumb-little opposition than thumb-only opposition, as the ulnar nerve originally innervated more muscles devoted to the little finger than to the thumb. Finally, the healthy FCU muscle activity, though clearly present, is not correlated to either movement, as it is devoted entirely to flexion of the wrist.

Taken together, graphs 1700 and 1702 show that the RPNIs are being innervated by the expected nerves, as there should be no way to achieve the same pattern of activity via the healthy, intact muscles surrounding the RPNIs.

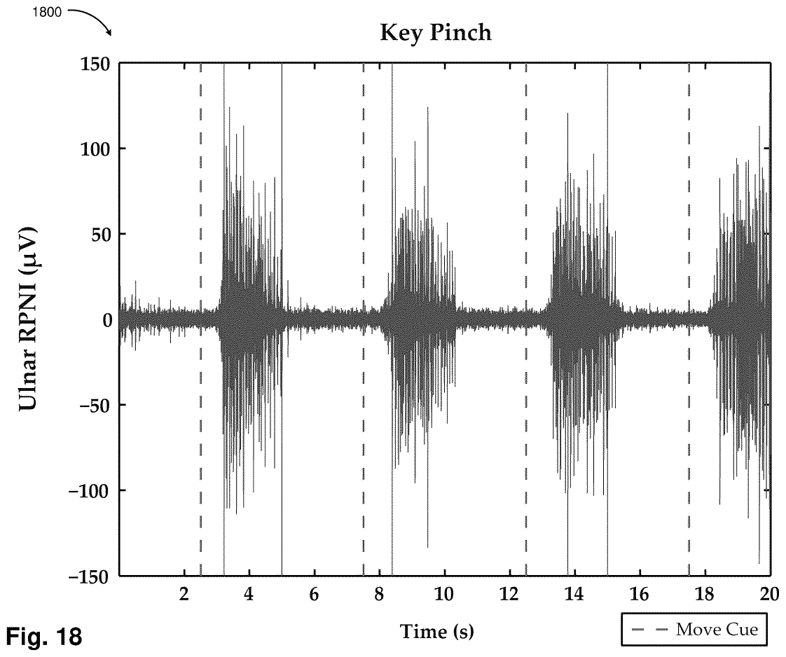

With reference to FIG. 18, graph 1800 shows signals recorded from the ulnar RPNI during a key pinch movement, which corresponds to making a fist with the thumb held against the lateral aspect of the index finger, i.e., the shape made when turning a key in a car ignition. Graph 1800 illustrates the high signal amplitude achievable through the RPNI technique. For example, the signal-to-noise ratio (SNR) of the data in graph 1800 is 8.65.

### Example 3

### Continuous Position Control Studies in Non-Human Primates

In the following example, nerve signals in a monkey were sensed and monitored while the monkey flexed and extended a finger. The nerve signals were processed using the techniques described above with respect to the present teachings, including the use of a Kalman filter, and a percentage of flexion was predicted based on the nerve signals. In addition, the actual percentage of flexion of the monkey's finger was monitored and compared to the predicted percentage of flexion.

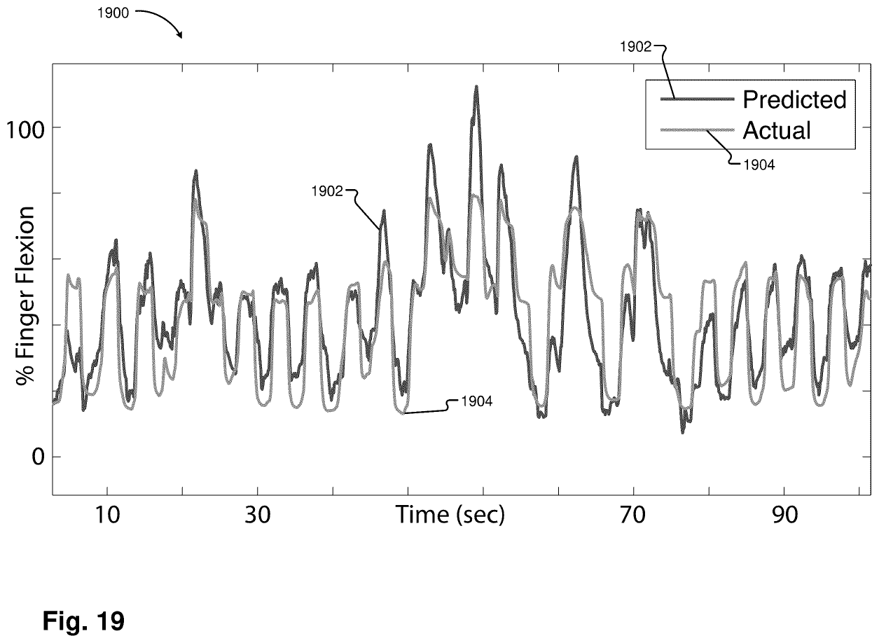

With reference to FIG. 19, graph 1900 shows the predicted percentage of flexion 1902 graphed over time along with the actually observed percentage of flexion in the finger. In graph 1900, the predicted percentage of flexion 1902 correlates to the actual percentage of flexion 1904 by a correlation factor of 0.87. Graph 1900 illustrates the accuracy of the techniques described above with respect to the present teachings, including the use of a Kalman filter, when predicting the actual percentage of flexion based on the monitored nerve signals. In this way, the techniques of the present teachings can be used for continuous position control of a prosthetic. In other words, the present teachings can be used to process nerve signals and control a prosthetic through a range of flexion positions, as opposed to discrete prosthetic positions or states, such as a flexed or extended state.

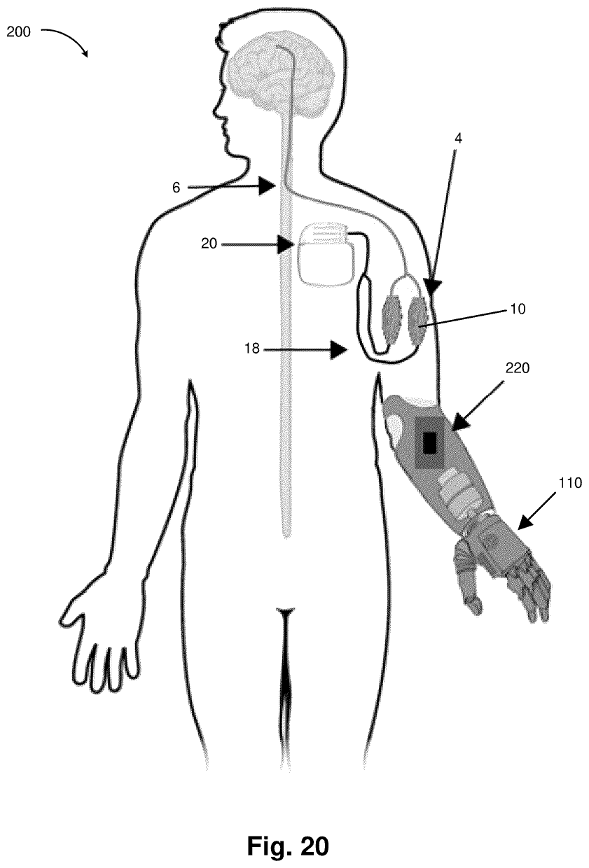

With reference to FIGS. 20 to 30, additional embodiments of the present disclosure are shown and include systems and methods for controlling a prosthetic based on amplified nerve signals. With reference to FIG. 20, a system 200 for controlling a prosthetic device, such as, for example, a prosthetic hand 110, is shown. While the system 200 is described using the example of a prosthetic hand 110, other suitable prosthetic devices can be used. Similar to the systems and methods described above, the system 200 includes a neural interface system 4 with an implant device 20 that receives nerve signals from a nerve 6, such as a peripheral nerve, that have been amplified by free tissue grafts 10, as described above. The signals are communicated from the free tissue grafts 10 to the implant device 20 via electrical leads 18, such as wires 18a, 18b, 18c described above with reference to FIG. 1. The implant device 20, wires 18a, 18b, 18c, free tissue grafts 10, prosthetic hand 110, and neural interface system 4 for amplifying nerve signals from a nerve 6 are described above with, for example, reference to FIGS. 1-7.

The system 200 is similar to the system described above with reference to FIGS. 1-7 except that the implant device 20 of system 200 wirelessly communicates data regarding the amplified nerve signals, such as processed EMG data, to a prosthetic controller 220, as described in further detail below. The prosthetic controller 220 controls actuators 112 (shown in FIG. 3) of the prosthetic hand 110 based on the received signals from the implant device 20 in the same way that the implant device 20 controls the prosthetic hand 110, as described above with reference to FIGS. 1, 3, 5-7, and 9.

In this way, the present disclosure provides systems and methods for controlling a prosthetic hand 110 using amplified nerve signals from free tissue grafts 10. The systems and methods of the present disclosure provide intuitive functional control of multi-articulated prosthetic hands for upper-limb amputation patients who have received the regenerative peripheral nerve interface (RPNI) surgical procedure.

As discussed above, the RPNI surgical procedure places individual small muscle grafts or free tissue grafts 10 on the ends of nerve fascicles in the residual limb. The nerve fascicles reinnervate their respective muscle grafts, forming healthy, stable, and long-lasting neuromuscular connections. Intramuscular bipolar electrodes, further described below, can be used to record directly from RPNIs. While the present disclosure describes example embodiments that utilize RPNIs, the systems and methods of the present disclosure can also be used in with patients who have undergone a targeted muscle reinnervation (TMR) surgical procedure. The TMR procedure includes transferring residual nerves from an amputated limb to reinnervate a new target muscle from the subject that has otherwise lost its function. The reinnervated target muscles then serve as biological amplifiers of the amputated nerve motors signals. Electrodes can then be attached or embedded in the target muscle and connected to the implant device 20 of the present disclosure, which can receive the nerve signals generated by the reinnervated nerves in the target muscle and process the signals in accordance with the present disclosure to control a prosthetic device. In addition, the implant device 20 can communicate signals to the reinnervated nerves in the target muscle to provide sensory feedback stimulation, as described below. In this way, the systems and methods of the present disclosure, including the implant device 20, can be used with patients who have received RPNIs and with patients who have undergone the TMR procedure.

As described above, the device is a fully-implantable recording system that can wirelessly transmit electromyography (EMG) signals to an external device, such as prosthetic controller 220, for upper-extremity prosthetic control. For example, the system can include (1) implantable electrodes and electrical leads 18, (2) an implantable sensing unit, e.g., implant device 20, (3) a wireless transmitter, included, for example, in communication circuitry 50 of the implant device 20, (4) a wireless receiver included, for example, in communication circuitry 222 of the prosthetic controller 220, (5) an external smart link controller, such as prosthetic controller 220, and (6) a charging unit, such as external programmer charger 280 and inductive charging pad 282, which are described in further detail below with reference to FIGS. 20 to 30. For further example, the implant device 20 can be implemented by a modified PMA-approved spinal chord stimulation device (Nuvectra Algovita, PMA P130028). Instead of stimulation, however, the unit can be configured to record EMG signals from both residual muscle and RPNIs using electrode leads. The sensing unit or implant device 20 can wirelessly transmit the signals to an external smart controller, such as the prosthetic controller 220, which will then decode or interpret the EMG signals and send commands to a terminal device, e.g. a myoelectric prosthetic hand, such as prosthetic hand 110. Additionally or alternatively, the implant device can be configured as a stimulation device with a receiver to receive signals from a transmitter of the prosthetic controller 220 and to output signals to the implantable electrodes/electrical leads 18, which are then amplified by the neural interface system 4, to stimulate the nerve 6. In this way, in addition to sensing nerve signals and communicating signals from the implant device to the prosthetic controller, the systems and methods of the present disclosure can also be utilized to wirelessly communicate signals from the prosthetic controller 220 to the implant device 20 that are then used for stimulation of the nerve 6.

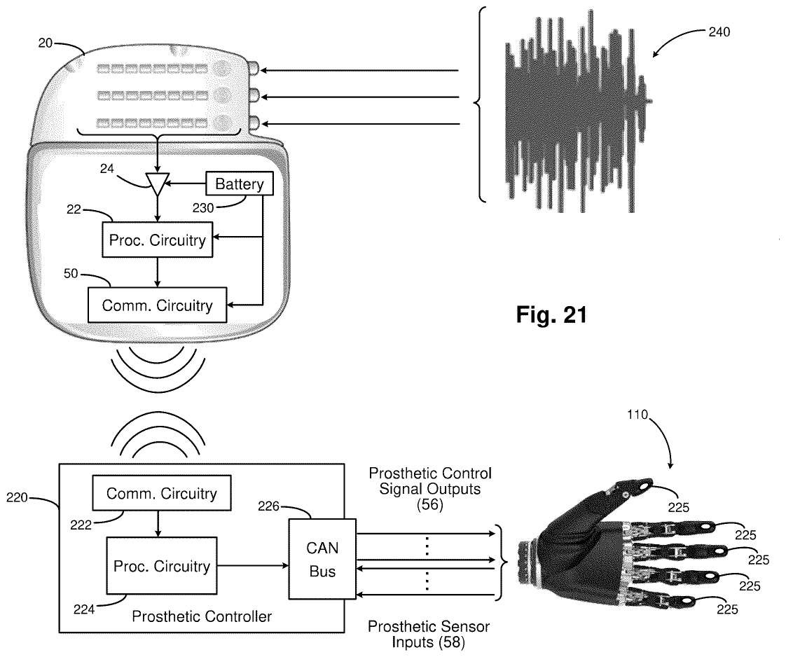

With additional reference to FIG. 21, the implant device 20 includes an amplifier 24, processing circuitry 22, and communication circuitry 50, which are described above. The implant device 20 includes a battery 230 connected to the amplifier 24, the processing circuitry 22, and the communication circuitry 50. While a single amplifier 24 is shown in FIG. 21, additional amplifiers can be used, as discussed below. The implant device 20 receives amplified nerve signals 240 from the free tissue grafts 10 via electrical leads 18. The implant device 20 processes the amplified nerve signals, as described in further detail below, and wirelessly communicates data regarding the amplified nerve signals, such as processed EMG data, via communication circuitry 50 to the prosthetic controller 220. The communication circuitry 50, for example, includes a wireless transmitter configured for wireless communication. The prosthetic controller 220 includes communication circuitry 222 that includes, for example, a wireless receiver configured for wireless communication. Wireless communication between the communication circuitry 50 of the implant device 20 and the communication circuitry 222 of the prosthetic controller 220 can be performed using the Medical Implant Communication Service (MICS) protocol and/or the Medical Device Radiocommunication Service (MedRadio) protocol.

Additionally or alternatively, any other suitable wireless communication protocol can be used, including, for example, Bluetooth, Bluetooth Low Energy (BLE), WiFi, near-field communication (NFC), or other suitable wireless communication protocols.

The processing circuitry 224 of the prosthetic controller 220 is configured to process the signals received by the communication circuitry 222 from the implant device 20 and to communicate prosthetic control signal outputs 56 to control actuators 112 (shown in FIG. 3) of the prosthetic hand 110 via a data communication bus, such as a CAN bus 226. For example, the processing circuitry 224 of the prosthetic controller 220 can perform the control algorithm 900, described above with reference to FIG. 9, for controlling a prosthetic limb based on signals received from free tissue grafts 10. Additionally or alternatively, as discussed above, the prosthetic controller 220 is further configured to process stimulation feedback signal inputs, such as prosthetic sensor input signals 58 generated by one or more pressure sensors 225 corresponding to pressure sensed by pressure sensors 225 of the prosthetic hand 110. For example, as the prosthetic hand 110 grasps an object, the pressure sensors 225 detect pressure generated from grasping the object. The prosthetic sensor input signals 58 can be received via the data communication bus, such as the CAN bus 226, and processed by the processing circuitry 224 of the prosthetic controller 220, which can then communicate stimulation/sensory feedback signals to the implant device 20 via wireless communication transmitted from communication circuitry 222 to communication circuitry 50.

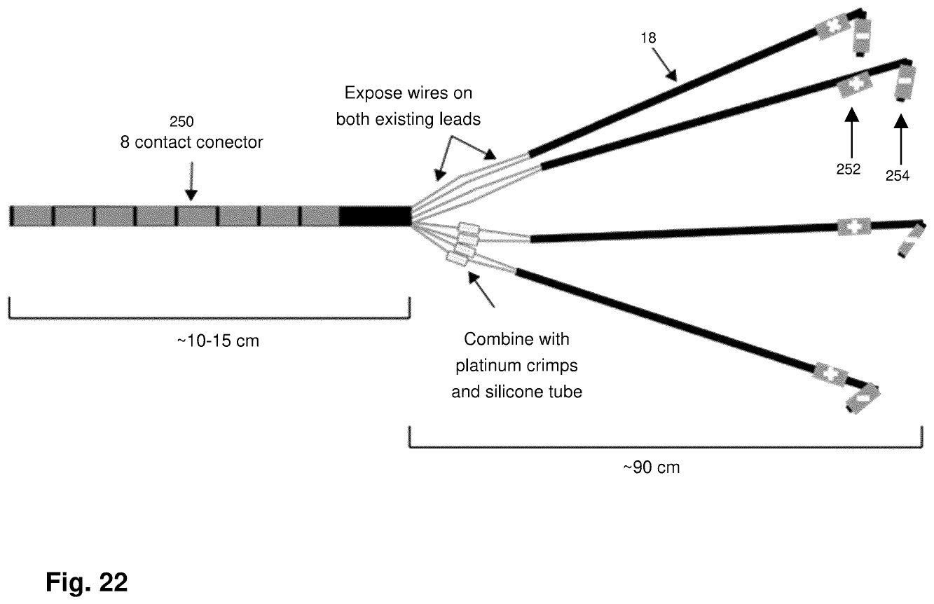

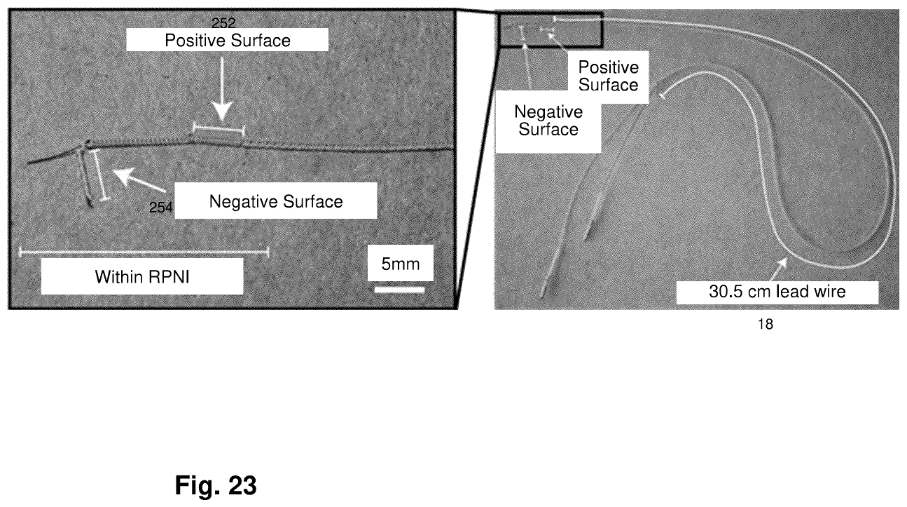

With reference to FIG. 22, four electrical leads 18 are shown, with each electrical lead 18 including two wires connected to a positive electrode 252 and a negative electrode 254, respectively. FIG. 23 shows photographs of the electrical leads 18, the positive surface of the positive electrode 252, and the negative surface of the negative electrode 254. In the example of FIG. 22, the eight wires of the electrical leads 18 are connected to an eight-contact connector 250. In particular, eight contact wires of the eight-contact connector 250 are connected to exposed wires of the electrical leads 18 with, for example, platinum crimps that are then sealed with silicone tubing, as shown in FIG. 22. While the example of FIG. 22 utilizes platinum crimps with silicone tubing to connect the contact wires of the eight-contact connector 250 with the exposed wires of the electrical leads 18, any other suitable method for connecting the contact wires of the eight-contact connector 250 with the exposed wires of the electrical leads 18 can be used. Alternatively, exposed wire of the electrical leads 18 can be connected directly to the contacts of the eight-contact connector 250. In addition, while the example of FIG. 22 utilizes four two-wire electrical leads 18 and an eight-contact connector 250, any other suitable number of wires, electrical leads, and contact connectors can be used. For example, the contact connector can include contacts to accommodate two, four, six, ten, twelve, or any other suitable number of electrical wires, and the electrical leads can include two, four, six, ten, twelve, or any other suitable number of electrical wires.

To record EMG signals from residual muscle and RPNIs, the system 200 utilizes an electrode and lead assembly that connects the muscle tissue, such as the free tissue grafts 10, to the implant device 20. The assembly can include a modified version of an existing bipolar electrode, and a modified version of an existing cable lead that connects to the implant device 20. Each implant device 20, for example, can connect to twelve bipolar electrodes with each lead containing eight contacts with three leads per implant device 20. The bipolar electrodes can be a modified from a PermaLoc™ electrode, which has been approved by the FDA as part of the long-term implanted system NeuRX Diaphragm Pacing System. Compared to a monopolar PermaLoc™ electrode, the bipolar electrode has an additional de-insulated lead surface and the plastic tined anchor at the distal tip of the electrode is removed, as shown in FIG. 23. The basic design, all material parts, and sterilization protocol remains the same. The electrodes can be percutaneously implanted, for example, in patients with upper-extremity amputation to record EMG signals from residual muscles and RPNIs.

For the bipolar electrodes to be fully implanted and connected with the implant device 20, several modifications and additions were made. To connect to the implant device 20, an industry standard Bal Seal connector lead fabricated by Cirtec Biomedical, Inc. was used. A single Bal Seal connector lead has eight contacts with eight conductors parallel to the lead body. Other numbers of contacts, such as twelve conductors, can be used. The lead diameter is 0.053 mm with an estimated impedance to be 4 Ohms per foot. The conductor material used is MP35N with 28% silver, and the outer insulation is 55D Pellethane, with an ETFE conductor insulation. The electrical connection to the Bal Seal uses platinum-iridium connector rings. The bare cables are exposed 10 to 15 cm from the proximal end of the Bal Seal connector lead and expose the bare cables at the proximal end of the PermaLoc bipolar lead. The distal and proximal ends of the two leads are permanently joined to form a single lead with 4 bipolar electrodes. The mating uses platinum crimps to connect each individual bare wire and will be covered with silicone tubing for protection. The protected joint is located as close to the implant device 20 as possible to avoid crossing anatomical joints in the body of the patient with the thicker portion of the lead. The total length of the assembled lead from proximal end to distal end is approximately 100 to 105 cm. As such, the distal and proximal end of two previously approved electrode leads are permanently mated without changing electrical or material characteristics of the leads.

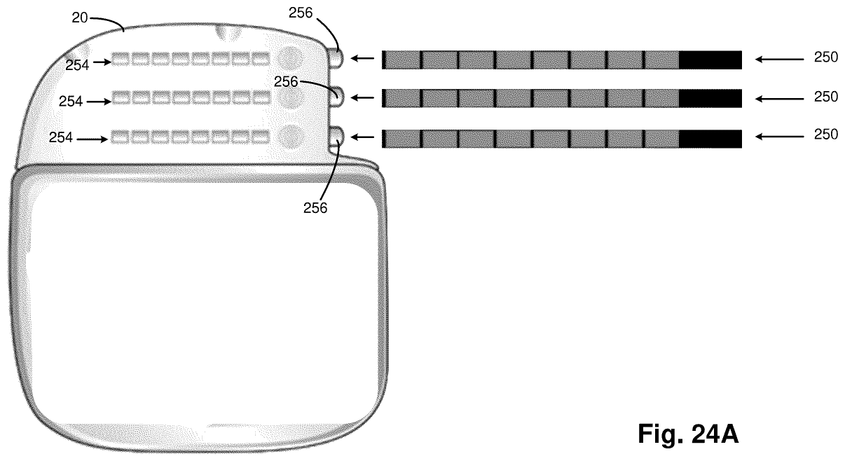

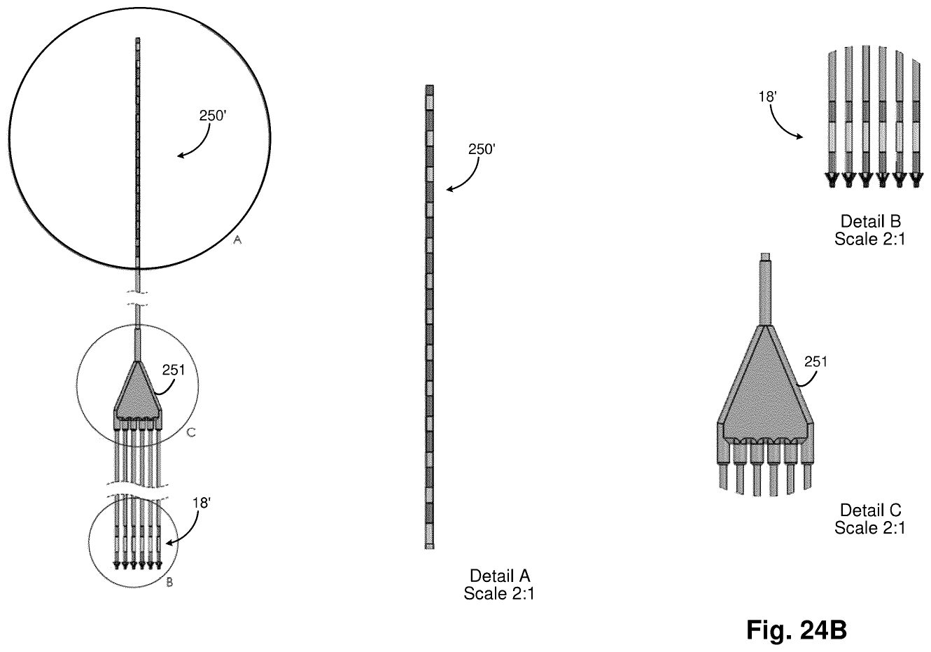

With reference to FIG. 24A, the implant device 20 includes a header that receives one or more contact connectors connected to the electrical leads. For example, as shown in FIG. 24A, the implant device includes electrical ports 256, each configured to receive an eight-contact connector 250. The ports 256 can each include a Bal Seal connector, such as a Bal Seal connector manufactured by Cirtec Biomedical, Inc., configured to seal the implant device 20 once the eight-contact connectors 250 are inserted into the electrical ports. The implant device also includes a header having parallel conductors 254 with contacts that are located and spaced to correspond to and line up with the contacts of the eight-contact connectors 250. In this example, the Baal Seal conductors 254 are manufactured by Bal Seal Inc., and are components of the header manufactured by Cirtec. In the example of FIG. 24A, the implant device is configured with a header having three parallel conductors 254 to receive three eight-contact connectors 250. In this way, the implant device 20 shown in FIG. 24A can accommodate signals from up to 24 wires, i.e., signals from three sets of eight electrical wires based on signals from 24 electrodes including twelve positive electrodes 252 and twelve negative electrodes 254. The twelve positive electrodes 252 and twelve negative electrodes 254 enable the implant device 20 to receive twelve bipolar channels of EMG activity based on the amplified nerve signals 240 from the free tissue grafts 10. While the Example of FIG. 24A utilizes 24 wires and electrodes, the implant device 20 can be configured with a header and with parallel conductors 254 to accommodate any number of wires and electrodes. For example, the implant device 20 can include a header with two sets of parallel conductors 254 that each include 12 contacts to receive twelve-contact connectors. Alternatively, the implant device 20 can include four sets of parallel conductors 254 to receive four eight-contact connectors 250 so that signals from four sets of electrical wires based on signals from 32 electrodes, including 16 positive electrodes 252 and 16 negative electrodes 254 can be received. In this way, the implant device 20 can include a header with any suitable number of parallel conductors 254 with any number of contacts to receive contact connectors with any number of wires.

The implant device 20 can be implemented using a modified implantable recording device based on the Algovita platform developed by Cirtec Biomedical, Inc. The Algovita platform (PMA P130028) is an FDA-approved implantable pulse generator for the reduction of pain via spinal cord stimulation. The electrode leads are connected to the implant device 20 with a header that has 3 rows, each containing 8 spring platinum iridium Bal Seals. The leads are secured after implantation with a titanium setscrew which is tightened with a provided torque wrench inserted through a septum. The Algovita is internally powered by a 4.1 V lithium-ion battery with a nominal capacity of 215 mAh. It has been tested to maintain capacity after 1000 discharge cycles. It also has a low self-discharge rate, so shelf-life is not a concern for this application since the battery exceeds the shelf-life of the sterile packaging. The implant device 20 wirelessly communicates with external components over the medical implant communication system (MICS) with communication circuitry 50 that can include, for example, a Microsemi ZL70323MNJ, a newer version of the MICS chip used in the original Algovita. MICS communication uses a 2.45 GHz band for wake-up and a 402 to 405 MHz band for data transfer. Except for the updated MICS chip, the battery, power management, and communication circuitry all remain unmodified.

The external physical design of the implant device 20 remains the same as the Algovita device. Internal component changes to convert the Algovita to a sensing device are limited. The Saturn stimulator ASIC was replaced with an Intan RHD2216 amplifier for sensing biopotentials instead of stimulating neural tissue. Intan amplifiers have low power consumption and have previously been used in surgically invasive clinical research. No adverse advents were noted in these acute studies. As an example, a linear voltage regulator (TPS7A2033, Texas Instruments) can take the 4.1 V battery voltage as an input and convert it to the 3.3 V signal to power the RHD2216. The (ZL70321MNJ, Microsemi) MICS module on the Algovita was replaced with the newer version ZL70323MNJ from Microsemi.

With reference to FIG. 24B, another embodiment is shown with an alternative arrangement that includes six electrical leads 18′ for six bipolar electrodes connected to a twelve-contact connector 250′. The twelve-contact connector 250′ can be received by a header of an implant device 20, similar to the configuration described above with respect to FIGS. 22-24A. As described above, a port 256 of the implant device 20 can include a Bal Seal connector, such as a Bal Seal connector manufactured by Cirtec Biomedical, Inc., configured to seal the implant device 20 once the twelve-contact connector 250′ is inserted into the electrical port of the implant device 20. The contacts of the twelve-contact connector 250′ are sufficiently spaced to match the spacing of corresponding connectors of the header of the implant device 20. Additionally, in the embodiment of FIG. 24, a triangular coupling device 251 is utilized to receive the six electrical leads 18′ and couple the six electrical leads 18′ to the twelve-contact connector 250′. As shown in FIG. 24B, the coupling device 251 includes parallel connections for the six electrical leads 18′ on the wide part of the triangular coupling device 251 an couples the twelve wires for the six bipolar electrodes to a single cable with each of the twelve wires connected to individual contacts of the twelve-contact connector 250′.

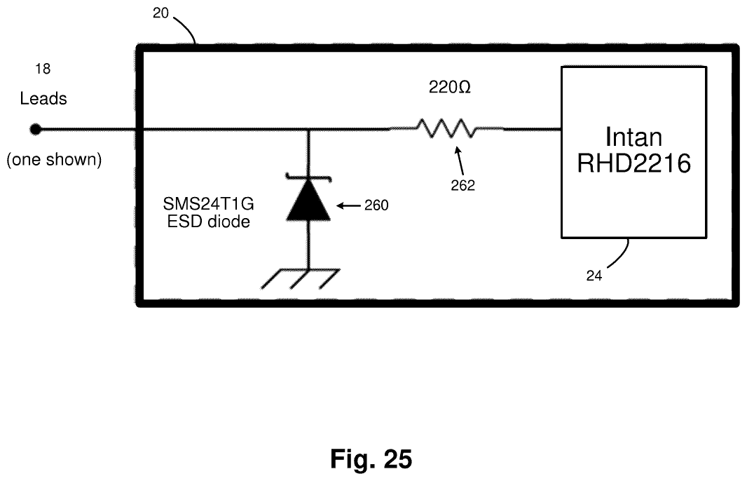

With reference to FIG. 25, the implant device 20 includes protection circuitry to protect electrical components of the implant device from surge voltages during an electrostatic discharge (ESD) event. For example, the implant device may experience an ESD event while the implant device 20 is handled during a surgical procedure to implant the implant device 20 within the body of patient. As shown in FIG. 25, for example, a 220 Ohm resistor 262 is located before the input to amplifier 24 to provide protection for surge voltages that could build up in the case of an ESD event. As an example, the 220 Ohm resistor 262 can be an AC0603JR-07220RL resistor available from Yageo. For further example, as shown in FIG. 25, the amplifier 24 can be an Intan RHD2216 amplifier for sensing biopotentials. In addition, for further example, an ESD diode 260 can be located on the opposite side of the resistor 262 from the input to the amplifier 24. The diode 260, for example, can be an SMS24T1G ESD diode, available from On Semiconductor. While the ESD protection circuitry is illustrated in FIG. 25 for one input to the implant device 20, the same protection circuitry, including the 220 Ohm resistor 262 and the ESD diode 260, can be included for each input to the implant device 20. In this way, for an implant device 20 configured with a header having three sets of contactors 254 for three sets of eight-contact connectors 250, i.e., a total of 24 inputs, as shown in FIGS. 21 to 24, the implant device 20 can include 24 sets of the protection circuitry shown in FIG. 25. In other words, the implant device 20 can include one 220 Ohm resistor 262 and one ESD diode 260 for each of the 2 in puts to the implant device 20. While specific examples of the resistor 262 and diode 260 are shown in FIG. 25, any other suitable resistors and diodes can be used for the protection circuitry to protect the implant device 20 from an ESD event.

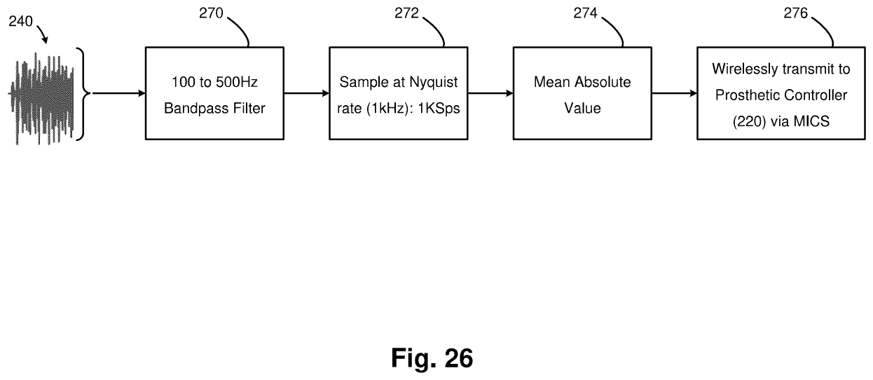

With reference to FIG. 26, a functional block diagram for systems and methods of processing amplified nerve signals in accordance with the present disclosure is illustrated. As shown in FIG. 26, the raw amplified nerve signals 240 are received by the implant device 20. For example, as shown in FIGS. 21 to 24 and as discussed above, the implant device 20 receives twelve bipolar channels of EMG activity from the implanted electrodes and the electrical leads 18. At 270, the amplified nerve signals are filtered using a 100 to 500Hz bandpass filter, which has been shown to provide accurate measurement of EMG activity. At 272, the filtered signals are then sampled by the processing circuitry 22 of the implant device 20 at the Nyquist rate of approximately 1 kHz, resulting in approximately 1 kila-sample per second (1 KSps) of EMG data. At 274, the processing circuitry 22 of the implant device 20 then computes the mean-absolute-value (MAV) of each bipolar channel's activity. At 276, the processing circuitry 22 uses the communication circuitry 50 of the implant device 20 to wirelessly transmit the MAV of each bipolar channel's activity to the communication circuitry 222 of the prosthetic controller 220 using the MICS protocol.

To transmit and receive data wirelessly to and from non-implanted devices, such as the prosthetic controller 220 or another external communication device, the implant device 20 can use communication circuitry 50 of the existing Microsemi MICS communication device from the Algovita platform, as discussed above. Following implantation, the implant device 20 can be turned on/off by waving a magnet over the device. When the implant device 20 is in operation, it can interact with two external devices: the programmer charger, discussed below, and the prosthetic controller 220, also referred to as a Smart Link Controller (SLC). The implant device 20 is configured to wirelessly stream EMG to the prosthetic controller 220, as discussed above. The implant device 20 includes a bootloader for wireless firmware updates. The microcontroller of the implant device 20 is the Texas Instruments MSP430F2618 (MSP430), also used in the Algovita platform. The microcontroller is programmed to record 12 bipolar channels of EMG activity from the implanted electrodes using the Intan RHD2216 amplifier.

In this way, instead of sending the raw EMG data to the prosthetic controller 220, the implant device 20 first filters, samples, and processes the raw EMG signals from the bipolar channels to compute the MAV of the sampled EMG signals for each bipolar channel before and then wirelessly transmits only the processed MAV data for each bipolar channel to the prosthetic controller 220. This configuration provides the technological advantage of maximizing battery life for the battery 230 of the implant device 20 and for the power source of the prosthetic itself as it would require much more power to stream the higher bandwidth raw EMG signals directly from the implant device 20 to the prosthetic controller 220. In this way, the systems and methods of the present disclosure maximize the battery life of the devices by first filtering, sampling, and processing the raw EMG data at the implant device 20 and then wirelessly transmitting only the processed EMG signals for each bipolar channel to the prosthetic controller 220.

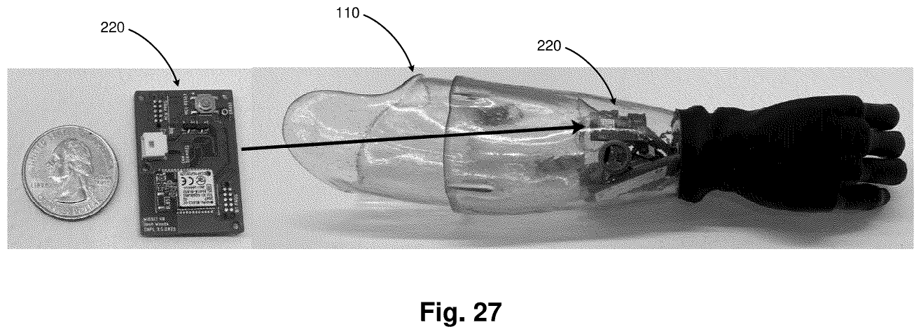

With reference to FIG. 27, an example of a prosthetic controller 220 is shown both outside of a prosthetic hand 110 and installed within the prosthetic hand 110. A quarter is also shown in FIG. 27 to illustrate the relative size and scale of the prosthetic controller 220. As shown in FIG. 27, the prosthetic controller 220 can be implemented using a printed circuit board assembly (PCBA).

The prosthetic controller 220 receives the processed EMG packets and decodes them into movement commands for the prostheses. The prosthetic controller 220, for example, includes communication circuitry 222 that can include, for example, a ZL70123 chip to communicate wirelessly and securely with the implant device 20 using the standard MICS protocol. All components of the prosthetic controller 220 are soldered on the PCBA and the device is enclosed in a water-resistant housing within the prosthetic. The materials used in the prosthetic controller 220 are typical for modern PCBAs. The circuit board is a flat laminated composite with internal copper circuitry. The components mounted on the circuit board are application specific integrated circuits (ASICS) being used as intended by the manufacturer. ASICS are attached to the board with a lead-free solder with industry standard surface-mount technology.

The prosthetic controller uses state-of-the art algorithms, including machine learning algorithms, to decode EMG signals into movement commands, as described above. The choice of algorithm depends on the operation mode of the prosthetic hand. A classifier may be used to decode grasps for intuitive grip selection, with additional gains for proportional control. Alternatively, a regressor may be used to simultaneously and independently control individual fingers.

The prosthetic controller can be connected to a laptop, such as an external programmer device 290 described below, to record processed EMG from the implant device 20. The implant device 20 can also be wirelessly connected to, and communicate with, the laptop, such as external programmer device 290, as discussed below. Recorded EMG can be used to measure the signal strength from each electrode pair and calculate decoding parameters. A configuration mode can be used to load new parameters onto the prosthetic controller 220. A test mode is available to verify prosthetic controller functionality of the implant device 20. In test mode, the external programmer device 290 can transfer pre-recorded EMG packets to the prosthetic controller, which will respond with decoded movement commands. Otherwise, the device operation is fixed and includes: taking in signals, decoding intended movements, and sending outputs to the prosthesis.

The power for the prosthetic controller 220 is supplied by the battery of the prosthesis. Upon startup, the prosthetic controller 220 automatically connects to the prosthetic hand 110 and waits to receive signals from the implant device 20. When the prosthetic controller 220 does not receive signals from the implant device 20, it will stop sending predictions of movement to the prosthetic hand 110. This will prevent unintended or unpredictable movement of the prosthesis.

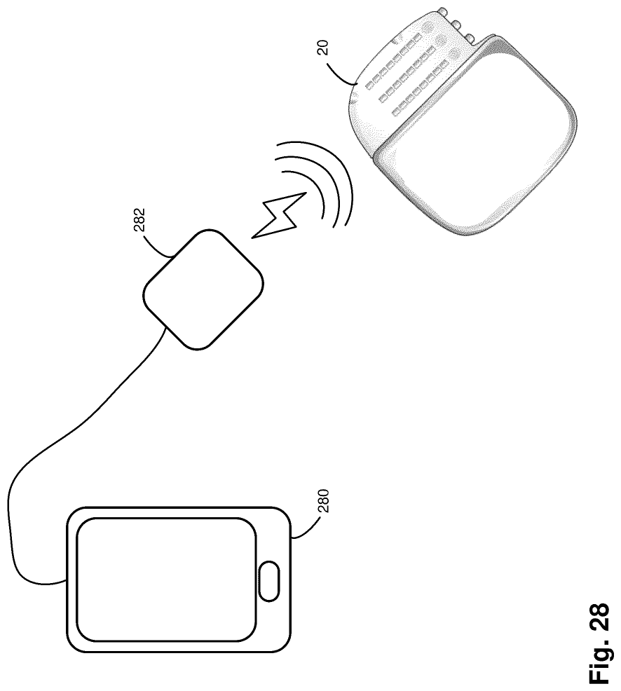

With reference to FIG. 28, an external programmer charger 280 and inductive charging pad 282 to charge the implant device 20 are shown. To charge the implant device 20 after the implant device 20 is surgically implanted in a patient, the inductive charging pad 282 can be aligned on the patient near the location of the implant device 20 within the body of the patient. For example, if the implant device 20 is implanted with a chest of the patient, as shown in FIG. 20, the inductive charging pad 282 can be placed on the patient's chest over the approximately location of the implant device 20 within the patient's chest. The external programmer charger 280 and/or the inductive charging pad 282 can generate an output, such as lighting an output light emitting diode (LED) or generating an audible sound, to indicate that the inductive charging pad 282 and the implant device 20 are aligned for inductive charging. Once aligned and communicating, the external programmer charger 280 and inductive charging pad 282 can charge the battery 230 of the implant device 20 while the implant device 20 is implanted in the patient's body. The same patient programmer charger (PPC) from the Algovita platform can be used to wirelessly recharge the internal battery of the ISU using inductive coupling at 40 kHz at 0.65 W. The PPC can also be used to check the battery level of the ISU.

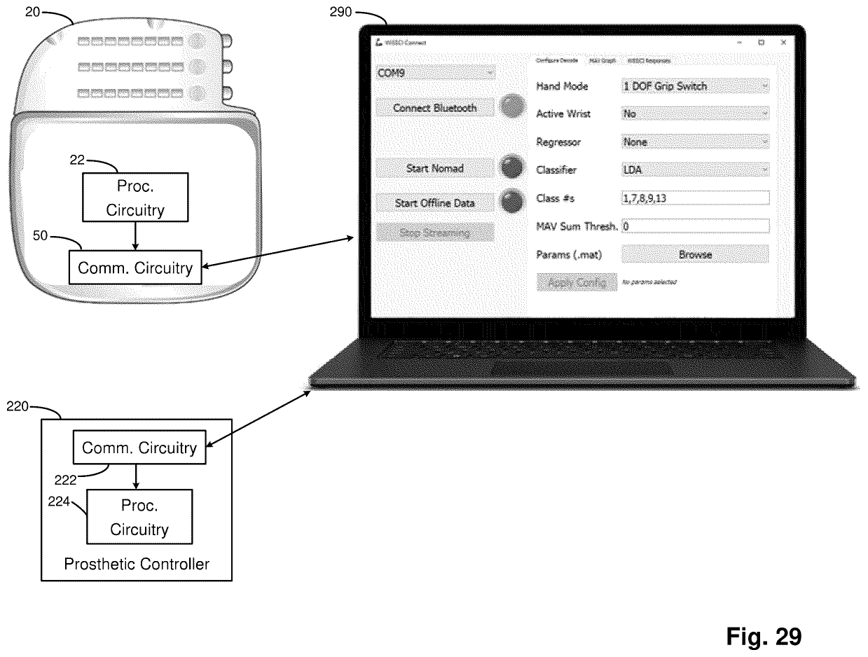

With reference to FIG. 29, an external programmer device 290 is shown in communication with the communication circuitry 50 of the implant device 20 and the communication circuitry 222 of the prosthetic controller 220. Communication between the external programmer device 290 and the communication circuitry 50 of the implant device 20 can be performed wirelessly while the implant device 20 is implanted in the patient's body. Communication between the external programmer device 290 and the communication circuitry 222 of the prosthetic controller 220 can be performed wirelessly or via a wired communication connection. The external programmer device 290 can be implemented with a computing device, such as a laptop computer, desktop computer, tablet device, mobile device, or any other suitable computing device configured with an application configured to communicate with the implant device 20 and the prosthetic controller 220. The external programmer device 290, for example, can receive the EMG signals of the amplified nerve signals 240 from the implant device 20, including either the raw EMG signals or the filtered, processed, and sampled EMG signals, as described above. For further example, the external programmer device 290 can use the filtered, processed, and sampled EMG signals from the implant device 20 to determine the configuration parameters for the prosthetic controller 220. Additionally or alternatively, the external programmer device 290 can also receive the filtered, processed, and sampled EMG signals from the communication circuitry 222 of the prosthetic controller 220 after the filtered, processed, and sampled EMG signals are received from the implant device 20. The external programmer device 290 can then determine configuration parameters to be used by the prosthetic controller 220 to control the prosthetic hand 110 based on the filtered, processed, and sampled EMG signals from the implant device 20. The external programmer device 290 and/or the prosthetic controller 220 can also use machine learning algorithms to determine and adjust the configuration parameters used by the prosthetic controller 220 to control the prosthetic hand 110 based on the filtered, processed, and sampled EMG signals from the implant device 20.

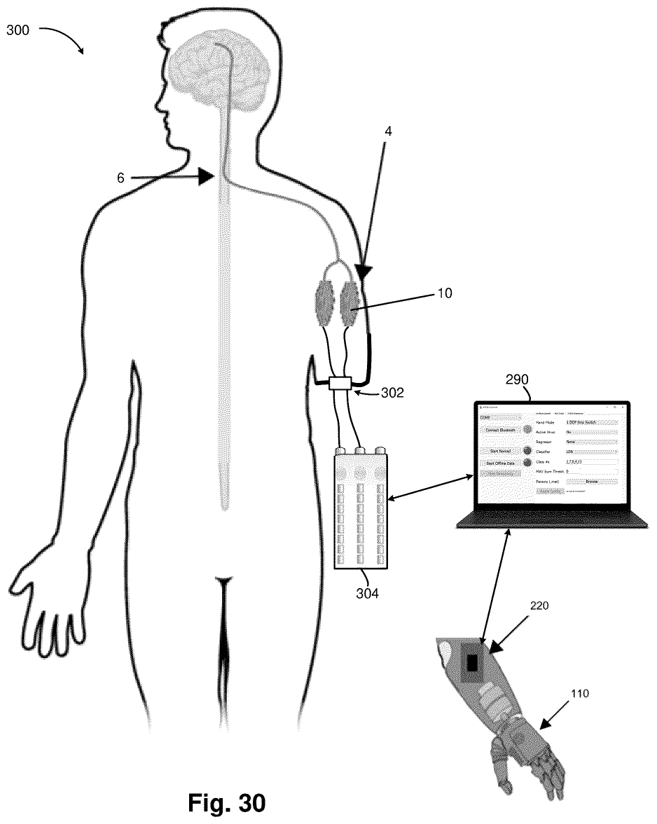

With reference to FIG. 30, another embodiment of the present disclosure is shown. The system 300 of FIG. 30 is similar to system 200 described above, except that the system 300 does not include an implant device 20 implanted within the patient. Instead, the system 300 of FIG. 30 is configured for testing and configuration prior to an implant device 20 being implanted within the patient. Similar to the system 200 described above, system 300 includes a neural interface system 4 with nerve signals from a nerve 6, such as a peripheral nerve, that have been amplified by free tissue grafts 10, as described above. In the system 300 of FIG. 30, however, the electrical leads 18 extend to outside of the body of the patient through a port 302 and connect to an interface 304. The interface 304 is connected to an external programmer device 290 that is in communication with a prosthetic controller 220 of a prosthetic hand 110. The system 300 can be used to test and calibrate the parameters of the prosthetic controller 220 prior to an implant device 20 being implanted within the body of the patient and with or without the prosthetic hand 110 being attached to the patient. In this way, the system 300 can be used to review and analyze the nerve signals generated by the nerve 6 and amplified by the free tissue grafts 10 to determine whether the signals would be sufficient for use with a prosthetic controller 220 to control a prosthetic hand 110 prior to the implant device being implanted into the patient.

As described above, the implant device 20 of the present disclosure can be used both for generating prosthetic control signal outputs 56 that are used to control a prosthetic device, such as prosthetic hand 110, and for receiving stimulation feedback signals, such as prosthetic sensor input signals 58 generated by one or more pressure sensors 225, that are used to provide stimulation and sensory feedback signals to the nerve 6. For example, the implant device 20 can include one or more Intan RHS2116 stimulator/amplifier chips configured to stimulate neural tissue and sense biopotentials. The RHS2116 stimulator/amplifier chip provides current controlled stimulation pulses to individual electrode contacts up to 2.55 milliamps (mA), which has been shown to be sufficient to elicit sensor percepts. Stimulation pulses can be delivered in a biphasic manner for charge balancing. The RHS2116 stimulator/amplifier chip has a fast amplifier reset feature to eliminate stimulation artifacts prior to sensing biopotentials. The RHS2116 stimulator/amplifier chip can be powered by a 3.3 volt linear voltage regulator, such as the TPS7A2033 by Texas Instruments. Additionally or alternatively, a dual polarity output power supply can be used to provide positive and negative voltage supplies for stimulation to the RHS2116 stimulator/amplifier chip up to plus or minus seven volts (+/−7 V).

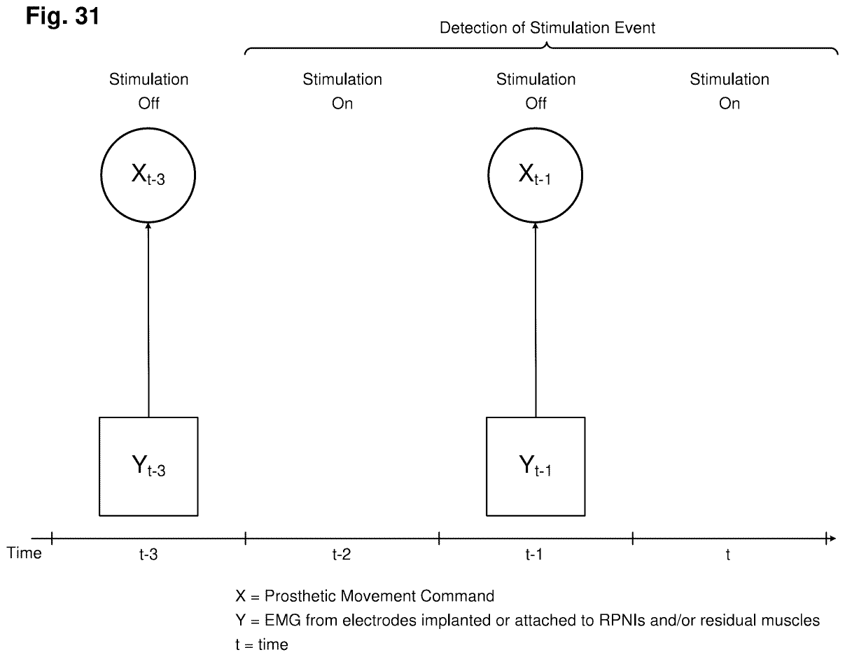

In one embodiment, the systems and methods of the present disclosure can be used for both (1) receiving signals from the free tissue graft for generating prosthetic control signal outputs 56 that are used to control a prosthetic device, such as prosthetic hand 110, and (2) receiving stimulation and sensory feedback signals, such as prosthetic sensor input signals 58 generated by one or more pressure sensors 225 of the prosthetic device, such as the prosthetic hand 110. One issue, however, with performing simultaneous motor control functionality and sensory perception/stimulation functionality at the same time is that the stimulation signals can create artifacts or noise in the signals being sensed and recorded for motor control of the prosthetic device. As such, the implant device 20 of the present disclosure is configured to mitigate and/or eliminate the signal corruption and artifact issue using one or more of the approaches and algorithms described below with reference to FIGS. 31-33, which illustrate different approaches and algorithms to mitigate and/or eliminate signal corruption and artifacts in the signals being sensed and recorded for motor control of the prosthetic device.

For example, with reference to FIG. 31, an algorithm for generating prosthetic movement commands and performing stimulation in alternating time periods is illustrated. In FIGS. 31-33, time periods t-3, t-2, t-1, and t are illustrated along the horizontal axis from left to right. The time periods, for example, can be 10 to 50 millisecond time periods, although any suitable time period length can be used. Further, X represents prosthetic movement commands generated by the implant device 20 during the time period indicated in subscript. In addition, Y represents the EMG signals received from the electrodes implanted or attached to RPNIs, residual muscles, and/or target muscles after a TMR procedure during the time period indicated in subscript. At time period t-3, the stimulation functionality is initially turned off and the implant device 20 generates a prosthetic movement command Xₜ₋₃ based on EMG signals Yₜ₋₃. At time period t-2, a stimulation event is detected that lasts throughout time periods t-2, t-1, and 5 of the examples. The stimulation event, for example, can be receiving signals from a pressure sensor 225 of the prosthetic device. In other words, stimulation can be initiated and used once a need to provide stimulation for sensor perception is detected, such as when a pressure sensor 225 detects an increase in pressure to the prosthetic device and generates a pressure signal.

In the approach and algorithm illustrated in FIG. 31, the implant device 20 alternates between performing stimulations for sensory perception and generating prosthetic movement commands during consecutive time periods. For example, stimulation for sensory perception is performed when stimulation functionality is on during time periods t-2 and t and stimulation functionality is off and a prosthetic movement commands Xₜ₋₂ and Xₜ₋₁ are generated during time periods t-3 and t-1 based on EMG signals Yₜ₋₃ and Yₜ₋₁. In this way, prosthetic movement commands are generated during time periods t-3 and t-1 and stimulation for sensory perception is performed during time periods t-2 and t. In this way, the generation of prosthetic movement commands is temporarily paused during time periods t-2 and t while stimulation is being performed. Similarly, stimulation is temporarily paused during time periods t-3 and t-1 while prosthetic movement commands are being generated. In the example of FIG. 31, after applying stimulation and then moving to the next time period for generating a prosthetic movement command, such as t-1, the position of the prosthetic and/or the state of the prosthetic command is at the same position or state as it was at the end of the previous time period for generating prosthetic movement commands, e.g., t-3. In other words, in the approach and algorithm of FIG. 31, the implant device 20 alternates time windows between (A) generating prosthetic movement commands X with stimulation off and (B) pausing prosthetic control off with stimulation on. In this way, the implant device 20 switches back and forth between receiving stimulation and sending control signals every time period, such as every 10 to 50 milliseconds.

In the approach and algorithm of FIG. 31, the prosthetic movement commands are not updated during the stimulation time periods, e.g., t-2 and t. In other words, if the prosthetic movement command at the end of time period t-3 commands the prosthetic hand 110 to close at a speed indicated by the command Xₜ₋₃, then at the beginning of time period t-1, the prosthetic hand will still be closing at that same speed indicated by the previous command Xₜ₋₃. This approach and algorithm can be implemented with a number of control algorithms, including regressors that estimate kinematics, such as linear regression or neural networks, or classifiers that estimate discrete states, such as linear discriminant analysis, Naive Bayes, support vector machines, and/or neural networks.

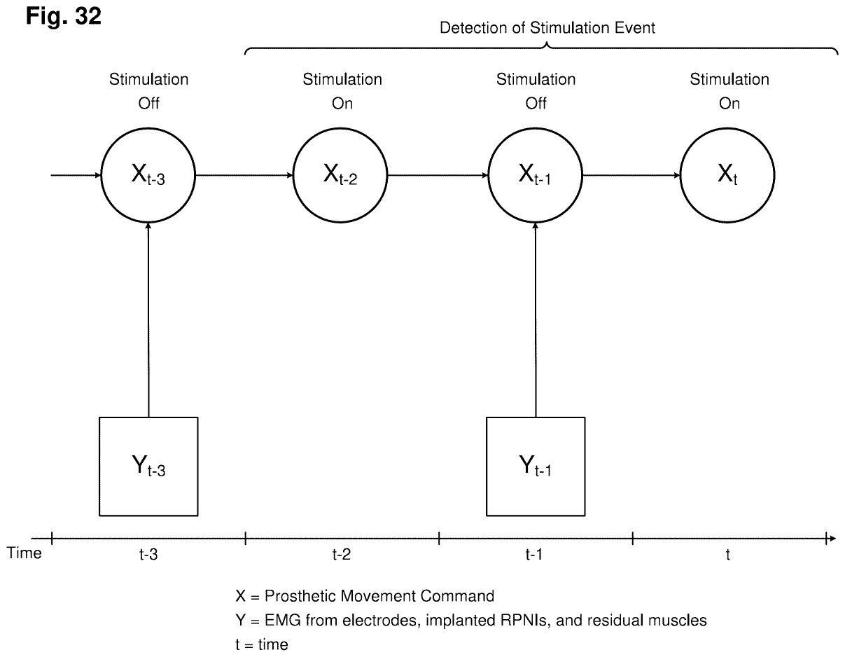

With reference to FIG. 32, another algorithm for generating prosthetic movement commands and performing stimulation is illustrated. The approach and algorithm illustrated in FIG. 32 is similar to the approach and algorithm illustrated with reference to FIG. 31, except that the approach and algorithm illustrated in FIG. 32 estimates a prosthetic movement command during the time periods when stimulation is being performed. For example, in time periods t-2 and t, the implant device 20 generates estimated prosthetic movement commands Xₜ₋₂ and Xₜ based on previously generated prosthetic movement commands, such as Xₜ₋₃ and Xₜ₋₁. For example, instead of simply pausing the generation of prosthetic movement commands during the stimulation time periods, as is done by the approach and algorithm of FIG. 31, the approach and algorithm of FIG. 32 instead provides an estimated prosthetic movement command, such as in time periods t-2 and t, to provide a smoother transition for the generation of prosthetic commands in time periods t-3 and t-1. This approach and algorithm can utilize a recursive or regression algorithm that estimates kinemetics, such as a Kalman filter or particle filter, or a classifier that estimates discrete states, such as a Markov model. In this way, the implant device 20 estimates movement states recursively over time using both incoming signal measurements and a mathematical process model. Similar to the approach of FIG. 31, the approach of FIG. 32 alternates between sensing and receiving signals from the electrodes, such as in time periods t-3 and t-1, and sending stimulation signals for sensory perception, such as in time periods 5-2 and 5. Further, the implant device 20 does not read or sense any signals from the electrodes while performing stimulation during, for example, time periods t-2 and 5. The implant device 20, however, can generate an estimated prosthetic movement command, e.g., Xₜ₋₂ and Xₜ, to provide a smoother series of commands over time instead of pausing the generation of prosthetic movements commands during the time periods of performing stimulation. In this way, the approach and algorithm of FIG. 32 can blend between two consecutively generated commands, e.g., Xₜ₋₃ and Xₜ₋₁, and avoid jumps between commands that may occur in the approach and algorithm of FIG. 31 when the generation of commands is paused. In this way, the approach and algorithm of FIG. 32 can accommodate intermittent measurements and provide smooth command updates during time periods where stimulation for sensory perception is performed.

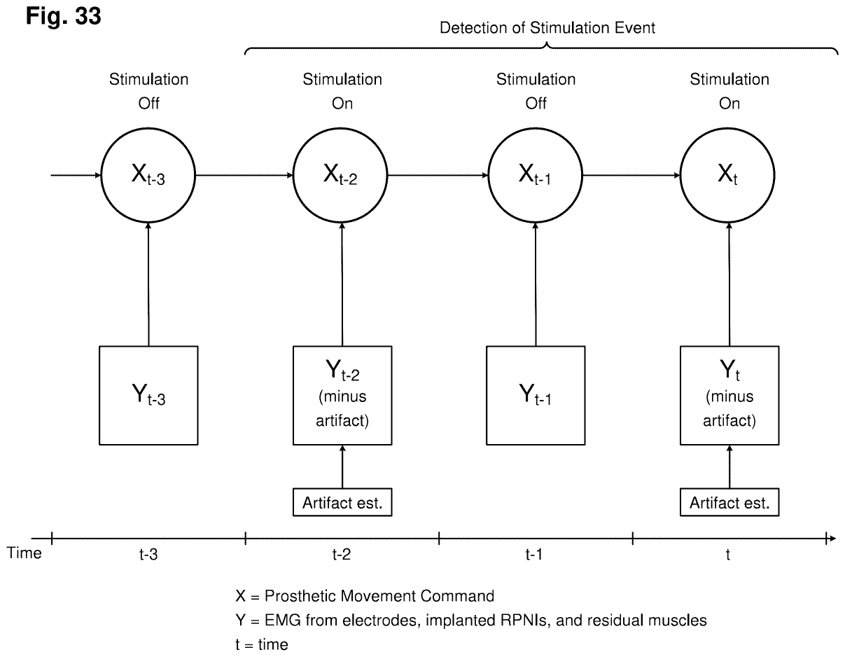

With reference to FIG. 33, another algorithm for generating prosthetic movement commands and performing stimulation is illustrated. In the approach and algorithm of FIG. 33, prosthetic movement commands are generated in all time periods. However, during time periods when stimulation is also performed, e.g., t-2 and t, the implant device 20 performs artifact estimation to estimate artifacts that have been introduced into the EMG signals Y from the electrodes due to the stimulation, and then subtracts or filters out the estimated artifacts from the EMG signals Y and generates a prosthetic movement command X based on the resulting/filtered EMG signals resulting after artifact subtraction. In this way, the implant device 20 can remove the artifacts that may have been introduced into the EMG signals as a result of performing stimulation while simultaneously reading nerve signals from the electrodes attached to the free tissue grafts 10. Put another way, the implant device performs noise reduction, filtering, or cancelation on the EMG signals Y to remove artifacts that may have been introduced to the signals by performing stimulation for sensory perception.

For example, a template subtraction algorithm can be used to generate an expected artifact based on a given stimulation signal or set of stimulation signals. The template can be used to subtract the artifact from the EMG signals. In other words, under this approach, the EMG signals Y may become corrupted due to the stimulation for sensory perception, but the algorithm or approach filters the EMG signals Y to subtract the artifacts so that prosthetic movement commands can be generated based on the filtered EMG signals Y. For example, the template subtraction algorithm can be implemented by averaging artifacts immediately following stimulation pulses using multiple exponential filters with a filter learning rate. In this way, aa representative template can be constructed which is then subtracted from the original signal to yield an estimated artifact-free signal.

Additionally or alternatively, an E-Normalized Least Mean Squares algorithm can be used to remove the unwanted artifact from the EMG signals Y. For example, the E-Normalized Least Mean Squares algorithm can be implemented as an improved version of a standard Least Means Squares algorithm, yielding better performance for signals with intervals of larger and lower signal energy. In the &-Normalized Least Mean Squares algorithm, an adaptive filter relies on a reference signal highly correlated with the stimulation artifact. The algorithm can adapt to varying artifact waveforms without requiring completely relearning of the weights used by the algorithm. For example, the &-Normalized Least Mean Squares adaptive filter algorithm convolutes the reference signal with filter weights to predict an artifact waveform. The predicted artifact is subtracted from the original signal to yield an estimated artifact-free signal, which is then also used to update the filter weights after each sample.

While the example of FIG. 33, illustrates stimulation being performed in alternating time periods, stimulation could also be performed continuously in consecutive time periods while reading the EMG signals Y and filtering/subtracting any artifacts introduced into the EMG signals Y. However, stimulation could be performed in alternating time periods, as shown in the example of FIG. 33, to conserve battery power of the implant device 20.

While any single algorithm or approach of FIGS. 31-33 could be used, the implant device 20 can alternatively be configured to perform all three approaches and/or switch between approaches to determine and select the best approach or algorithm for a particular environment or scenario.

While FIGS. 31-33 illustrate approaches wherein stimulation for sensory perception is performed in alternating time periods, another sequence could alternatively be used. For example, stimulation could be performed in two or more consecutive time periods followed by one or more time periods of reading EMG signals Y for generating prosthetic movement commands.

### Multiple Sensing Electrodes

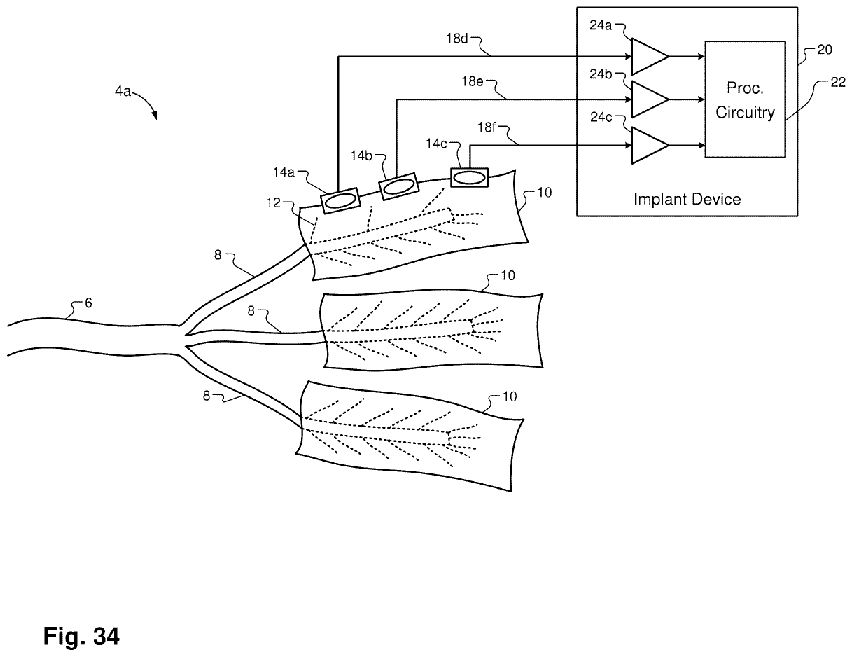

With reference to FIG. 34, another embodiment of the present disclosure is shown. In particular, a neural interface system 4a is shown, which is similar to the neural interface system discussed above with reference to FIG. 1 and includes free tissue grafts 10 and an implant device 20 with processing circuitry 22. The embodiment of FIG. 34, however, includes multiple sensing electrodes 14a, 14b, 14c on a single free tissue graft 10. The sensing electrodes 14a, 14b, 14c sense and receive nerve signals amplified by the free tissue graft 10 at different locations on the free tissue graft 10. The sensed and received signals are communicated from the sensing electrodes 14a, 14b, 14c to the implant device 20 via wires 18d, 18e, 18f. The wires 18d, 18e, 18f can be configured in a single wire bundle with the individual wire ends being separately connected to the electrodes 14a, 14b, 14c and the implant device 20. The sensed and received signals can be amplified by amplifiers 24a, 24b, 24c and transmitted to the processing circuitry 22. In this way, the multiple electrodes 14a, 14b, 14c can be located at multiple different sites on a single free tissue graft 10 of an RPNI. As discussed above, one or more portions (nerve fascicles 8) of the nerve 6 can regenerate within the free tissue graft 10 reinnervating the tissue. The reinnervation can include growing sprout nerve fibers 12, which can reinnervate across the entire muscle tissue. The multiple electrodes 14a, 14b, 14c can be used at different locations on the free tissue graft 10 to sense and receive different motor control signals from the RPNI. The different signals can be received by the implant device 20 as different motor control inputs and used for different purposes, such as controlling different parts of a prosthetic. For example, the multiple electrodes 14a, 14b, 14c at different locations on the single free tissue graft 10 of an RPNI can be used to receive different motor control signals that are, in turn, used to control different fingers of a prosthetic hand 110. While a prosthetic hand 110 is provided as an example, any other type of prosthetic device can be used with the systems and methods of the present disclosure.

In this way, a multi-electrode array, e.g., electrodes 14a, 14b, 14c, can sense independent motor signals from a single RPNI construct for high fidelity prosthetic control. When a nerve with multiple functions innervates a free tissue graft 10, functionally distinct motor end plates are innervated throughout the area of the free tissue graft 10. As discussed above with reference to FIG. 1, a single electrode can be used on each free tissue graft 10 to sense and record the summation of nearby motor activity. In cases where single or limited functionality is required from a nerve, this approach may be sufficient. The use of a single electrode, however, can provide a less complete and lower resolution reading of the signals from the nerve and the nerve's capabilities, as compared with the neural interface system 4a of FIG. 34, which includes multiple sensing electrodes 14a, 14b, 14c on a single free tissue graft 10. The present embodiment can include an array of electrodes 14a, 14b, 14c with a single wire bundle, i.e., bundled wires 18d, 18e, 18f, and multiple electrode contacts, e.g., high density electrode devices that include many contacts, or a system in which multiple leads and electrodes are independently implanted into the RPNI. This device allows independent signals to be recorded from a single RPNI free tissue graft 10. For example, in transhumeral amputations the severed nerves are responsible for multiple finger and thumb functions. Even if a nerve can be divided to create a few RPNIs, it is highly likely the RPNIs will each contain multiple functions. The multi-channel electrodes of the present embodiment can record independent signals from the RPNI and greatly improve the fidelity of prosthetic control.

### Multiple Sensing Electrodes for Regenerative Peripheral Nerve Interface (RPNI)

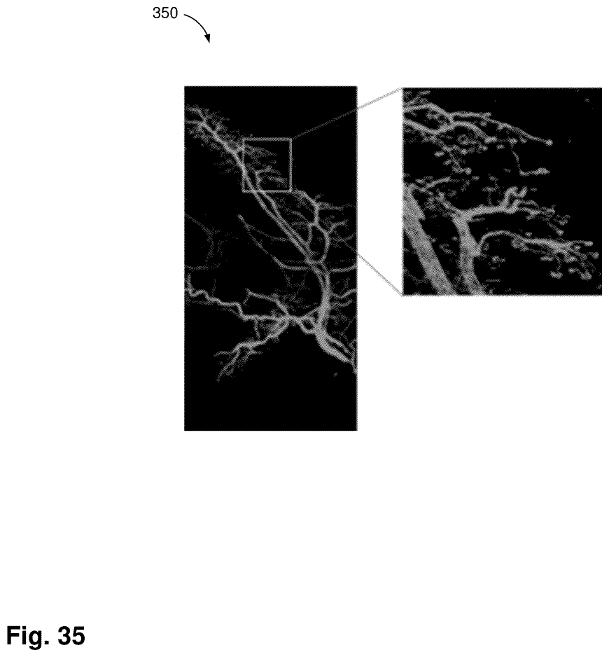

The basic functional unit of the skeletal muscle is the motor unit, which refers to a motor neuron and all the myofibers it innervates. Individual muscles contain hundreds of motor units, each with varying numbers of fibers that produce different levels of contraction force. In an RPNI, motor units are formed as motor neurons in a transected nerve reinnervate existing fibers in the muscle tissue graft. As illustrated in FIG. 35, histological imaging 350 showing different motor units forming neuromuscular junctions within a single RPNI.

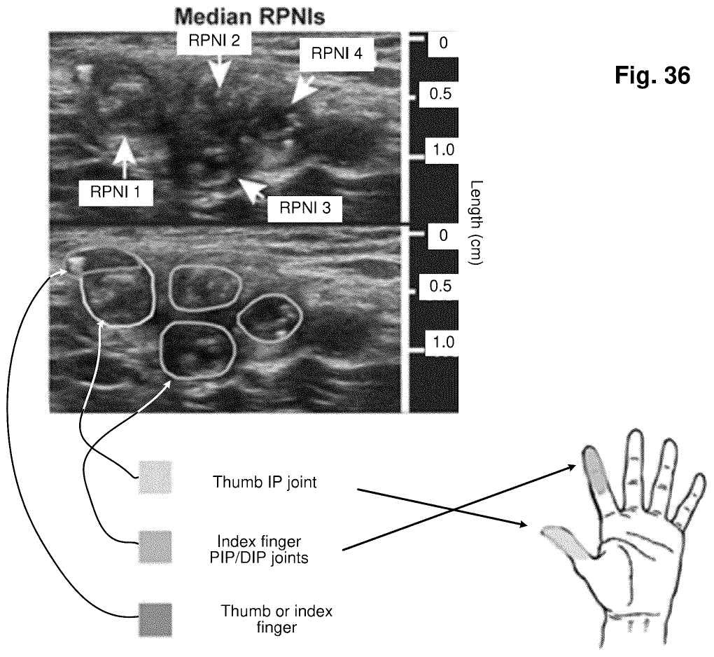

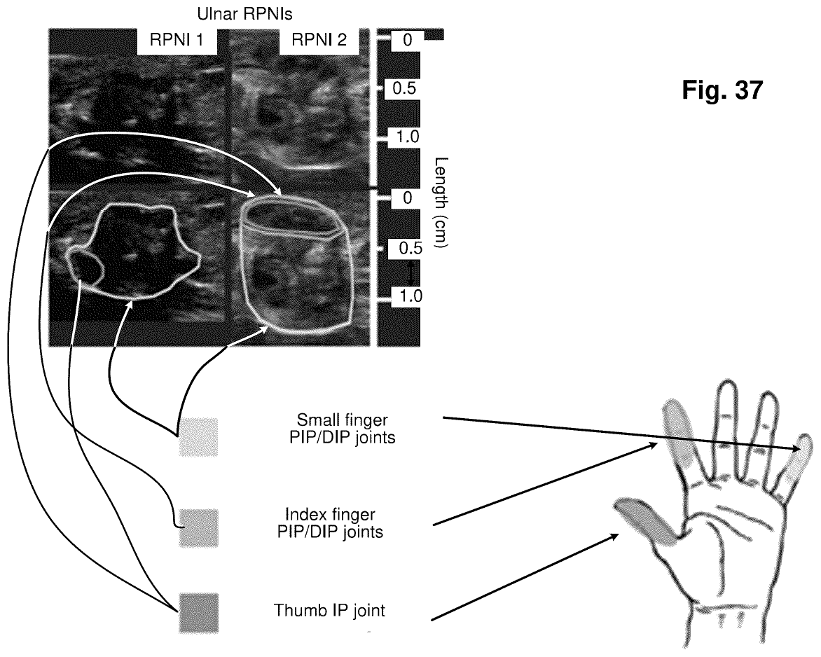

A singular RPNI can be created on a branch of a larger nerve or, alternatively, larger nerves can be split into several groups of fascicles, with a different RPNI construct for each fascicle group. The hand and fingers are controlled by thirty-four muscles. Thumb movement itself is controlled by nine muscles, half of which are within the hand. For example, if the median nerve is split to form two-to-three RPNIS, each RPNI will have reinnervation territories from fascicles that have multiple functions. In other words, a median RPNI may be reinnervated by fascicles that control thumb opposition as well as fascicles that control index finger flexion. Motor units in an RPNI may therefore independently activate for multiple distinct movements corresponding to the somatotopy of the nerve. When an RPNI is created on a nerve that controls multiple hand functions, the muscle tissue can be reinnervated with a motor unit topography that produces distinct contraction regions for different movements. FIG. 36 illustrates different regions of RPNIs on a median nerve that contract in response to different individual finger and thumb movements when viewed under ultrasound. Similarly, FIG. 37 illustrates different regions of RPNIs on an ulnar nerve that contract in response to different individual finger and thumb movements when viewed under ultrasound. In FIGS. 36 and 37, the arrows illustrate which areas of the RPNIs in the ultrasound image map to which joints of the fingers in the hand.

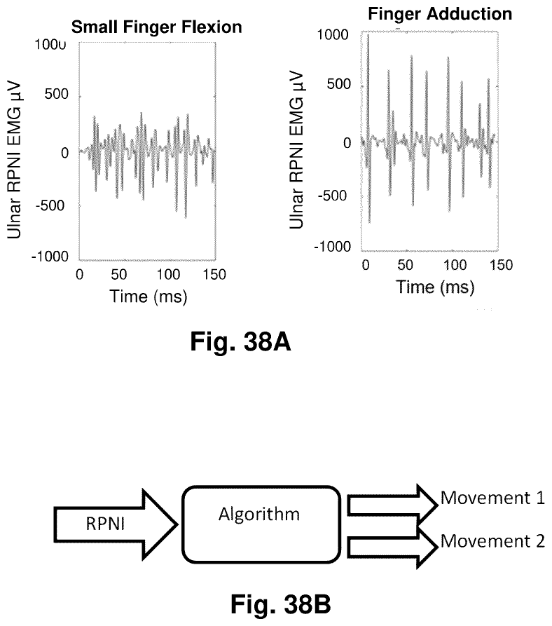

Within an RPNI, individual motor unit activity can be recorded by a single implanted electrode. In the example illustrated in FIGS. 38A and 38B, distinct motor units activate for different movements, e.g., small finger flexion and finger adduction, controlled by the ulnar nerve. Existing control strategies rely on software to parse out these two movements different movements from a single RPNI.

Electromyography from a single electrode is either processed into features and an algorithm interprets differences in the processed signals, or individual motor units are inferred via signal decomposition. However, these methods cannot reliably scale as the complexity and number of movements increases relative to the number of RPNIs. Furthermore, there is an exponential decay in signal amplitude with distance from the electrode recording surface. Simulations have estimated that the majority of motor unit activity recorded from intramuscular electrodes may come from muscle fibers within 1.5 mm to 2 mm of the electrode, depending on the size and contact spacing. Motor units further away from the electrode are recorded with a lower signal-to-noise ratio and do not as reliably captured as nearby motor units.

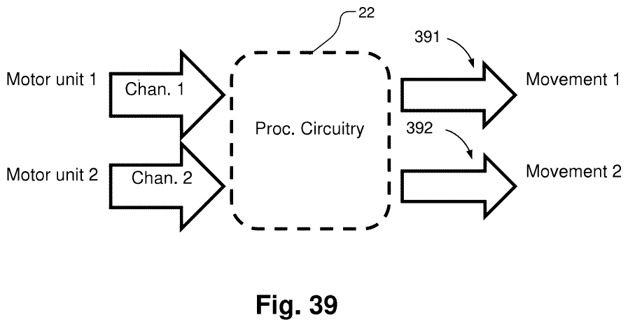

With the systems and methods of the present disclosure, multiple sensing electrodes can capture activity from different motor units on independent hardware channels. For example, FIG. 39 illustrates two motor units 1 and 2 connected to two channels 1 and 2, respectively, that are input to processing circuitry 22 for processing. This enables control of a larger number of movements with increased dexterity. Importantly, this is a fundamentally more reliable method to increase signal resolution than software decomposition. The recorded signal from the individual contacts of the multiple sensing electrodes will be dominated by the closest motor units. Continuing the two-movement example from above, motor unit 1 can be directly mapped to movement 1, and motor unit 2 to movement 2. For example, the processing circuitry 22 can utilize the input received via channel 1 to generate control signal 391 that corresponds movement 1 and can utilize the input received via channel 2 to generate control signal 392 that corresponds to movement 2. This mapping may be performed manually, or an algorithm may be used to learn the relationship out of convenience. The increase in signal independence guarantees that this method will perform more reliably than previous methods that use a single channel and software decomposition.

Implanting multiple sensing electrodes can also increase the sampled area to fully cover the RPNI. Due to both of these factors, multiple sensing electrodes can more fully utilize the inherent information in a single RPNI construct for prosthetic control.

In one embodiment, instead of implanting a single electrode into an RPNI, multiple instances of the same electrode can be implanted in opposing areas or lengths of the RPNI. For example, FIG. 34 illustrates sensing electrodes 14a, 14b, 14c along the length of a single free tissue graft 10. These electrodes may have a similar geometry to currently used intramuscular EMG electrodes. Each electrode can have either a single monopolar recording contact or two bipolar (differential) contacts and is typical implanted along the length of the RPNI. In one example, to optimize for placement of multiple electrodes in an RPNI, each electrode can have a diameter less than 1.5 mm, a recording contact length of 2mm-1cm, with a bipolar inter-contact spacing less than 2 cm. While these dimensions and positioning are provided as examples, other dimensions and positioning can also be used. Implanting two or more of these electrodes in this manner can sufficiently span a larger RPNI and record independent signals from different functional regions.

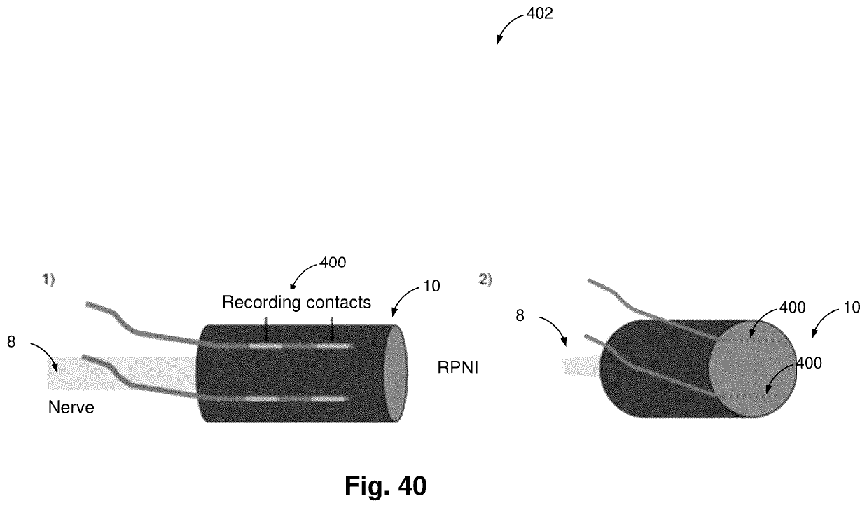

In another embodiment, as shown in FIG. 40, recording resolution can be further improved with an increased number of smaller recording contacts 400 for each electrode. For example, a high-resolution electrode can have two or more contacts that are smaller, e.g., less than 2 mm. An example electrode of this variety can have eight contacts that are each 0.2 mm or less. While these dimensions are provided as an example, other dimensions can also be used. Each contact of the electrode selectively records a few distinguishable and independent motor units. A high-resolution electrode may be implanted through the width of the RPNI to traverse multiple muscle fibers, as shown in FIG. 40, along the length of the RPNI. Alternatively, a matrix array of contacts that spans multiple dimensions of the RPNI can be used. Single or multiple high-resolution electrodes may be inserted into the RPNI.

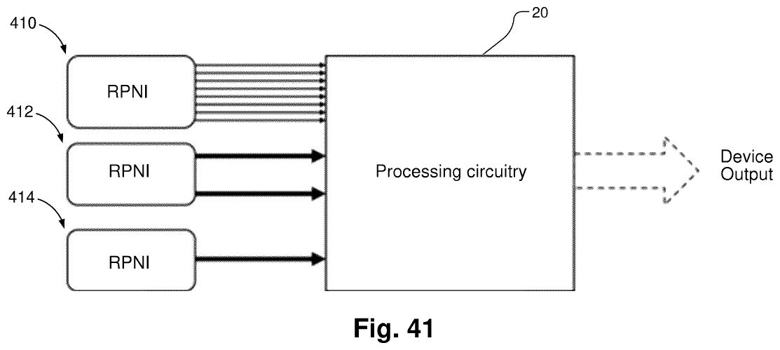

With reference to FIG. 41, the processing circuitry 20 can include multiple independent input channels for each RPNI. For example, RPNI 410 generates output for eight channels that are input to the processing circuitry 20. RPNI 412 generates output for two channels that are input to the processing circuitry. RPMI 414 generates output for one channel that is input to the processing circuitry 20. In the example of FIG. 41, the processing circuitry can be included in a single implant device 20, as described above, with amplifiers and circuitry to receive the input from the various channels from the RPNIs 410, 412, 414.

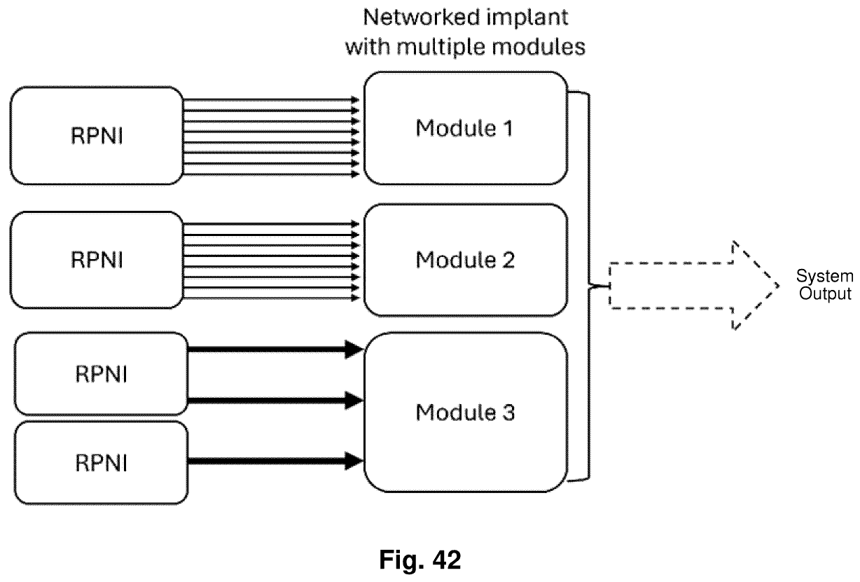

With reference to FIG. 42, in another embodiment, if multiple high-resolution electrodes are used, a network of implant devices with multiple modules can be used. For example, a wireless body area network (BAN) can be used for communication by and between the multiple separate implant devices/modules.

Alternatively, a wired network can be used to connect and enable communication by and between the multiple separate implant devices/modules. Each module can include the hardware, circuitry, memory, and processors described above as being included in the processing circuitry 22. In addition, the modules can include sufficient communication hardware and software stored in memory to enable communication between the modules. In this way, the modules can receive input from the various input channels of the RPNIs and coordinate to generate a system output to control a prosthetic device. FIG. 42, for example, shows three modules Module 1, Module 2, and Module 3. Modules 1 and 2 are each connected to eight channels that receive input generated by corresponding associated RPNIs. Module 3 is connected to a first RPNI that generates input for Module 3 on two channels and to a second RPNI that generates input for Module 3 on a single channel.

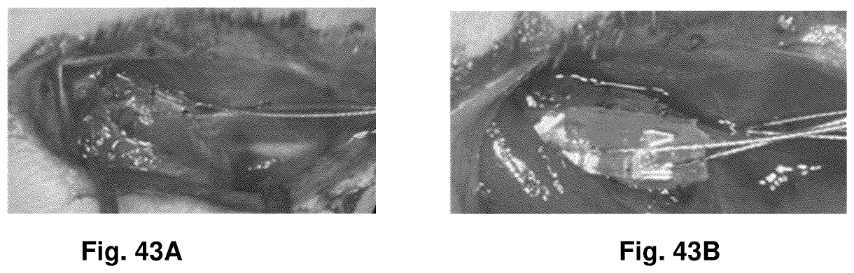

FIGS. 43A and 43B are photographs of RPNIs with multiple sensing electrodes. FIG. 43A shows an RPNI having a patch electrode on the back of the RPNI. FIG. 43B shows the RPNI having a patch electrode on the front of the RPNI as well as a patch electrode on the back of the RPNI.

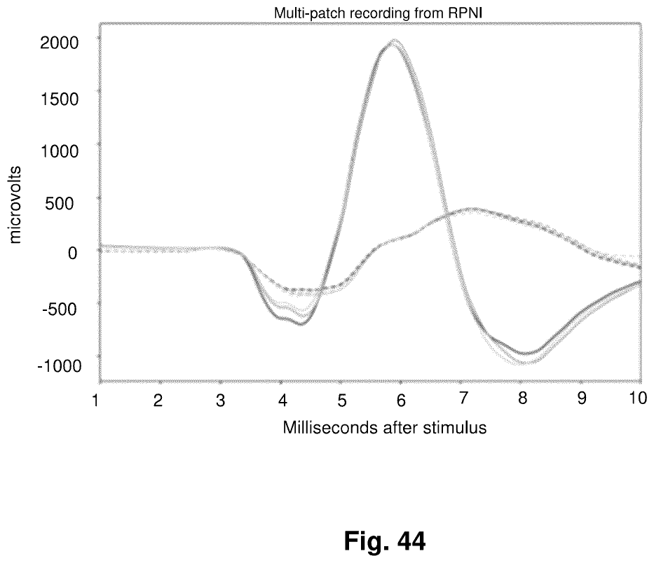

With reference to FIG. 44, two bipolar patch electrodes were implanted on the same RPNI, equidistant to the stimulating nerve cuff. In FIG. 44, patch 1 corresponds to the solid graph lines and patch 2 corresponds to the dashed graph lines, which show the recordings obtained from the two patch electrodes in the same recording window. Stimulation of the nerve cuff generated independent motor signaling with different waveforms and max amplitudes, demonstrating that recording selective signals on independent hardware channels would be needed to capture the different signals spanning the entire RPNI.

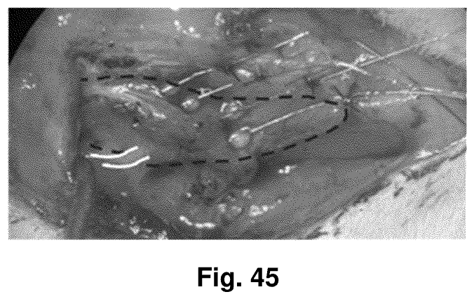

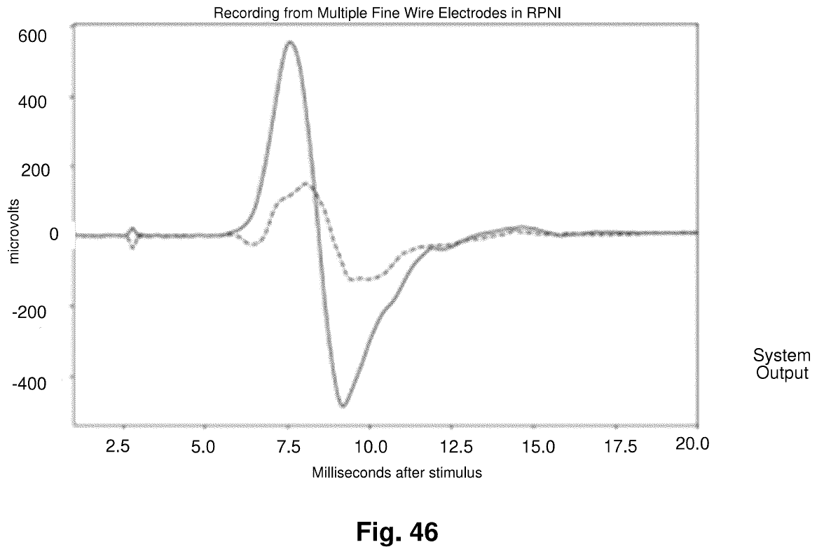

With reference to FIGS. 45 and 46, two sets of fine wire electrodes were implanted on the same RPNI equidistant to the stimulating nerve cuff. In FIG. 45, the dashed line illustrates the location of the RPNI. The sets of fine wire electrodes can be seen attached to the RPNI. With reference to FIG. 46, similar to the patch electrode findings, different waveforms were recorded from the two sets of electrodes. In the graph of FIG. 46, one fine wire set of electrodes corresponds to the solid line and a second fine wire set of electrodes corresponds to the dashed line. Similar to an RPNI, the various constructs described further below have independent motor units in the muscle tissue. While some of the embodiments below include single electrodes in the muscle and single electrodes in the skin, the embodiments can be similarly expanded to include multiple sensing electrodes in place of each single electrode to capture more independent signals.

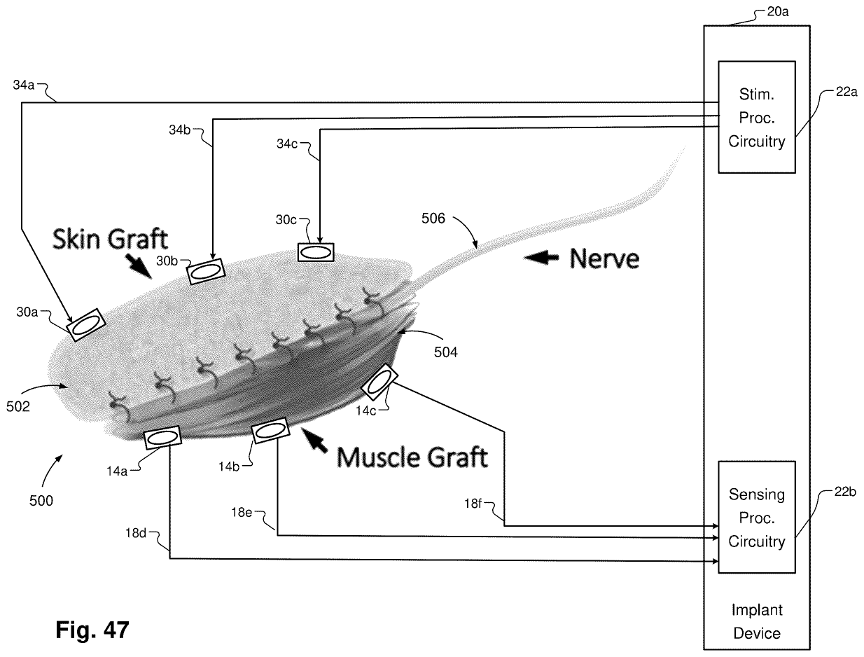

Composite Regenerative Peripheral Nerve Interface (C-RPNI) With reference to FIG. 47, another embodiment is shown that includes utilizing a composite regenerative peripheral nerve interface (C-RPNI) construct 500 that includes a combination of a free skin or dermal graft 502 as well as a muscle graft 504 secured around a target mixed sensorimotor nerve 506 that is implanted between the dermal graft 502 and the muscle graft 504. Once implanted, the nerve 506 demonstrates preferential targeted reinnervation such that sensory nerve fibers reinnervate the dermal graft 502 and motor nerve fibers reinnervate the muscle graft 504. In this way, the dermal graft is reinnervated with sensory feedback nerve fibers connected to the sensorimotor nerve 506 and motor control nerve fibers also connected to the sensorimotor nerve 506 reinnervate the muscle graft 504.

As shown in FIG. 47, stimulation electrodes 30a, 30b, 30c are attached to the dermal graft 502 and provide stimulation signals, transmitted via wires 34a, 34b, 34c from an implant device 20a, to the sensory nerve fibers within the dermal graft 502. Similarly, sensing electrodes 14a, 14b, 14c are attached to the muscle graft 504 and sense motor control signals from the motor control nerve fibers within the muscle graft 504. The sensing electrodes 14a, 14b, 14c transmit the sensed motor control signals to the implant device 20a via wires 18d, 18e, 18f. While the embodiment of FIG. 47 illustrates three stimulation electrodes 30a, 30b, 30c and three sensing electrodes 14a, 14b, 14c, any number of sensing or stimulation electrodes can be used in accordance with the present disclosure. For example, in accordance with the present disclosure, a construct having one sensing electrode and one stimulation electrode can be used. In addition, in accordance with the present disclosure, a construct having multiple sensing electrodes and only one stimulation electrode can be used. In addition, in accordance with the present disclosure, a construct having one sensing electrode and multiple stimulation electrodes can be used. In addition, in accordance with the present disclosure, a construct having more than one, e.g., two, three, four, etc., sensing electrodes, and more than one e.g., two, three, four, etc., stimulation electrodes can be used.

The implant device 20a is similar to the implant devices 20 discussed above, except that implant device 20a includes separate processing circuitry for stimulation and for sensing. For example, the implant device 20a includes stimulation processing circuitry 22a connected to wires 34a, 34b, 34c to transmit stimulation signals from the implant device 20a to stimulation electrodes 30a, 30b, 30c. The implant device 20a also includes sensing processing circuitry 22b connected to wires 18d, 18e, 18f to receive sensed motor control signals from electrodes 14a, 14b, 14c.

The motor control signals received by electrodes 14a, 14b, 14c can be used by the sensing processing circuitry 22b and implant device 20a to perform motor control of a prosthetic device. Similarly, the implant device 20a and stimulation processing circuitry 22a can receive sensory feedback information from the prosthetic device and generate and transmit stimulation signals transmitted to electrodes 30a, 30b, 30c to provide sensory feedback signals to the sensorimotor nerve 506. In this way, the C-RPNI construct 500 can simultaneously amplify motor signals via muscle graft 504 while also providing sensory feedback via the dermal graft 502.

In accordance with the present disclosure, a device with multiple sensing electrodes can be used with a multi-tissue RPNI graft, i.e., a C-RPNI having both dermis and muscle tissue. For example, a single electrode implanted in the muscle and a single electrode implanted in the skin can be used independently to provide simultaneous or real-time bi-directional interfacing. A multi-channel electrode device can also be used to further enhance the selectivity of this interface, multiple sensing electrodes may record independent signals from the muscle, while a multi-contact stimulation electrode (discussed above) may be used to activate different fibers or perform current steering on the dermal tissue for selective sensory feedback.

### Muscle Cuff Regenerative Peripheral Nerve Interface (MC-RPNI)

In addition, while the above embodiments generally describe a nerve end that is placed within an RPNI or C-RPNI to reinnervate the muscle tissue of the RPNI or C-RPNI, any of the above embodiments can also be used with an RPNI or C-RPNI that is attached to surround an intact nerve. For example, any of the above embodiments can be used a muscle cuff regenerative peripheral nerve interface (MC-RPNI) having a construct with a free muscle graft and/or a free dermal graft secured circumferentially to an intact peripheral nerve. The free muscle graft and/or free dermal graft then becomes reinnervated by the contained nerve and can amplify the nerve signals to facilitate reliable and accurate detection of motor intent and to communicate sensory feedback stimulation to the intact nerve. MC-RPNI devices can be used with powered exoskeleton devices to restore function to those with extremity weakness. In this way, an RPNI or C-RPNI construct can be circumferentially attached around an intact nerve to create an MC-RPNI so that the intact nerve reinnervates the deinnervated muscle graft. The muscle graft of the RPNI amplifies the signals, which can be sensed with electrodes and used to control, for example, an exoskeleton. In addition, the dermal graft can amplify stimulation signals to stimulate and provide sensory feedback to the intact nerve.

In accordance with the present disclosure, a device with multiple sensing electrodes can also be used to record independent signals from an MC-RPNI circumferentially attached around a portion of a nerve, instead of a nerve ending. The MC-RPNI can be a single tissue construct (e.g., having only a muscle graft or dermal graft) or multiple-tissue construct (e.g., having both a muscle graft portion and a dermal graft portion) and the device can provide selective interfacing to control an exoskeleton or other prosthetic or medical device. In a multi-tissue embodiment, the MC-RPNI construct can have a different geometry than the C-RPNI construct for a nerve ending. For example, concentric wrapping of tissues may be used in the MC-RPNI, as compared to a “sandwich” type configuration in a nerve ending embodiment. In addition, different geometries or implantation procedures can be used for multiple electrodes, such as multiple sensing and/or multiple stimulation electrodes.

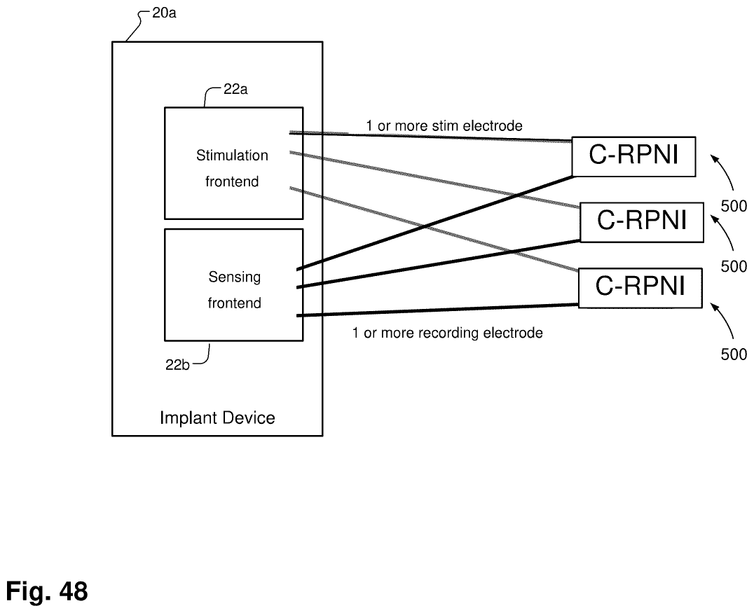

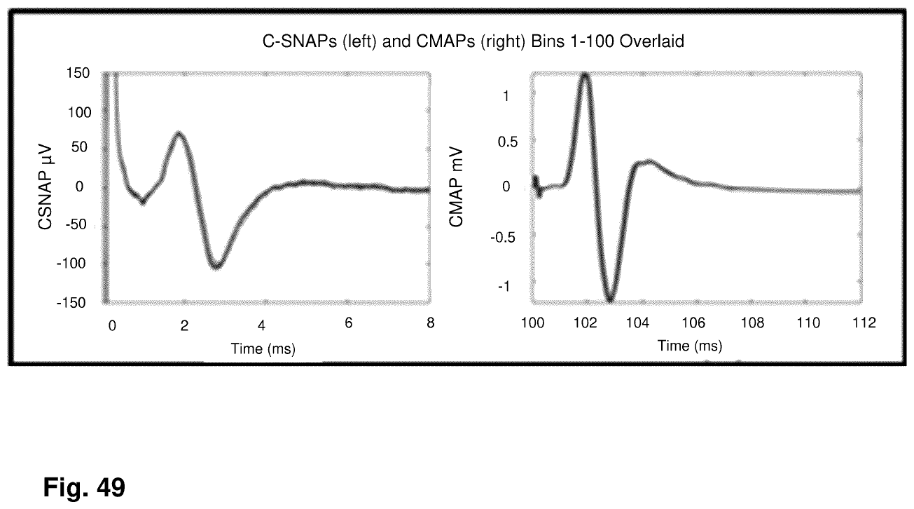

Hardware and Signal Processing for C-RPNI and MC-RPNI With reference to FIG. 48, the implant device 20a, the stimulation processing circuitry 22a, and the sensing processing circuitry 22b can be utilized and in communication with multiple C-RPNI constructs 500. While C-RPNI constructs 500 are shown in FIG. 48, MC-RPNI constructs can alternatively be used. The stimulation processing circuitry 22a and the sensing processing circuitry 22b can include a stimulation frontend and sensing frontend, respectively. The stimulation frontend and sensing frontend can include sufficient modules, processors, programming, memory, circuitry, and/or chips to carry out the functionality described in the present disclosure as being performed by the stimulation processing circuitry 22a and sensing processing circuitry 22b. As such, the terms stimulation frontend and sensing frontend can be used interchangeably with the terms stimulation processing circuitry 22a and sensing processing circuitry 22b, respectively.

In this way, dedicated hardware and signal processing can be used and performed for the stimulation and sensing electrodes of each C-RPNI construct, each having one or more electrodes for stimulation and one or more electrodes for sensing. Having separate stimulation and sensing frontends in an embedded system allows a wider selection of ASICS that are fully optimized for either low noise sensing or increased stimulation capabilities, as opposed to a system having single processing circuitry that provides functionality for both sensing and stimulation, such as the Intan RHS2116 chip described above.

As such, in accordance with the present disclosure, the implant device 20a can simultaneously stimulate and record signals from the same nerve at the same time.

With reference to FIG. 49, the illustrated data was collected by alternating stimulation and recording on the same nerve. This process was repeated 100 times to verify consistent signaling. The constant compound muscle action potentials (CMAPs) on the right graph of FIG. 49 illustrate that the muscle graft is not fatigued by simultaneous stimulation the dermal side of the C-RPNI, as shown by the compound sensory nerve action potentials (CSNAPs) on the left graft of FIG. 49.

The approaches and algorithms discussed above with reference to FIGS. 31-33 can be used with the embodiments for sensing and stimulation shown in FIGS. 47 and 48. For example, as described above with reference to FIGS. 31 and 32, sensing signals from the muscle graft 504 can be ignored while stimulation signals are transmitted to the dermal graft 502. In the embodiments of FIGS. 47 and 48, however, separate and independent processing circuitry/frontends (i.e., separate stimulation processing circuitry 22a and sensing processing circuitry 22b) and separate electrodes (i.e., separate sensing electrodes 14a, 14b, 14c and stimulation electrodes 30a, 30b, 30c) are used.

The use of independent electrodes and processing circuitry/frontends enables simultaneous interfacing, although artifacts from stimulation can still create interference. The embodiments of FIGS. 47 and 48, however, provide greater flexibility to mitigate artifacts. The embodiments of FIGS. 47 and 48 can also be used with the approach discussed above with reference to FIG. 33 to remove artifacts caused by stimulation by template/artifact extraction. With the use of a C-RPNI construct 500, the implant device 20 and sensing processing circuitry 22b can continue to sense and record signals from the same nerve that is being stimulated by the stimulation processing circuitry 22a. With a single electrode and RPNI configuration, the RPNI would need to be excluded from sensing/recording while signals are sensed and recorded from other RPNIs/channels.

With the use of separate processing circuitry from sensing and stimulation (22a, 22b), the implant device 20a can sense and record signals from all channels, e.g., all C-RPNIs or MC-RPNIs, while simultaneously, and independently, transmitting stimulation signals to the same C-RPNIs or MC-RPNIs. In accordance with the present disclosure, the implant device, stimulation processing circuitry 22a, and/or sensing processing circuitry 22b can be configured to switch control parameters during operating depending on whether stimulation is being performed simultaneously with sensing/recording. Under this approach, the sensing processing circuitry 22b can be programmed to implement a machine learning model with specific control parameters/weights that is configured and trained to receive motor control nerve signals from the sensing electrodes 14a, 14b, 14c and generate control signals to control, for example, a prosthetic. The machine learning model can include, for example, a neural network configured and trained with initial control parameters and weights that are then adjusted as the machine learning model and neural network are trained.

Under this approach, the sensing processing circuitry 22b can be configured to utilize different control parameters for generating control signals depending on whether the stimulation processing circuitry 22a is simultaneously transmitting sensory stimulation signals to the stimulation electrodes 30a, 30b, 30c. For example, the sensing processing circuitry 22b can be initialized with initial default control parameters. Once implanted within a patient and connected to a prosthetic device, the patient can be instructed to perform certain actions with the prosthetic. In this first phase of the process, the sensing processing circuitry 22b can sense and record the generated motor signals from the nerves within the muscle graft 504 via sensing electrodes 14a, 14b, 14c and generate control signals to control a prosthetic device. The control parameters used and stored by the sensing processing circuitry 22b can then be updated based on feedback with respect to the control motions of the prosthetic device. In this way, the first phase of the process generates a first set of control parameters for use during time periods when the stimulation processing circuitry 22a is not simultaneously providing stimulation signals to the skin graft 502 portion of the C-RPNI construct 500.

In a second phase, the patient can be instructed to perform the same actions with the prosthetic. However, in this second phase of the process, the stimulation processing circuitry 22a is configured to simultaneously provide stimulation signals to the skin graft 502 via the stimulation electrodes 30a, 30b, 30c while the sensing processing circuitry 22b senses and records the generated motor signals from the nerves within the muscle graft 504 via sensing electrodes 14a, 14b, 14c and generates control signals to control a prosthetic device. The control parameters used and stored by the sensing processing circuitry 22b can then be updated based on feedback with respect to the control motions of the prosthetic device to generate a second set of control parameters. In this way, the second phase of the process generates a second set of control parameters for use during time periods when the stimulation processing circuitry 22a is simultaneously providing stimulation signals to the skin graft 502 portion of the C-RPNI construct 500. This second set of control parameters beneficially and automatically account for any artifacts generated in the C-RPNI construct 500 due to stimulation signals transmitted to the stimulation electrodes by the stimulation processing circuitry 22a.

As a result of the above first and second phases of the training process, the sensing processing circuitry 22b can store and utilize two separate sets of control parameters including a first set of control parameters for use when the stimulation processing circuitry 22a is not providing stimulation signals to the skin graft 502 of the C-RPNI construct 500 and a second set of control parameters for use when the stimulation processing circuitry 22a is providing stimulation signals to the skin graft 502 of the C-RPNI construct 500.

In this way, the sensing processing circuitry 22b can utilize a baseline “stimulation off” set of control parameters for receiving signals from the sensing electrodes 14a, 14b, 14c while stimulation is not being performed and a set of “stimulation on” control parameters for receiving signals from the sensing electrodes 14a, 14b, 14c while stimulation is being performed. In addition, multiple sets of “stimulation on” control parameters can be generated and utilized to account for different stimulation combinations. The “stimulation on” control parameters can all the implant device 20a to maintain control of the prosthetic device while in the presence of any stimulation artifacts caused by the stimulation processing circuitry 22a. This may approach, for example, may be beneficially avoid the use of alternating time windows, as with other approaches described above with reference to FIGS. 31 and 32, which decrease the update/sensing rate of the sensing processing circuitry 22b by half. This approach may also beneficially avoid the use of template matching algorithms and the precise timing required by the approach described above with respect to FIG. 33.

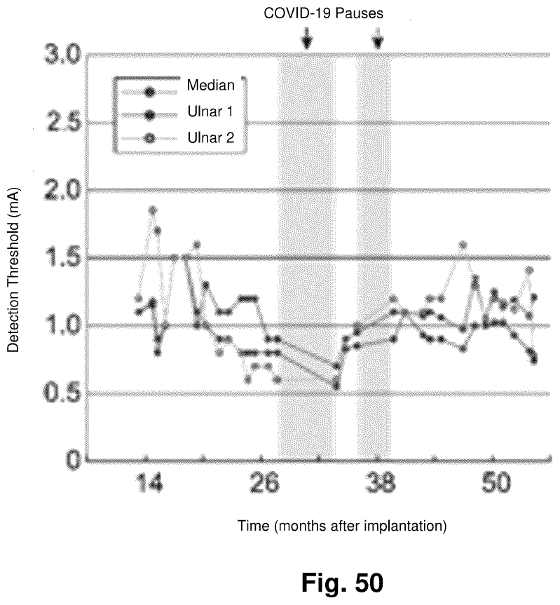

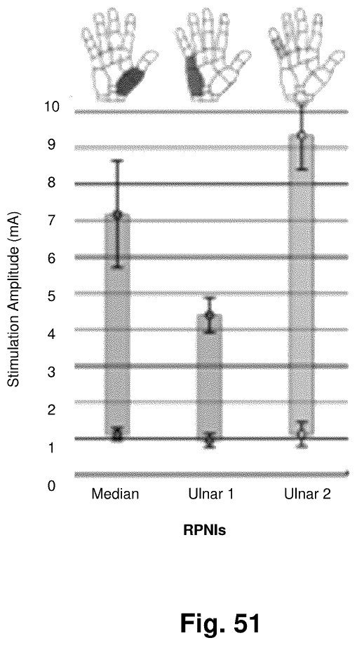

Stimulation of RPNIs facilitates kinesthetic sensations (e.g. movement) and cutaneous sensations. Further, FIG. 50 shows stable sensory detection thresholds for a patient's RPNIs over time. FIG. 51 shows hand maps depicting the somatotopic accuracy of referred sensation. However, the elicited cutaneous sensations were not reported as feeling natural and often described as tingling, buzzing, etc. This may be due to the different sensory end organ composition compared to skin and inability to separate the sensory fibers from the muscle fibers in the RPNI.

The C-RPNI is composed of a transected nerve implanted between a muscle graft and a dermal graft. As discussed above, this allows sensory nerves to reinnervate the dermal side and motor nerves to reinnervate the muscle side. Thus, motor signaling can be facilitated through electromyography (EMG) recordings from the muscle component, but can also independently facilitate sensory feedback through stimulation of the dermal component. C-RPNI can be used to either record efferent motor signals or provide afferent sensory feedback. However, many nerves in the body are mixed motor and sensory nerves, particularly at higher levels of amputations. In these cases, the use of multiple electrodes in a C-RPNI provides an interface for truly independent and near-simultaneous sensory feedback and motor control.

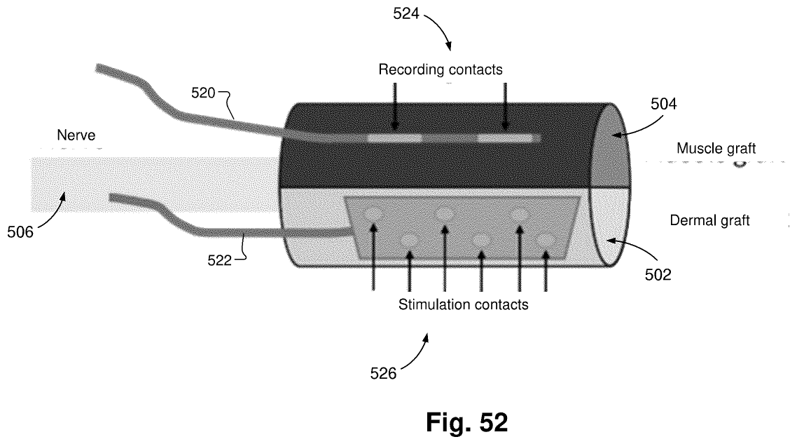

With reference to FIG. 52, the bidirectional interface consists of a sensing electrode 520 implanted in muscle tissue 504. The sensing electrode 520 can be a single monopolar or bipolar electrode, as shown in FIG. 52. For example, the sensing electrode 520 can include recording contacts 524, such as the two recording contacts 524 shown in FIG. 52. Alternatively, the sensing electrode 520 can include multiple electrodes or high-resolution sensing electrodes. One or more stimulating electrodes can also be implanted. For example, stimulating electrode 522 can be a multi-contact electrode with multiple stimulation contacts 526, as shown in FIG. 52. A multi-contact electrode with more than two smaller electrode contacts is described in detail in U.S. Pat. No. 10,779,963, which is incorporated herein by reference in its entirety. Multiple sensing contacts selectively activate nearby fibers of the dermal graft, which enables finer resolution control of the perceived location and quality of sensation. The stimulating electrode 522 may also be a monopolar or bipolar electrode with larger contacts. However, a stimulating electrode 522 with larger contacts may inadvertently activate the muscle tissue.

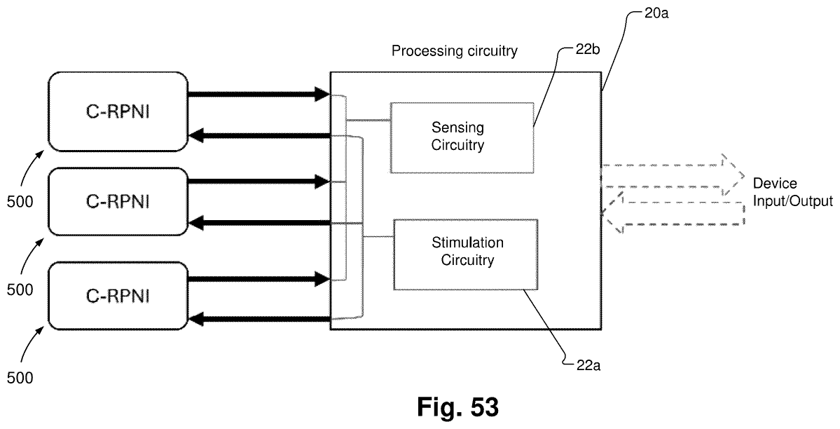

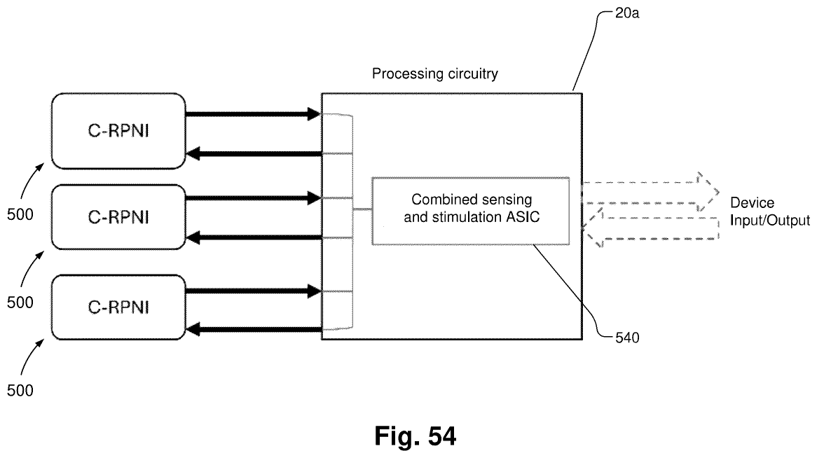

With reference to FIG. 53, an implant device 20a can use dedicated sensing ASICs optimized for low-noise sensing and dedicated stimulation ASICs. For example, the implant device 20a can include stimulation processing circuitry 22a and sensing processing circuitry 22b on separate ASICs. Alternatively, with reference to FIG. 54, combined sensing and stimulation processing circuitry 540 can also be used and integrated on a single ASIC. Typically, combined ASICs minimize size, with lesser performance and fewer capabilities as compared with implementations with separate ASICs for sensing and stimulation.

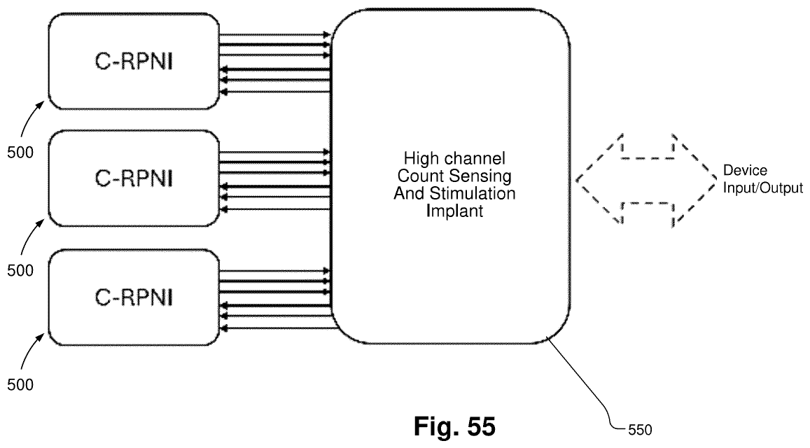

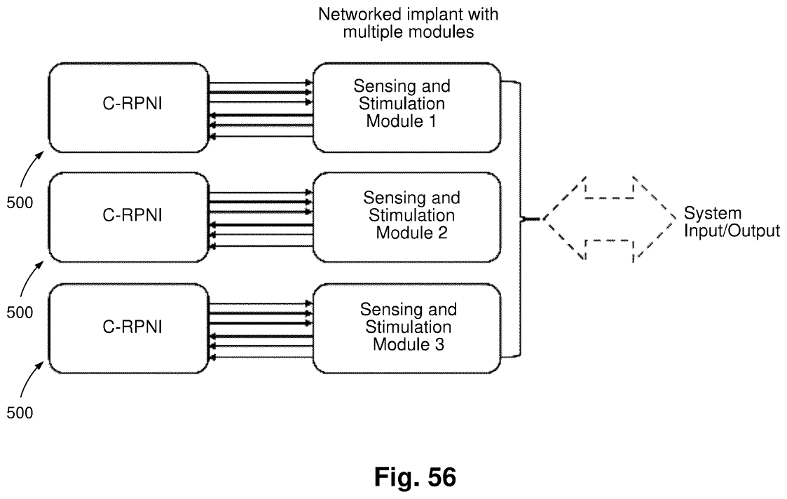

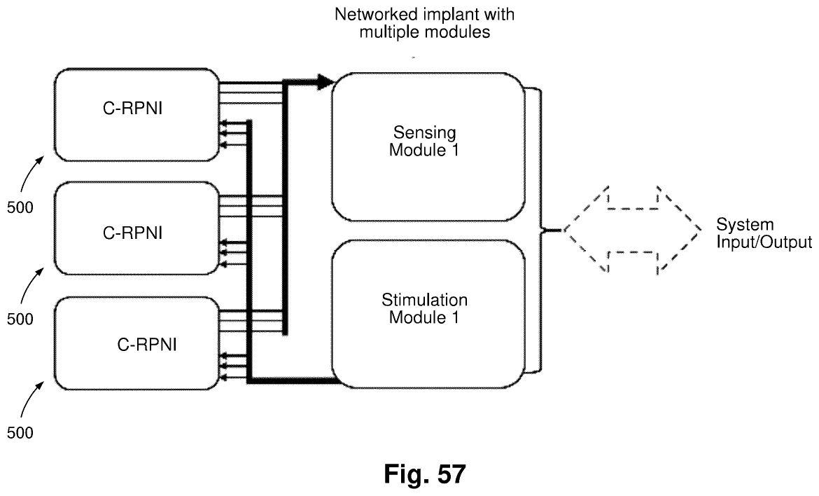

With reference to FIG. 55, the processing circuitry can be contained in a single implant device 550. For example, as shown in FIG. 55, the single implant device 550 is a high channel count sensing and stimulation implant device. With reference to FIG. 56, the processing circuitry can be distributed across a network of multiple implanted devices/modules in a system. Existing embodiments of networked implants could be leveraged. For example, wireless Body Area Networks (BAN) or a wired networked implant can be used. The network architecture can be used to increase the total system channel count and may be organized by geography, as shown in FIG. 56, or by function, as shown in FIG. 57. Each module can include the hardware, circuitry, memory, and processors described above as being included in the processing circuitry 22. In addition, the modules can include sufficient communication hardware and software stored in memory to enable communication between the modules. In this way, the modules can receive input from the various input channels of the C-RPNIs 500 and coordinate to generate a system output to control a prosthetic device. FIG. 56, for example, shows three modules Module 1, Module 2, and Module 3 that each include sensing and stimulation processing circuitry to perform sensing and stimulation functions via multiple sensing and stimulation channels connected to each C-RPNI 500. FIG. 57, on the other hand, includes a sensing module 1 and a stimulation module 1. The sensing module 1 is connected to a bus connected with the sensing channels of each C-RPNI 500, while the stimulation module 1 is connected to a separate bus connected with the stimulation channel of each C-RPNI 500. Alternatively, a single bus can be used for both modules and all C-RPNIs.

With each of the embodiments, different techniques can be applied to mitigate stimulation artifact for a true bi-directional interface. For example, one technique includes alternating stimulation and recording windows in rapid succession, as discussed above. This technique enables near-simultaneous afferent and efferent signaling. To minimize noticeable delays in a prosthetic control system, each block of the stimulation plus recording windows should total less than 200 ms (ideally less than 100 ms), although other time periods can be used. The stimulation and record windows do not need to be equal length. Rather, the stimulation window can be minimized as much as possible while eliminating corruption of efferent signal measurements due to artifacts. If other signal filtering or artifact subtraction techniques are successful in removing the stimulating artifact, the stimulation window can be eliminated entirely for true simultaneous control.

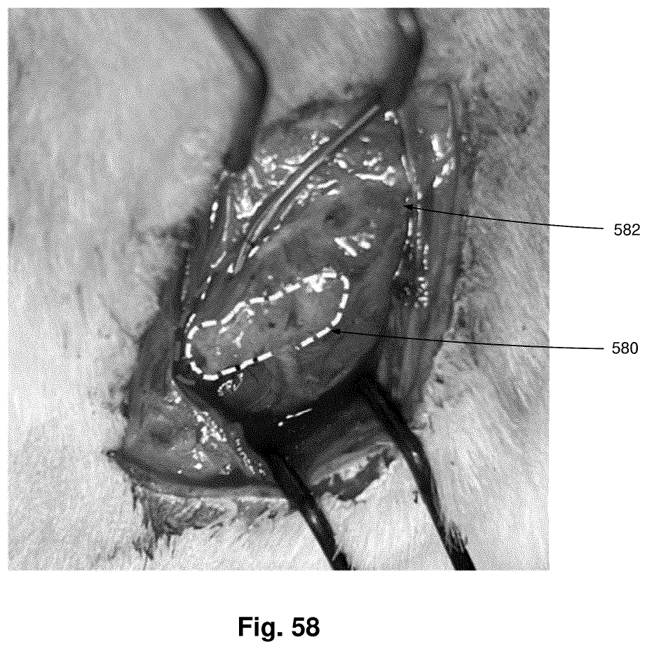

As shown in FIG. 58, a photograph of a C-RPNI construct is shown and includes a transected nerve implanted in between a dermal graft 580 and a muscle graft 582.

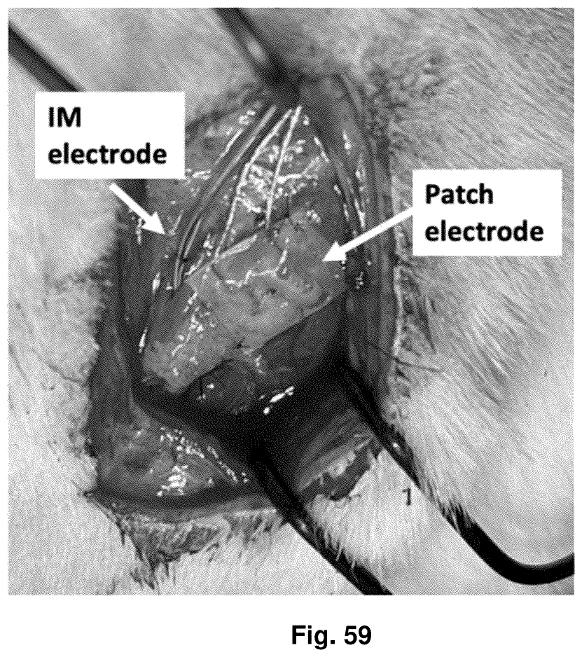

FIG. 59 shows a photograph of an electrodiagnostic setup wherein afferent signaling was generated and recorded by stimulating the dermal graft with a patch electrode and recording afferent activity with a nerve cuff on the proximal CP nerve. Muscle signaling was generated and recorded by stimulating the proximal CP nerve with the nerve cuff and recording EMG signals with a bipolar intramuscular electrode in the muscle graft.

Within each C-RPNI, the dermal graft naturally attracts sensory fibers, which were interfaced using a dedicated stimulation electrode and stimulation front-end, i.e., stimulation processing circuitry. The muscle graft is naturally reinnervated by motor fibers which were interfaced with using dedicated recorded electrodes and a sensing front-end, i.e., sensing processing circuitry.

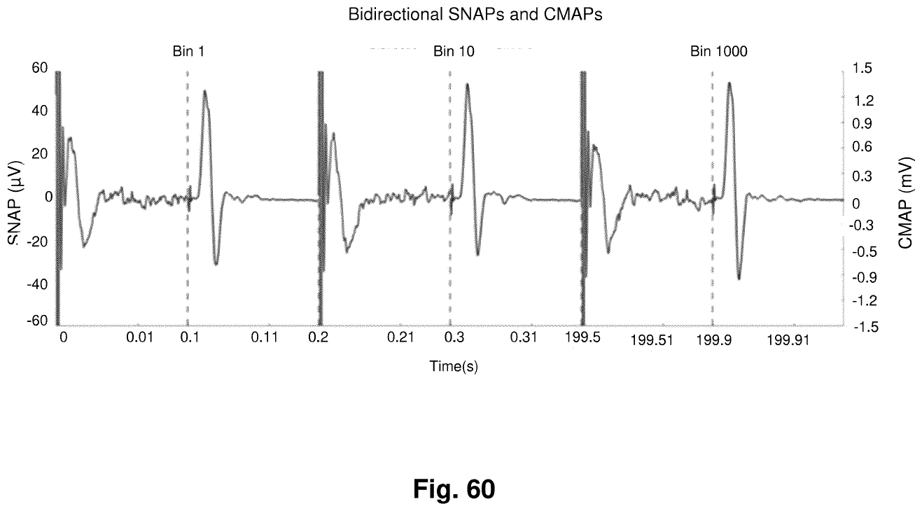

The C-RPNIs enable simultaneously stimulation and recording from the same nerve at the same time. With reference to FIG. 60, a graph illustrates data that was collected by alternating stimulation and recording on the same nerve. This was repeated 1,000 times to verify consistent signaling. The constant CMAP illustrated in FIG. 60 establishes that the muscle was not fatigued by stimulating the dermal side of the C-RPNI.

Composite Cuff Regenerative Peripheral Nerve Interface (CC-RPNI) An ideal control system for powered exoskeletons would respond to the user's intent by harnessing signals directly from the nervous system. A direct interface to capture movement intent from nerves is key as the exoskeleton must produce movements with minimal latency to coordinate with functioning muscles that provide partial mobility. Sensory feedback is an important component of control for both the upper and lower extremity. Patients with intact nerves may retain only partial motor function and partial sensory feedback on their affected limb. A dual interface is therefore needed to fully restore function in these cases. The composite cuff regenerative peripheral nerve interface (CC-RPNI) is an ideal interface for this dual function as the CC-RPNI includes a dermal component that can be reinnervated by sensory fibers for sensory feedback and a muscle component that can amplify motor signals to detect motor intent.

The electrode interface, implantable devices, and processing circuitry for CC-RPNI constructs is similar to that described above with respect to the bi-directional interface with electrodes in different tissues for composite regenerative peripheral nerve interface (C-RPNI). In the case of CC-RPNI constructs, however, the multi-tissue graft is created on an intact nerve to preserve any remaining downstream functions.

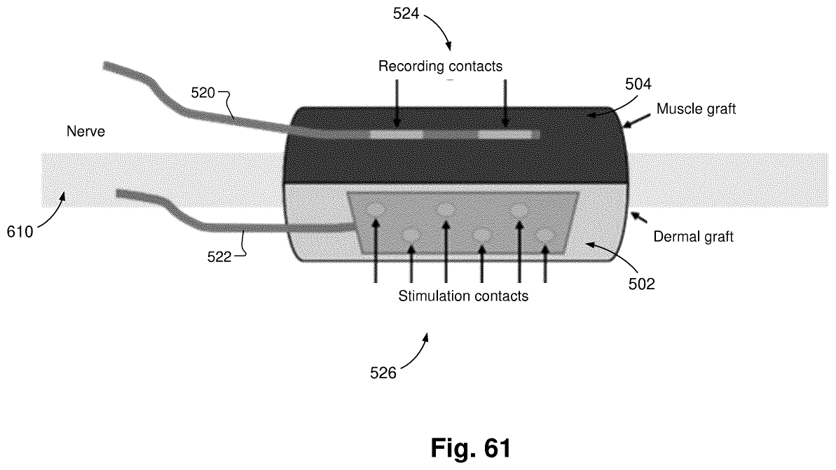

As shown in FIG. 61, the CC-RPNI construct includes a free muscle graft 504 and a free dermal graft 502 secured circumferentially to an intact peripheral nerve 610. The free muscle graft 504 and the free dermal graft 502 then become reinnervated by the contained nerve and can amplify the nerve signals to facilitate reliable and accurate detection of motor intent and to communicate sensory feedback.

Similar to the discussion above with respect to C-RPNI construct discussed above, the bidirectional interface consists of a sensing electrode 520 implanted in muscle tissue 504. The sensing electrode 520 can be a single monopolar or bipolar electrode, as shown in FIG. 61. For example, the sensing electrode 520 can include recording contacts 524, such as the two recording contacts 524 shown in FIG. 61. Alternatively, the sensing electrode 520 can include multiple electrodes or high-resolution sensing electrodes. One or more stimulating electrodes can also be implanted. For example, a stimulating electrode 522 can be a multi-contact electrode with multiple stimulation contacts 526, as shown in FIG. 61. A multi-contact electrode with more than two smaller electrode contacts is described in detail in U.S. Pat. No. 10,779,963, which is incorporated herein by reference in its entirety. Multiple sensing contacts selectively activate nearby fibers of the dermal graft, which enables finer resolution control of the perceived location and quality of sensation. The stimulating electrode 522 may also be a monopolar or bipolar electrode with larger contacts. However, a stimulating electrode 522 with larger contacts may inadvertently activate the muscle tissue.

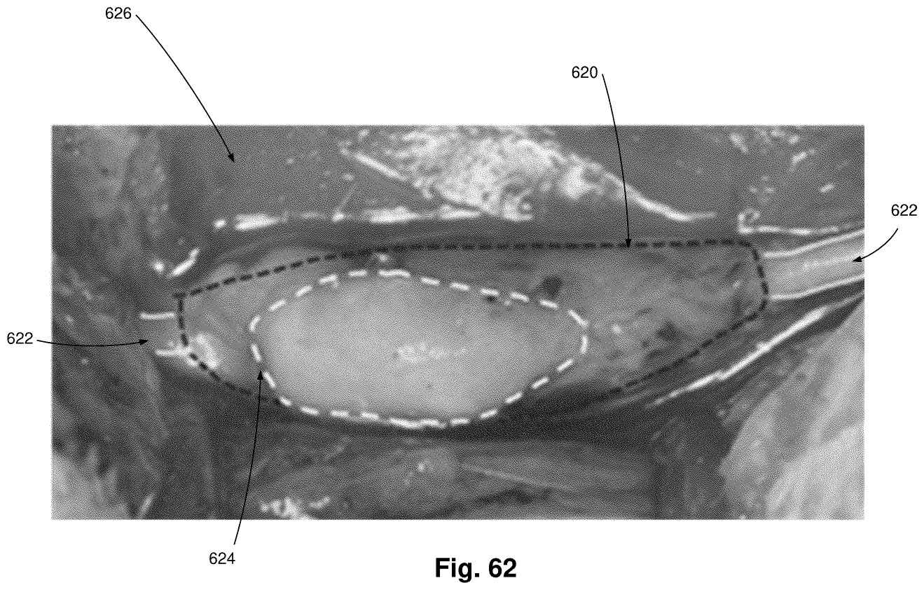

With reference to FIG. 62, a photograph of a CC-RPNI construct 626 is shown. The CC-RPNI construct 626 includes a muscle graft 620 that is circumferentially wrapped around an intact nerve 622. A dermal graft 624 is secured to the muscle graft 620 around the intact nerve 622.

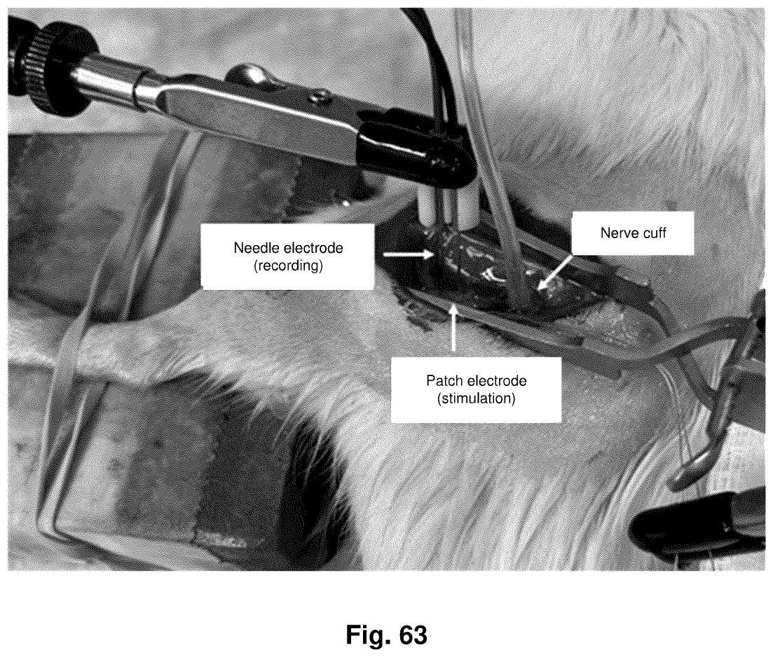

With reference to FIG. 63, afferent signaling was generated and recorded by stimulating the dermal graft with a patch electrode and recording afferent activity with a nerve cuff on the proximal CP nerve. Muscle signaling was generated and recorded by stimulating the proximal CP nerve with the nerve cuff and recording EMG signals with fine wire electrodes in the muscle graft.

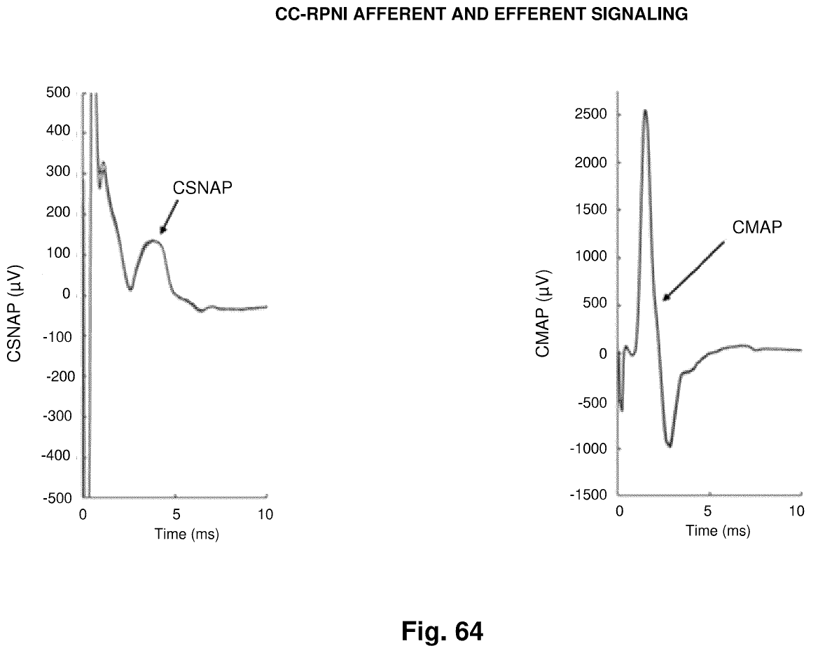

With reference to FIG. 64, a graph illustrates CC-RPNI afferent and efferent signaling. The left-hand side of the graph of FIG. 64 illustrates successful reinnervation of the dermal portion of the graft for afferent sensory signaling. Electrically stimulating the dermis graft produces a CSNAP recorded upstream from the nerve cuff. The right-hand side of the graph of FIG. 64 illustrates successful reinnervation of the muscle portion of the graft for efferent motor signaling. Stimulating the nerve cuff produces a downstream CMAP recorded from the muscle.

### Muscle Cuff and Skin Regenerative Peripheral Nerve Interface (RPNI) with Electrodes in Muscle and Skin

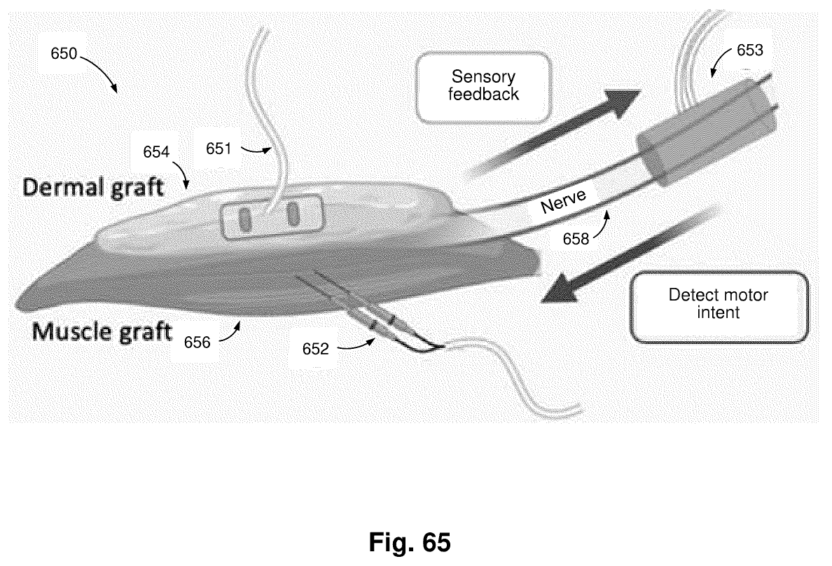

Common implanted electrodes have large contact relative to the size of the RPNI and C-RPNI constructs. Stimulating a construct with a large electrode may produce large field of activation that unintentionally activates muscle and nerve fibers. With reference to FIG. 65, a setup for electrophysiology experiments on a C-RPNI 650 construct is illustrated and includes a dermal graft 654 and a muscle graft 656 attached to a nerve 658. With the illustrated setup, a stimulating dermal electrode 651 is configured to transmit stimulation electrical signals to the dermal graft 654 and a recording muscle electrode 652 is configured to record electrical signals from the muscle graft 656. A stimulating and recording nerve cuff electrode 653 is configured to detect record sensor feedback electrical signals from the nerve 658, such as signals transmitted through the dermal graft 654. The stimulating and recording nerve cuff electrode 653 is also configured to transmit direct motor intent electrical signals to the nerve 658. The recording muscle electrode 652 can then record electrical signals that were transmitted from the stimulating and recording nerve cuff electrode 653 to the nerve 658 and then to the muscle graft 656.

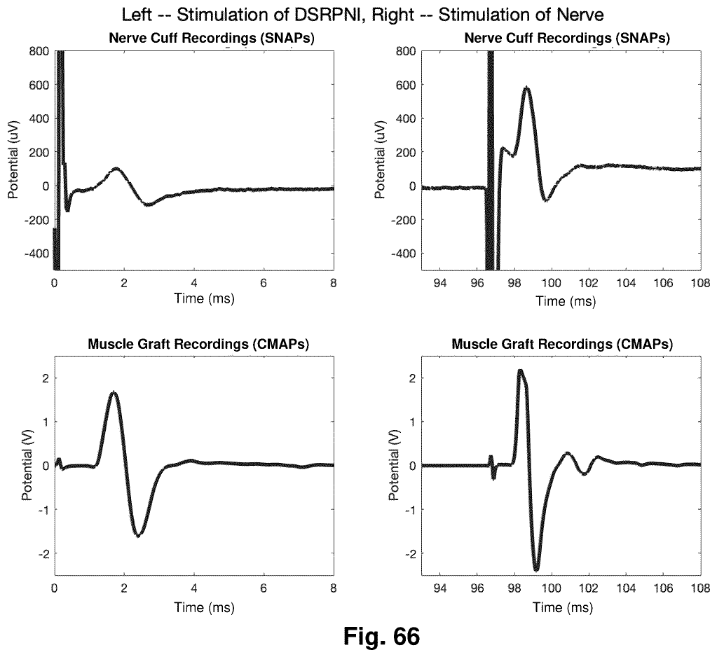

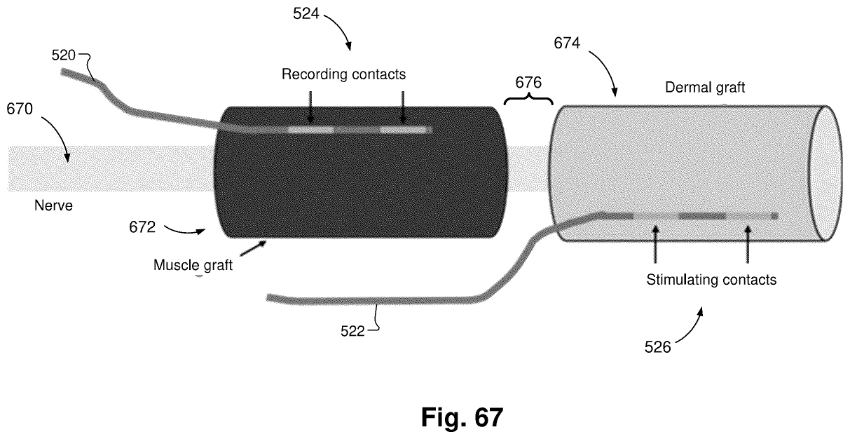

With reference to FIG. 66, signal recordings using the setup illustrated in FIG. 65 are shown. The graphs on the left-hand side of FIG. 66 illustrate the nerve cuff recordings and muscle graft recordings with stimulation of the dermal graft 654 using the stimulating dermal electrode 651. For example, the afferent nerve action potential generated by stimulating the dermal graft 654 is shown in the top left graph of FIG. 66. However, the muscle graft 656 is also activated by the electrical stimulus, as evidenced by the large amplitude compound motor action potential, shown in the bottom left graph of FIG. 66. The magnitude of this activation shown in the bottom left graph of FIG. 66 is comparable to generated activity from efferent nerve signaling, as shown in the bottom right graph of FIG. 66. The graphs on the right-hand side of FIG. 66 illustrate the recordings when the nerve 658 is stimulated by the stimulating and recording nerve cuff electrode 653. As shown in the bottom left and bottom right graphs of FIG. 66, the muscle graft recordings are similar when stimulation is provided by the nerve 658 and when stimulation is provided to the dermal graft 654. As discussed above, near-simultaneous interfacing can be achieved without fatiguing the muscle tissue. However, there may be undesirable clinical effects from this unintended activation such as the production of uncontrolled kinesthetic sensations when the intent is to produce only a controlled somatosensation like tactile feedback.

Uncontrolled sensation may confound and degrade the quality of sensory feedback.

Additionally undesirable clinical effects may include muscle activation produced by electrical stimulation, which may confound volitional movement commands. As discussed above, a multi-contact sensing electrode may be used to mitigate this issue.

To address these issues, the sensing and stimulating interfaces can be geometrically separated to enable the use of larger, simpler electrodes that can stimulate afferent sensory fibers without confounding muscle activation.

With reference to FIG. 67, a construct is shown having a single tissue cuff circumferentially surrounding around a portion of nerve 670 and an RPNI with a different tissue located at the end of the nerve 670. For example, a free muscle graft cuff 672 is circumferentially attached around the nerve 670, while a free dermal graft 674 is attached to the end of the nerve 670. A sensing electrode 520 is implanted in the muscle tissue 672. The sensing electrode 520 can be a single monopolar or bipolar electrode, as shown in FIG. 67. For example, the sensing electrode 520 can include recording contacts 524, such as the two recording contacts 524 shown in FIG. 67. Alternatively, the sensing electrode 520 can include multiple electrodes or high-resolution sensing electrodes. One or more stimulating electrodes can also be implanted. For example, a stimulating electrode 522 can be a multi-contact electrode implanted in the dermal graft 674 with multiple stimulation contacts 526, as shown in FIG. 67. A multi-contact electrode with more than two smaller electrode contacts is described in detail in U.S. Patent No. US10,779,963, which is incorporated herein by reference in its entirety. Multiple sensing contacts selectively activate nearby fibers of the dermal graft, which enables finer resolution control of the perceived location and quality of sensation. The stimulating electrode 522 may also be a monopolar or bipolar electrode with larger contacts. However, a stimulating electrode 522 with larger contacts may inadvertently activate the muscle tissue.

As shown in FIG. 67, the free muscle graft 672 is physically and geometrically separated on the nerve 670, creating a gap 676 and physical separation between the muscle graft 672 and the dermal graft 674 along the axis of the nerve 670. The gap 676 between the two tissues geometrically isolates the interfaces with minimized signaling interference. In this example, a muscle cuff is created with the muscle graft 672 on the intact portion of the nerve proximal to a dermal graft 674 created at the end of the nerve, with the muscle graft 672 and dermal graft 674 being separated on the axis of the nerve 670 by the physical gap 676. Motor fibers reinnervate the motor end plates on the muscle graft 672 of the muscle cuff, while sensory fibers reinnervate the dermal graft 674 at the end of the nerve 670. Motor fibers at the end of the nerve do not have a reinnervation target in the dermal graft and are pruned.

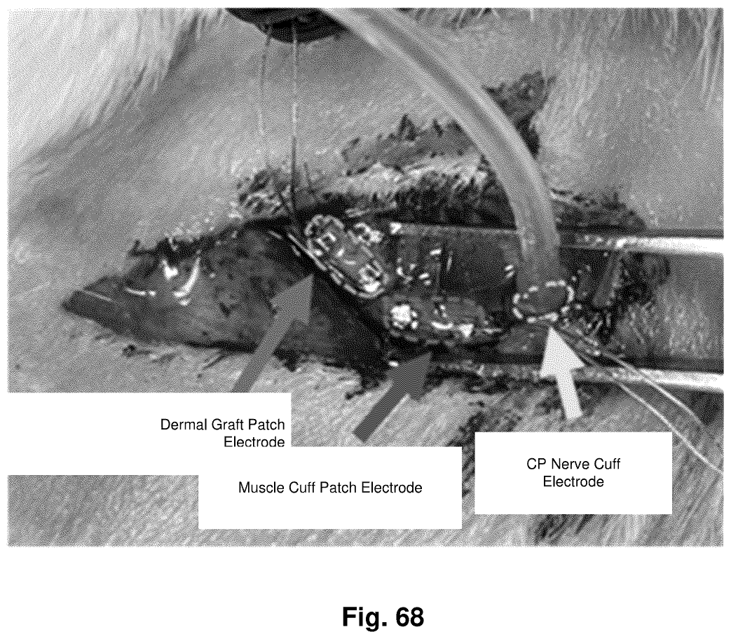

With reference to FIG. 68, the construct of this embodiment geographically separates the motor and sensory interfaces along the axis of the nerve. This greatly improves isolation between the motor and sensory portions of the tissue interfaces and enables independent activation of sensory and motor functions with independent electrodes. Amplified efferent motor signals are recorded from the muscle cuff portion of the construct and afferent sensory feedback is delivered through the skin graft on the end of the severed nerve 670.

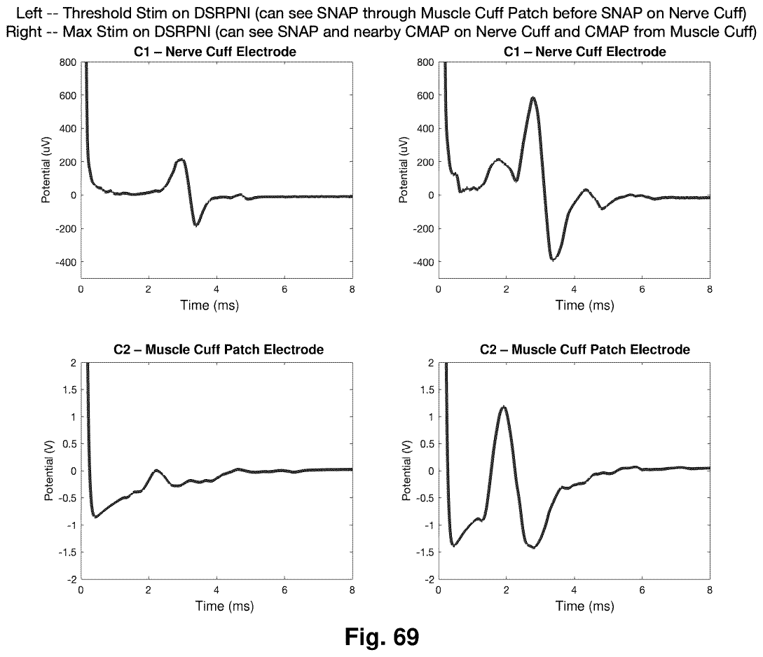

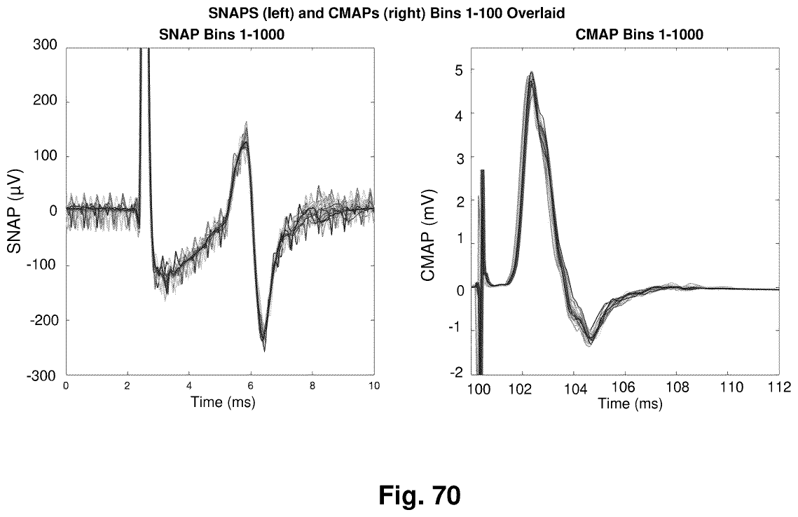

With reference to FIG. 69, signal recordings using the constructs of FIGS. 67 and 68 are shown. For example, a graph of the afferent nerve action potential generated by stimulating the dermal graft 674 is shown in the top left graph of FIG. 69. Unlike other constructs, there is no observed motor unit action potential in the muscle graft 672, as shown in the bottom left graph of FIG. 69, establishing isolation. In other words, with the constructs of FIGS. 67 and 68 of the present disclosure, the muscle graft 672 is isolated from stimulation signals provided to the dermal graft 674. However, efferent motor signaling from the nerve does produce a large amplitude motor unit action potential in the muscle cuff tissue, as shown in the bottom right graph of FIG. 69. Similar to the C-RPNI, the stability of the skin RPNI and muscle cuff were also validated with 1000 repetitions of afferent and efferent signaling, as shown in the graphs of FIG. 70.

### Non-Limiting Discussion of Terminology

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure. Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

As used herein, the term circuitry may refer to, be part of, or include: an Application Specific Integrated Circuit (ASIC); an electronic circuit; a combinational logic circuit; a field programmable gate array (FPGA); a processor (shared, dedicated, or group) that executes code; other suitable hardware components that provide the described functionality; or a combination of some or all of the above, such as in a system-on-chip. The term circuitry may include memory (shared, dedicated, or group) that stores code executed by the processor.

The term code, as used above, may include software, firmware, and/or microcode, and may refer to programs, routines, functions, classes, and/or objects. The term shared, as used above, means that some or all code from multiple circuitries may be executed using a single (shared) processor. In addition, some or all code from multiple circuitries may be stored by a single (shared) memory. The term group, as used above, means that some or all code from single circuitry may be executed using a group of processors. In addition, some or all code from single circuitry may be stored using a group of memories.

The apparatuses and methods described herein may be implemented by one or more computer programs executed by one or more processors. The computer programs include processor-executable instructions that are stored on a non-transitory tangible computer readable medium. The computer programs may also include stored data. Non-limiting examples of the non-transitory tangible computer readable medium are nonvolatile memory, magnetic storage, and optical storage.

The headings (such as “Background” and “Summary”) and sub-headings used herein are intended only for general organization of topics within the present disclosure, and are not intended to limit the disclosure or any aspect thereof. In particular, subject matter disclosed in the “Background” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The disclosure of all patents and patent applications referenced or cited in this disclosure are incorporated by reference herein.

The description and specific examples, while indicating features and embodiments, are intended for purposes of illustration only and are not intended to limit the scope of the disclosure. Moreover, recitation of multiple embodiments having stated features is not intended to exclude other embodiments having additional features, or other embodiments incorporating different combinations of the stated features. Specific examples are provided for illustrative purposes of how to make and use the described methods, systems, and compositions and, unless explicitly stated otherwise, are not intended to be a representation that given embodiments have, or have not, been made or tested.

As used herein, the words “prefer” or “preferable” refer to embodiments that afford certain benefits, under certain circumstances. However, other embodiments may also be preferred, under the same or other circumstances.

Furthermore, the recitation of one or more preferred embodiments does not imply that other embodiments are not useful, and is not intended to exclude other embodiments from the scope of the disclosure.

As used herein, the word “include,” and its variants, is intended to be non-limiting, such that recitation of items in a list is not to the exclusion of other like items that may also be useful in the methods, systems, materials, compositions, and devices described. Similarly, the terms “can” and “may” and their variants are intended to be non-limiting, such that recitation that an embodiment can or may comprise certain elements or features does not exclude other embodiments that do not contain those elements or features.

Although the open-ended term “comprising,” as a synonym of non-restrictive terms such as including, containing, or having, is used herein to describe and claim embodiments of the present disclosure, embodiments may alternatively be described using more limiting terms such as “consisting of” or “consisting essentially of.” Thus, for any given embodiment reciting materials, components, or process steps, the present disclosure also specifically includes embodiments consisting of, or consisting essentially of, such materials, components, or processes excluding additional materials, components, or processes (for consisting of) and excluding additional materials, components, or processes affecting the significant properties of the embodiment (for consisting essentially of), even though such additional materials, components, or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, ranges are, unless specified otherwise, inclusive of endpoints and include disclosure of all distinct values and further divided ranges within the entire range. Thus, for example, a range of “from A to B” or “from about A to about B” is inclusive of A and of B. Disclosure of values and ranges of values for specific parameters (such as temperatures, molecular weights, weight percentages, etc.) are not exclusive of other values and ranges of values useful herein. The use of the term “about” with respect to a range, value, or threshold is to be considered in the context of the range, value, or threshold, as understood by one of ordinary skill in the art. To the extent, the range, value, or threshold cannot be determined from the context, the use of the term “about” can correspond to a ten to fifteen percent range. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

What is claimed is:

1. A system comprising:
an implant device having sensing processing circuitry configured to receive electrical signals from a composite tissue graft surgically attached to surround a nerve of a subject and having stimulating processing circuitry configured to transmit electrical signals to the composite tissue graft, the composite tissue graft including a muscle graft portion and a dermal graft portion, the composite tissue graft being surgically attached to the subject such that the composite tissue graft is entirely surrounded by and in direct contact with non-grafted tissue of the subject, the muscle graft potion and the dermal graft portion each being an autograft of tissue harvested from the subject, devascularized, and deinnervated prior to being surgically attached to the subject, the nerve having reinnervated the muscle graft portion and the dermal graft portion of the composite tissue graft subsequent to the composite tissue graft being surgically attached to the nerve;
wherein:
at least one sensing electrode is attached to, and in electrical communication with, the muscle graft portion of the composite tissue graft;
at least one stimulating electrode is attached to, and in electrical communication with, the dermal graft portion of the composite tissue graft;
the sensing processing circuitry is configured to receive electrical signals from the at least one sensing electrode, process the received electrical signals from the at least one sensing electrode, generate processed motor signal data corresponding to electrical signals received from the at least one sensing electrode, and to transmit the processed motor signal data to a prosthetic controller, the prosthetic controller being configured to control a prosthetic device based on the processed motor signal data transmitted from the sensing processing circuitry of the implant device; and
the stimulating processing circuitry is configured to receive sensory feedback data from the prosthetic controller, generate processed sensory signal data based on the sensory feedback data, and transmit electrical signals to the at least one stimulating electrode corresponding to the processed sensory signal data.

2. The system of claim 1, wherein the at least one sensing electrode has a diameter less than 1.5 millimeters, a recording contact length of 1 to 2 centimeters, and a bipolar inter-contact spacing less than 2 centimeters.

3. The system of claim 1, wherein the at least one sensing electrode has a recording contact length of less than 2 millimeters.

4. The system of claim 1, wherein the at least one sensing electrode is a high-resolution electrode having eight contacts that each have a recording contact length of 0.2 millimeters or less.

5. The system of claim 1, wherein the at least one stimulating electrode is a multi-contact electrode having more than two electrode contacts configured to transmit electrical signals to the dermal graft portion of the composite tissue graft.

6. The system of claim 1, wherein the sensing processing circuitry is included on a first application-specific integrated circuit (ASIC) of the implant device and the stimulating processing circuitry is included on a second ASIC of the implant device.

7. The system of claim 1, wherein:
the sensing processing circuitry is configured to receive additional electrical signals received from at least one additional sensing electrode attached to an additional composite tissue graft, the additional composite tissue graft being surgically attached to surround an additional nerve of the subject, the additional composite tissue graft including an additional muscle graft portion and an additional dermal graft portion, the at least one additional sensing electrode being attached to the additional muscle graft portion of the additional composite tissue graft, the additional composite tissue graft being surgically attached to the subject such that the additional composite tissue graft is entirely surrounded by and in direct contact with non-grafted tissue of the subject, the additional muscle graft potion and the additional dermal graft portion each being an additional autograft of tissue harvested from the subject, devascularized, and deinnervated prior to being surgically attached to the subject, the additional nerve having reinnervated the additional muscle graft portion and the additional dermal graft portion of the additional composite tissue graft subsequent to the additional composite tissue graft being surgically attached to the additional nerve; and
the sensing processing circuitry is configured to receive the additional electrical signals from the at least one additional sensing electrode, process the additional electrical signals received from the at least one additional sensing electrode, generate additional processed motor signal data corresponding to the additional electrical signals received from the at least one additional sensing electrode, and to transmit the additional processed motor signal data to the prosthetic controller, the prosthetic controller being configured to control the prosthetic device based on the additional processed motor signal data transmitted from the additional sensing processing circuitry of the implant device.

8. The system of claim 1, wherein:
the stimulating processing circuitry is configured to transmit additional electrical signals to at least one additional sensing electrode attached to an additional composite tissue graft, the additional composite tissue graft being surgically attached to surround an additional nerve of the subject, the additional composite tissue graft including an additional muscle graft portion and an additional dermal graft portion, the at least one additional sensing electrode being attached to the additional dermal graft portion of the additional composite tissue graft, the additional composite tissue graft being surgically attached to the subject such that the additional composite tissue graft is entirely surrounded by and in direct contact with non-grafted tissue of the subject, the additional muscle graft potion and the additional dermal graft portion each being an additional autograft of tissue harvested from the subject, devascularized, and deinnervated prior to being surgically attached to the subject, the additional nerve having reinnervated the additional muscle graft portion and the additional dermal graft portion of the additional composite tissue graft subsequent to the additional composite tissue graft being surgically attached to the additional nerve; and
the stimulating processing circuitry is configured to receive additional sensory feedback data from the prosthetic controller, generate additional processed sensory signal data based on the additional sensory feedback data, and transmit the additional electrical signals to the at least one additional stimulating electrode corresponding to the additional processed sensory signal data.

9. The system of claim 1, wherein the sensing processing circuitry is included within a first module and the stimulating processing circuitry is included within a second module, the first and second module being in wireless communication with each other via at least one of a wireless body area network (BAN) or a wired network implanted within the subject.

10. The system of claim 1, wherein the electrical signals received from the muscle portion of the composite tissue graft have a voltage amplitude of greater than or equal to about 150 microvolts.

11. A method comprising:
receiving, with sensing processing circuitry of an implant device, electrical signals from a composite tissue graft surgically attached to surround a nerve of a subject;
transmitting, with stimulating processing circuitry of the implant device, electrical signals to the composite tissue graft, the composite tissue graft including a muscle graft portion and a dermal graft portion, the composite tissue graft being surgically attached to the subject such that the composite tissue graft is entirely surrounded by and in direct contact with non-grafted tissue of the subject, the muscle graft potion and the dermal graft portion each being an autograft of tissue harvested from the subject, devascularized, and deinnervated prior to being surgically attached to the subject, the nerve having reinnervated the muscle graft portion and the dermal graft portion of the composite tissue graft subsequent to the composite tissue graft being surgically attached to the nerve;
wherein:
at least one sensing electrode is attached to, and in electrical communication with, the muscle graft portion of the composite tissue graft;
at least one stimulating electrode is attached to, and in electrical communication with, the dermal graft portion of the composite tissue graft;
the sensing processing circuitry is configured to receive electrical signals from the at least one sensing electrode, process the received electrical signals from the at least one sensing electrode, generate processed motor signal data corresponding to electrical signals received from the at least one sensing electrode, and to transmit the processed motor signal data to a prosthetic controller, the prosthetic controller being configured to control a prosthetic device based on the processed motor signal data transmitted from the sensing processing circuitry of the implant device; and
the stimulating processing circuitry is configured to receive sensory feedback data from the prosthetic controller, generate processed sensory signal data based on the sensory feedback data, and transmit electrical signals to the at least one stimulating electrode corresponding to the processed sensory signal data.

12. The method of claim 11, wherein the at least one sensing electrode has a diameter less than 1.5 millimeters, a recording contact length of 1 to 2 centimeters, and a bipolar inter-contact spacing less than 2 centimeters.

13. The method of claim 11, wherein the at least one sensing electrode has a recording contact length of less than 2 millimeters.

14. The method of claim 11, wherein the at least one sensing electrode is a high-resolution electrode having eight contacts that each have a recording contact length of 0.2 millimeters or less.

15. The method of claim 1, wherein the at least one stimulating electrode is a multi-contact electrode having more than two electrode contacts configured to transmit electrical signals to the dermal graft portion of the composite tissue graft.

16. The method of claim 1, wherein the sensing processing circuitry is included on a first application-specific integrated circuit (ASIC) of the implant device and the stimulating processing circuitry is included on a second ASIC of the implant device.

17. The method of claim 1, further comprising:
receiving, with the sensing processing circuitry, additional electrical signals received from at least one additional sensing electrode attached to an additional composite tissue graft, the additional composite tissue graft being surgically attached to surround an additional nerve of the subject, the additional composite tissue graft including an additional muscle graft portion and an additional dermal graft portion, the at least one additional sensing electrode being attached to the additional muscle graft portion of the additional composite tissue graft, the additional composite tissue graft being surgically attached to the subject such that the additional composite tissue graft is entirely surrounded by and in direct contact with non-grafted tissue of the subject, the additional muscle graft potion and the additional dermal graft portion each being an additional autograft of tissue harvested from the subject, devascularized, and deinnervated prior to being surgically attached to the subject, the additional nerve having reinnervated the additional muscle graft portion and the additional dermal graft portion of the additional composite tissue graft subsequent to the additional composite tissue graft being surgically attached to the additional nerve; and
processing, with the sensing processing circuitry, the additional electrical signals received from the at least one additional sensing electrode;
generating, with the sensing processing circuitry, additional processed motor signal data corresponding to the additional electrical signals received from the at least one additional sensing electrode; and
transmitting, with the sensing processing circuitry, the additional processed motor signal data to the prosthetic controller, the prosthetic controller being configured to control the prosthetic device based on the additional processed motor signal data transmitted from the additional sensing processing circuitry of the implant device.

18. The method of claim 11, further comprising:
transmitting, with the stimulating processing circuitry, additional electrical signals to at least one additional sensing electrode attached to an additional composite tissue graft, the additional composite tissue graft being surgically attached to surround an additional nerve of the subject, the additional composite tissue graft including an additional muscle graft portion and an additional dermal graft portion, the at least one additional sensing electrode being attached to the additional dermal graft portion of the additional composite tissue graft, the additional composite tissue graft being surgically attached to the subject such that the additional composite tissue graft is entirely surrounded by and in direct contact with non-grafted tissue of the subject, the additional muscle graft potion and the additional dermal graft portion each being an additional autograft of tissue harvested from the subject, devascularized, and deinnervated prior to being surgically attached to the subject, the additional nerve having reinnervated the additional muscle graft portion and the additional dermal graft portion of the additional composite tissue graft subsequent to the additional composite tissue graft being surgically attached to the additional nerve; and
receiving, with the stimulating processing circuitry, additional sensory feedback data from the prosthetic controller;
generating, with the stimulating processing circuitry, additional processed sensory signal data based on the additional sensory feedback data; and
transmitting, with the stimulating processing circuitry, the additional electrical signals to the at least one additional stimulating electrode corresponding to the additional processed sensory signal data.

19. The method of claim 11, wherein the sensing processing circuitry is included within a first module and the stimulating processing circuitry is included within a second module, the first and second module being in wireless communication with each other via at least one of a wireless body area network (BAN) or a wired network implanted within the subject.

20. The method of claim 11, wherein the electrical signals received from the muscle portion of the composite tissue graft have a voltage amplitude of greater than or equal to about 150 microvolts.
